SEQUENCE LISTING (277 sequences)

SEQ ID NO: 1 - protein, 325 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 2 - protein, 201 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 3 - protein, 348 aa, Mus musculus
MAPCVAGWLGGFLLVPALGIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPSWIVAIILIVSVLVVFLFLLGCFVVLWLIYKKTKHTFRSGTSLPQHLKEFLGHPHHSTFLLFSFPPPEEAEVFDKLSIISEESEGSKQSPEDNCASEPPSDPGPRELESKDEAPSPPHDDPKLLTSTSEV

SEQ ID NO: 4 - protein, 201 aa, Mus musculus
MIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPS

SEQ ID NO: 5 - protein, 460 aa, Homo sapiens
MAGPERWGPLLLCLLQAAPGLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 6 - protein, 208 aa, Homo sapiens
RPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWA

SEQ ID NO: 7-8 - skipped (2 entries)

SEQ ID NO: 9 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 10-12 - skipped (3 entries)

SEQ ID NO: 13 - protein, 4 aa, Artificial Sequence
  Description of Artificial Sequence: Synthetic peptide
GGGS

SEQ ID NO: 14 - protein, 5 aa, Artificial Sequence
  Description of Artificial Sequence: Synthetic peptide
GGGGS

SEQ ID NO: 15 - protein, 4 aa, Artificial Sequence
  Description of Artificial Sequence: Synthetic peptide
GGSG

SEQ ID NO: 16 - protein, 5 aa, Artificial Sequence
  Description of Artificial Sequence: Synthetic peptide
GGSGG

SEQ ID NO: 17 - protein, 5 aa, Artificial Sequence
  Description of Artificial Sequence: Synthetic peptide
GSGSG

SEQ ID NO: 18 - protein, 5 aa, Artificial Sequence
  Description of Artificial Sequence: Synthetic peptide
GSGGG

SEQ ID NO: 19 - protein, 5 aa, Artificial Sequence
  Description of Artificial Sequence: Synthetic peptide
GGGSG

SEQ ID NO: 20 - protein, 5 aa, Artificial Sequence
  Description of Artificial Sequence: Synthetic peptide
GSSSG

SEQ ID NO: 21 - protein, 8 aa, Artificial Sequence
  Description of Artificial Sequence: Synthetic peptide
GGSGGGSG

SEQ ID NO: 22 - protein, 12 aa, Artificial Sequence
  Description of Artificial Sequence: Synthetic peptide
GGSGGGSGGGSG

SEQ ID NO: 23 - protein, 16 aa, Artificial Sequence
  Description of Artificial Sequence: Synthetic peptide
GGSGGGSGGGSGGGSG

SEQ ID NO: 24 - protein, 20 aa, Artificial Sequence
  Description of Artificial Sequence: Synthetic peptide
GGSGGGSGGGSGGGSGGGSG

SEQ ID NO: 25 - protein, 9 aa, Artificial Sequence
  Description of Artificial Sequence: Synthetic peptide
YTYSSGCMG

SEQ ID NO: 26 - protein, 9 aa, Artificial Sequence
  Description of Artificial Sequence: Synthetic peptide
YTYSSYCMG

SEQ ID NO: 27 - protein, 9 aa, Artificial Sequence
  Description of Artificial Sequence: Synthetic peptide
YTYNSYCMG

SEQ ID NO: 28 - protein, 9 aa, Artificial Sequence
  Description of Artificial Sequence: Synthetic peptide
YLYSIDYMA

SEQ ID NO: 29 - protein, 9 aa, Artificial Sequence
  Description of Artificial Sequence: Synthetic peptide
YTYSSYCMG

SEQ ID NO: 30 - protein, 9 aa, Artificial Sequence
  Description of Artificial Sequence: Synthetic peptide
DLYSTNYVA

SEQ ID NO: 31 - protein, 9 aa, Artificial Sequence
  Description of Artificial Sequence: Synthetic peptide
YTYSSGCMG

SEQ ID NO: 32 - protein, 9 aa, Artificial Sequence
  Description of Artificial Sequence: Synthetic peptide
YSYSSYCMG

SEQ ID NO: 33 - protein, 9 aa, Artificial Sequence
  Description of Artificial Sequence: Synthetic peptide
YTYSSGCMG

SEQ ID NO: 34 - protein, 9 aa, Artificial Sequence
  Description of Artificial Sequence: Synthetic peptide
YTYSSYCMG

SEQ ID NO: 35 - protein, 9 aa, Artificial Sequence
  Description of Artificial Sequence: Synthetic peptide
YTYSSYCMG

SEQ ID NO: 36 - protein, 9 aa, Artificial Sequence
  Description of Artificial Sequence: Synthetic peptide
YTYSSYCMG

SEQ ID NO: 37 - protein, 9 aa, Artificial Sequence
  Description of Artificial Sequence: Synthetic peptide
YTYSSYCMG

SEQ ID NO: 38 - protein, 9 aa, Artificial Sequence
  Description of Artificial Sequence: Synthetic peptide
YTYSSGCMG

SEQ ID NO: 39 - protein, 9 aa, Artificial Sequence
  Description of Artificial Sequence: Synthetic peptide
YTASVNYMG

SEQ ID NO: 40 - protein, 9 aa, Artificial Sequence
  Description of Artificial Sequence: Synthetic peptide
YTHSSYCMG

SEQ ID NO: 41 - protein, 9 aa, Artificial Sequence
  Description of Artificial Sequence: Synthetic peptide
YTASVNYMG

SEQ ID NO: 42 - protein, 9 aa, Artificial Sequence
  Description of Artificial Sequence: Synthetic peptide
DTYSSYCMG

SEQ ID NO: 43 - protein, 9 aa, Artificial Sequence
  Description of Artificial Sequence: Synthetic peptide
YTYSSYCMG

SEQ ID NO: 44 - protein, 9 aa, Artificial Sequence
  Description of Artificial Sequence: Synthetic peptide
YTYSSYCMG

SEQ ID NO: 45 - protein, 9 aa, Artificial Sequence
  Description of Artificial Sequence: Synthetic peptide
YTASNNCMG

SEQ ID NO: 46 - protein, 9 aa, Artificial Sequence
  Description of Artificial Sequence: Synthetic peptide
YTDSRYCMG

SEQ ID NO: 47 - protein, 9 aa, Artificial Sequence
  Description of Artificial Sequence: Synthetic peptide
YTYSSYCMG

SEQ ID NO: 48 - protein, 9 aa, Artificial Sequence
  Description of Artificial Sequence: Synthetic peptide
YTYSSYCMG

SEQ ID NO: 49 - protein, 9 aa, Artificial Sequence
  Description of Artificial Sequence: Synthetic peptide
YTYSSYCMG

SEQ ID NO: 50 - protein, 9 aa, Artificial Sequence
  Description of Artificial Sequence: Synthetic peptide
YSYSSYCMG

SEQ ID NO: 51 - protein, 9 aa, Artificial Sequence
  Description of Artificial Sequence: Synthetic peptide
YTYLRGCMG

SEQ ID NO: 52 - protein, 16 aa, Artificial Sequence
  Description of Artificial Sequence: Synthetic peptide
AINSDGSTSYADSVKG

SEQ ID NO: 53 - protein, 16 aa, Artificial Sequence
  Description of Artificial Sequence: Synthetic peptide
AIDSDGSTRYADSVKG

SEQ ID NO: 54 - protein, 16 aa, Artificial Sequence
  Description of Artificial Sequence: Synthetic peptide
TIDSDGMTRYADSVKG

SEQ ID NO: 55 - protein, 17 aa, Artificial Sequence
  Description of Artificial Sequence: Synthetic peptide
VIYTASGATFYPDSVKG

SEQ ID NO: 56 - protein, 16 aa, Artificial Sequence
  Description of Artificial Sequence: Synthetic peptide
HIDSDGSTTYADSVKG

SEQ ID NO: 57 - protein, 17 aa, Artificial Sequence
  Description of Artificial Sequence: Synthetic peptide
VIYTASGATLYSDSVKG

SEQ ID NO: 58 - protein, 16 aa, Artificial Sequence
  Description of Artificial Sequence: Synthetic peptide
TINSDGSTNYADSVKG

SEQ ID NO: 59 - protein, 16 aa, Artificial Sequence
  Description of Artificial Sequence: Synthetic peptide
AIASDGSTSYADSVKG

SEQ ID NO: 60 - protein, 16 aa, Artificial Sequence
  Description of Artificial Sequence: Synthetic peptide
TINSDGSTNYADSVKG

SEQ ID NO: 61 - protein, 16 aa, Artificial Sequence
  Description of Artificial Sequence: Synthetic peptide
HIDSDGSTTYADSVKG

SEQ ID NO: 62 - protein, 16 aa, Artificial Sequence
  Description of Artificial Sequence: Synthetic peptide
AIDSDGSTRYADSVKG

SEQ ID NO: 63 - protein, 16 aa, Artificial Sequence
  Description of Artificial Sequence: Synthetic peptide
HIDSDGSTSYADSVKG

SEQ ID NO: 64 - protein, 16 aa, Artificial Sequence
  Description of Artificial Sequence: Synthetic peptide
HIDSDGSTSYADSVKG

SEQ ID NO: 65 - protein, 16 aa, Artificial Sequence
  Description of Artificial Sequence: Synthetic peptide
TINSDGSTNYADSVKG

SEQ ID NO: 66 - protein, 17 aa, Artificial Sequence
  Description of Artificial Sequence: Synthetic peptide
TIFTGAGTTYYANSVKG

SEQ ID NO: 67 - protein, 16 aa, Artificial Sequence
  Description of Artificial Sequence: Synthetic peptide
AIDVDGSTTYADSVKG

SEQ ID NO: 68 - protein, 17 aa, Artificial Sequence
  Description of Artificial Sequence: Synthetic peptide
TIFTGAGTTYYANSVKG

SEQ ID NO: 69 - protein, 16 aa, Artificial Sequence
  Description of Artificial Sequence: Synthetic peptide
FIDSDGSTRYADSVEG

SEQ ID NO: 70 - protein, 16 aa, Artificial Sequence
  Description of Artificial Sequence: Synthetic peptide
HIDSDGSTSYADSVKG

SEQ ID NO: 71 - protein, 16 aa, Artificial Sequence
  Description of Artificial Sequence: Synthetic peptide
HIDSDGSTTYADSVKG

SEQ ID NO: 72 - protein, 16 aa, Artificial Sequence
  Description of Artificial Sequence: Synthetic peptide
VIFTGAGTSYYDSSVG

SEQ ID NO: 73 - protein, 16 aa, Artificial Sequence
  Description of Artificial Sequence: Synthetic peptide
HIDSDGSTSYADSVKG

SEQ ID NO: 74 - protein, 16 aa, Artificial Sequence
  Description of Artificial Sequence: Synthetic peptide
AIDSDGSTRYADSVKG

SEQ ID NO: 75 - protein, 16 aa, Artificial Sequence
  Description of Artificial Sequence: Synthetic peptide
HIDSDGSTTYADSVKG

SEQ ID NO: 76 - protein, 16 aa, Artificial Sequence
  Description of Artificial Sequence: Synthetic peptide
HIDSDGSTTYADSVKG

SEQ ID NO: 77 - protein, 16 aa, Artificial Sequence
  Description of Artificial Sequence: Synthetic peptide
TIDSDGMTRYADSVKG

SEQ ID NO: 78 - protein, 17 aa, Artificial Sequence
  Description of Artificial Sequence: Synthetic peptide
VMDVVGDRRSYIDSVKG

SEQ ID NO: 79 - protein, 18 aa, Artificial Sequence
  Description of Artificial Sequence: Synthetic peptide
EPYCSGGYPRWSVAEFGY

SEQ ID NO: 80 - protein, 18 aa, Artificial Sequence
  Description of Artificial Sequence: Synthetic peptide
EPYCSGGYKRTMVAEFGY

SEQ ID NO: 81 - protein, 13 aa, Artificial Sequence
  Description of Artificial Sequence: Synthetic peptide
DADCTIAAMTTNP

SEQ ID NO: 82 - protein, 16 aa, Artificial Sequence
  Description of Artificial Sequence: Synthetic peptide
VRKTDSYLFDAQSFTY

SEQ ID NO: 83 - protein, 16 aa, Artificial Sequence
  Description of Artificial Sequence: Synthetic peptide
DPIPGPGYCDGGPNKY

SEQ ID NO: 84 - protein, 16 aa, Artificial Sequence
  Description of Artificial Sequence: Synthetic peptide
VRKTGHYLFDAQSFTY

SEQ ID NO: 85 - protein, 18 aa, Artificial Sequence
  Description of Artificial Sequence: Synthetic peptide
EPYCSGGYPRWSVAEFGY

SEQ ID NO: 86 - protein, 18 aa, Artificial Sequence
  Description of Artificial Sequence: Synthetic peptide
EPWCTGGYSRLTPAEYGY

SEQ ID NO: 87 - protein, 18 aa, Artificial Sequence
  Description of Artificial Sequence: Synthetic peptide
EPYCSGGYPRWSVAEFGY

SEQ ID NO: 88 - protein, 16 aa, Artificial Sequence
  Description of Artificial Sequence: Synthetic peptide
DPIPGPGYCDGGPNKY

SEQ ID NO: 89 - protein, 18 aa, Artificial Sequence
  Description of Artificial Sequence: Synthetic peptide
EPYCSGGYKRTMVAEFGY

SEQ ID NO: 90 - protein, 16 aa, Artificial Sequence
  Description of Artificial Sequence: Synthetic peptide
DPIPGPGYCDGGPNKY

SEQ ID NO: 91 - protein, 16 aa, Artificial Sequence
  Description of Artificial Sequence: Synthetic peptide
DPIPGPGYCDGGPNNY

SEQ ID NO: 92 - protein, 18 aa, Artificial Sequence
  Description of Artificial Sequence: Synthetic peptide
EPYCSGGYPRWSVAEFGY

SEQ ID NO: 93 - protein, 16 aa, Artificial Sequence
  Description of Artificial Sequence: Synthetic peptide
DFRGGLLYRPAYEYTY

SEQ ID NO: 94 - protein, 18 aa, Artificial Sequence
  Description of Artificial Sequence: Synthetic peptide
EFADCSSNYFLPPGAVRY

SEQ ID NO: 95 - protein, 16 aa, Artificial Sequence
  Description of Artificial Sequence: Synthetic peptide
DFRGGLLYRPAYEYTY

SEQ ID NO: 96 - protein, 18 aa, Artificial Sequence
  Description of Artificial Sequence: Synthetic peptide
EPYCSGGYHRKEMAEFGY

SEQ ID NO: 97 - protein, 16 aa, Artificial Sequence
  Description of Artificial Sequence: Synthetic peptide
DPIPGPGYCDGGPNKY

SEQ ID NO: 98 - protein, 16 aa, Artificial Sequence
  Description of Artificial Sequence: Synthetic peptide
DPIPGPGYCDGGPNKY

SEQ ID NO: 99 - protein, 16 aa, Artificial Sequence
  Description of Artificial Sequence: Synthetic peptide
EDDCTLLLMTPNPDDQ

SEQ ID NO: 100 - protein, 16 aa, Artificial Sequence
  Description of Artificial Sequence: Synthetic peptide
DPIPGPGYCDGGPNKY

SEQ ID NO: 101 - protein, 18 aa, Artificial Sequence
  Description of Artificial Sequence: Synthetic peptide
EPYCSGGYKRTMVAEFGF

SEQ ID NO: 102 - protein, 16 aa, Artificial Sequence
  Description of Artificial Sequence: Synthetic peptide
DPIPGPGYCDGGPNNY

SEQ ID NO: 103 - protein, 16 aa, Artificial Sequence
  Description of Artificial Sequence: Synthetic peptide
DPIPGPGYCDGGPNNY

SEQ ID NO: 104 - protein, 16 aa, Artificial Sequence
  Description of Artificial Sequence: Synthetic peptide
PLYDCDSGAVGRNPPY

SEQ ID NO: 105 - protein, 13 aa, Artificial Sequence
  Description of Artificial Sequence: Synthetic peptide
GPNCVGWRSGLDY

SEQ ID NO: 106 - protein, 9 aa, Artificial Sequence
  Description of Artificial Sequence: Synthetic peptide
YTASSICMG

SEQ ID NO: 107 - protein, 9 aa, Artificial Sequence
  Description of Artificial Sequence: Synthetic peptide
DTYSRKYIA

SEQ ID NO: 108 - protein, 9 aa, Artificial Sequence
  Description of Artificial Sequence: Synthetic peptide
YASCSRAMR

SEQ ID NO: 109 - protein, 9 aa, Artificial Sequence
  Description of Artificial Sequence: Synthetic peptide
YTYNRRFMG

SEQ ID NO: 110 - protein, 9 aa, Artificial Sequence
  Description of Artificial Sequence: Synthetic peptide
YIALNACMA

SEQ ID NO: 111 - protein, 9 aa, Artificial Sequence
  Description of Artificial Sequence: Synthetic peptide
YTYNGKCMA

SEQ ID NO: 112 - protein, 17 aa, Artificial Sequence
  Description of Artificial Sequence: Synthetic peptide
VITTAASGTYYADSVNG

SEQ ID NO: 113 - protein, 17 aa, Artificial Sequence
  Description of Artificial Sequence: Synthetic peptide
VMYTPGSATYYTDTVMG

SEQ ID NO: 114 - protein, 16 aa, Artificial Sequence
  Description of Artificial Sequence: Synthetic peptide
YIDGVGSTGYADSVKG

SEQ ID NO: 115 - protein, 17 aa, Artificial Sequence
  Description of Artificial Sequence: Synthetic peptide
IIYTPNSSTFYADSVTG

SEQ ID NO: 116 - protein, 17 aa, Artificial Sequence
  Description of Artificial Sequence: Synthetic peptide
TIVTDGSRTYYADSVKG

SEQ ID NO: 117 - protein, 17 aa, Artificial Sequence
  Description of Artificial Sequence: Synthetic peptide
GIYTGGSSTYYADSVKG

SEQ ID NO: 118 - protein, 18 aa, Artificial Sequence
  Description of Artificial Sequence: Synthetic peptide
TRRGGDCLDPLQTPAYNT

SEQ ID NO: 119 - protein, 14 aa, Artificial Sequence
  Description of Artificial Sequence: Synthetic peptide
KASGSMFNFRDYTY

SEQ ID NO: 120 - protein, 13 aa, Artificial Sequence
  Description of Artificial Sequence: Synthetic peptide
GCRADGSNSLDNY

SEQ ID NO: 121 - protein, 16 aa, Artificial Sequence
  Description of Artificial Sequence: Synthetic peptide
ARIASMTELSVRDMDY

SEQ ID NO: 122 - protein, 17 aa, Artificial Sequence
  Description of Artificial Sequence: Synthetic peptide
DRRCPVSRAPYEYELRY

SEQ ID NO: 123 - protein, 14 aa, Artificial Sequence
  Description of Artificial Sequence: Synthetic peptide
SRSCSDLRRRSIAY

SEQ ID NO: 124 - protein, 9 aa, Artificial Sequence
  Description of Artificial Sequence: Synthetic peptide
YISSSYCMA

SEQ ID NO: 125 - protein, 9 aa, Artificial Sequence
  Description of Artificial Sequence: Synthetic peptide
FTFSNYGMS

SEQ ID NO: 126 - protein, 9 aa, Artificial Sequence
  Description of Artificial Sequence: Synthetic peptide
FTFSDYAMS

SEQ ID NO: 127 - protein, 9 aa, Artificial Sequence
  Description of Artificial Sequence: Synthetic peptide
STDNIKYMG

SEQ ID NO: 128 - protein, 9 aa, Artificial Sequence
  Description of Artificial Sequence: Synthetic peptide
FTFSNATMS

SEQ ID NO: 129 - protein, 9 aa, Artificial Sequence
  Description of Artificial Sequence: Synthetic peptide
FTFSDYAMS

SEQ ID NO: 130 - protein, 9 aa, Artificial Sequence
  Description of Artificial Sequence: Synthetic peptide
FTFSNYGMS

SEQ ID NO: 131 - protein, 10 aa, Artificial Sequence
  Description of Artificial Sequence: Synthetic peptide
YTISRSDCMG

SEQ ID NO: 132 - protein, 9 aa, Artificial Sequence
  Description of Artificial Sequence: Synthetic peptide
FTFSNYGMS

SEQ ID NO: 133 - protein, 11 aa, Artificial Sequence
  Description of Artificial Sequence: Synthetic peptide
YISSRSTYCMG

SEQ ID NO: 134 - protein, 9 aa, Artificial Sequence
  Description of Artificial Sequence: Synthetic peptide
FTYSSYCMG

SEQ ID NO: 135 - protein, 11 aa, Artificial Sequence
  Description of Artificial Sequence: Synthetic peptide
YISSRSTYCMG

SEQ ID NO: 136 - protein, 9 aa, Artificial Sequence
  Description of Artificial Sequence: Synthetic peptide
FTFSNATMS

SEQ ID NO: 137 - protein, 10 aa, Artificial Sequence
  Description of Artificial Sequence: Synthetic peptide
YISRSSYCMG

SEQ ID NO: 138 - protein, 16 aa, Artificial Sequence
  Description of Artificial Sequence: Synthetic peptide
GVTRDGKTYYGDSVKG

SEQ ID NO: 139 - protein, 17 aa, Artificial Sequence
  Description of Artificial Sequence: Synthetic peptide
GINSGGDDTFYTDSVKG

SEQ ID NO: 140 - protein, 16 aa, Artificial Sequence
  Description of Artificial Sequence: Synthetic peptide
AIGRDGSTFYPDSVKG

SEQ ID NO: 141 - protein, 17 aa, Artificial Sequence
  Description of Artificial Sequence: Synthetic peptide
AVYTSGGAVVYADSVKG

SEQ ID NO: 142 - protein, 17 aa, Artificial Sequence
  Description of Artificial Sequence: Synthetic peptide
AISNSRGTKYYAAFVKG

SEQ ID NO: 143 - protein, 16 aa, Artificial Sequence
  Description of Artificial Sequence: Synthetic peptide
AIGRDGSTFYPDSVKG

SEQ ID NO: 144 - protein, 17 aa, Artificial Sequence
  Description of Artificial Sequence: Synthetic peptide
GINSGGDDTFYTDSVKG

SEQ ID NO: 145 - protein, 16 aa, Artificial Sequence
  Description of Artificial Sequence: Synthetic peptide
RIGSDGTTSYADSVKE

SEQ ID NO: 146 - protein, 17 aa, Artificial Sequence
  Description of Artificial Sequence: Synthetic peptide
GINSGGDDTFYTDSVKG

SEQ ID NO: 147 - protein, 16 aa, Artificial Sequence
  Description of Artificial Sequence: Synthetic peptide
VVTGDSRTYYGDSVKG

SEQ ID NO: 148 - protein, 16 aa, Artificial Sequence
  Description of Artificial Sequence: Synthetic peptide
AIDSDGSTSYADSVKG

SEQ ID NO: 149 - protein, 16 aa, Artificial Sequence
  Description of Artificial Sequence: Synthetic peptide
IVTGDSRTYYGDSVRG

SEQ ID NO: 150 - protein, 17 aa, Artificial Sequence
  Description of Artificial Sequence: Synthetic peptide
AISNSRGTKYYAAFVKG

SEQ ID NO: 151 - protein, 16 aa, Artificial Sequence
  Description of Artificial Sequence: Synthetic peptide
IVTGDGRTYYGDSVKG

SEQ ID NO: 152 - protein, 19 aa, Artificial Sequence
  Description of Artificial Sequence: Synthetic peptide
GPPPCITSMPAGGDYGYRY

SEQ ID NO: 153 - protein, 7 aa, Artificial Sequence
  Description of Artificial Sequence: Synthetic peptide
GASGMIP

SEQ ID NO: 154 - protein, 7 aa, Artificial Sequence
  Description of Artificial Sequence: Synthetic peptide
EEPGSSS

SEQ ID NO: 155 - protein, 18 aa, Artificial Sequence
  Description of Artificial Sequence: Synthetic peptide
SRAPAPPRLLLQRALVEY

SEQ ID NO: 156 - protein, 12 aa, Artificial Sequence
  Description of Artificial Sequence: Synthetic peptide
DWKTSYSDYDLS

SEQ ID NO: 157 - protein, 7 aa, Artificial Sequence
  Description of Artificial Sequence: Synthetic peptide
EEPGSSS

SEQ ID NO: 158 - protein, 7 aa, Artificial Sequence
  Description of Artificial Sequence: Synthetic peptide
GASGMIP

SEQ ID NO: 159 - protein, 20 aa, Artificial Sequence
  Description of Artificial Sequence: Synthetic peptide
TALLLGRGSACHKEVSVFSW

SEQ ID NO: 160 - protein, 7 aa, Artificial Sequence
  Description of Artificial Sequence: Synthetic peptide
GASGMIP

SEQ ID NO: 161 - protein, 19 aa, Artificial Sequence
  Description of Artificial Sequence: Synthetic peptide
GPPPCITTMPAGGDYGYRY

SEQ ID NO: 162 - protein, 16 aa, Artificial Sequence
  Description of Artificial Sequence: Synthetic peptide
DGEYNDYVCWSTGLRY

SEQ ID NO: 163 - protein, 19 aa, Artificial Sequence
  Description of Artificial Sequence: Synthetic peptide
GPPPCITSMPAGGDYGYRY

SEQ ID NO: 164 - protein, 12 aa, Artificial Sequence
  Description of Artificial Sequence: Synthetic peptide
DWKTSYSDYDLS

SEQ ID NO: 165 - protein, 19 aa, Artificial Sequence
  Description of Artificial Sequence: Synthetic peptide
GPPPCITTMPAGGDYGYRY

SEQ ID NO: 166 - protein, 126 aa, Artificial Sequence
  Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSS

SEQ ID NO: 167 - protein, 126 aa, Artificial Sequence
  Description of Artificial Sequence: Synthetic polypeptide
QVQLQESGGGSVQAGGSLRLSCAASGYTYSSYCMGWFRQAPGKEREGVAAIDSDGSTRYADSVKGRFTISKDNAKNTLYLQMNSLKPEDTAMYYCAAEPYCSGGYKRTMVAEFGYWGQGTQVTVSS

SEQ ID NO: 168 - protein, 121 aa, Artificial Sequence
  Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSS

SEQ ID NO: 169 - protein, 125 aa, Artificial Sequence
  Description of Artificial Sequence: Synthetic polypeptide
QVQLQESGGGSIQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSS

SEQ ID NO: 170 - protein, 124 aa, Artificial Sequence
  Description of Artificial Sequence: Synthetic polypeptide
QVQLQESGGGLVQPGGSLRLSCAASGYTYSSYCMGWFRQAPGKEREGVAHIDSDGSTTYADSVKGRFTISKDNAKNTLYLQMNSLKPEDTAMYYCAADPIPGPGYCDGGPNKYWGQGTQVTVSS

SEQ ID NO: 171 - protein, 125 aa, Artificial Sequence
  Description of Artificial Sequence: Synthetic polypeptide
QVQLQESGGGSIQAGGSLTLSCAASRDLYSTNYVAWFRQSPGKEREAVAVIYTASGATLYSDSVKGRFTISQDNAKMTVYLQMNSLKSEDTAMYYCAAVRKTGHYLFDAQSFTYWGQGTQVTVSS

SEQ ID NO: 172 - protein, 126 aa, Artificial Sequence
  Description of Artificial Sequence: Synthetic polypeptide
QVQLQESGGGSVQAGGSLRLSCAASGYTYSSGCMGWFRQAPGKEREGVATINSDGSTNYADSVKGRFTISKDNAKNTLYLQMNSLKPEDTAMYYCAAEPYCSGGYPRWSVAEFGYWGQGTQVTVSS

SEQ ID NO: 173 - protein, 126 aa, Artificial Sequence
  Description of Artificial Sequence: Synthetic polypeptide
QVQLQESGGGSVQAGGSLRLSCAASGYSYSSYCMGWFRQAPGKEREGVAAIASDGSTSYADSVKGRFAISKDNAKNTLYLQMASLKPEDTAMYYCAAEPWCTGGYSRLTPAEYGYWGQGTQVTVSS

SEQ ID NO: 174 - protein, 126 aa, Artificial Sequence
  Description of Artificial Sequence: Synthetic polypeptide
QVQLQESGGGLVQPGGSLRLSCAASGYTYSSGCMGWFRQAPGKEREGVATINSDGSTNYADSVKGRFTISKDNAKNTLYLQMNSLKPEDTAMYYCAAEPYCSGGYPRWSVAEFGYWGQGTQVTVSS

SEQ ID NO: 175 - protein, 124 aa, Artificial Sequence
  Description of Artificial Sequence: Synthetic polypeptide
QVQLQESGGGLVQPGGSLRLSCAASGYTYSSYCMGWFRQAPGKEREGVAHIDSDGSTTYADSVKGRFAISKDNAKNTLYLQMNSLKPEDTAMYYCAADPIPGPGYCDGGPNKYWGQGTQVTVSS

SEQ ID NO: 176 - protein, 126 aa, Artificial Sequence
  Description of Artificial Sequence: Synthetic polypeptide
QVQLQESGGGSVQAGGSLRLSCAASGYTYSSYCMGWFRQAPGKGREGVAAIDSDGSTRYADSVKGRFTISKDNAKNTLYLQMNSLKPEDTAMYYCAAEPYCSGGYKRTMVAEFGYWGQGTQVTVSS

SEQ ID NO: 177 - protein, 124 aa, Artificial Sequence
  Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSS

SEQ ID NO: 178 - protein, 124 aa, Artificial Sequence
  Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNNYWGQGTQVTVSS

SEQ ID NO: 179 - protein, 126 aa, Artificial Sequence
  Description of Artificial Sequence: Synthetic polypeptide
QVQLQESGGGSVQAGGSLRLSCAASGYTYSSGCMGWFRQAPGKEREGVATINSDGSTNYADSVKGRFTISKDNAKNTLYLQMNSLKPEDTGMYYCAAEPYCSGGYPRWSVAEFGYWGQGTQVTVSS

SEQ ID NO: 180 - protein, 125 aa, Artificial Sequence
  Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSS

SEQ ID NO: 181 - protein, 126 aa, Artificial Sequence
  Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSS

SEQ ID NO: 182 - protein, 125 aa, Artificial Sequence
  Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAMYYCAVDFRGGLLYRPAYEYTYRGQGTQVTVSS

SEQ ID NO: 183 - protein, 126 aa, Artificial Sequence
  Description of Artificial Sequence: Synthetic polypeptide
QVQLQESGGGSVQAGGSLRLSCAASGDTYSSYCMGWFRQAPGKEREGVAFIDSDGSTRYADSVEGRFTISKDNAKNTLYLQMNSLKPEDTAMYYCAAEPYCSGGYHRKEMAEFGYWGQGTQVTVSS

SEQ ID NO: 184 - protein, 124 aa, Artificial Sequence
  Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYRGQGTQVTVSS

SEQ ID NO: 185 - protein, 124 aa, Artificial Sequence
  Description of Artificial Sequence: Synthetic polypeptide
QVQLQESGGGSVQAGGSLRLSCAASGYTYSSYCMGWFRQAPGKEREGVAHIDSDGSTTYADSVKGRFAISKDNAKNTLYLQMNSLKPEDTAMYYCAADPIPGPGYCDGGPNKYWGQGTQVTVSS

SEQ ID NO: 186 - protein, 121 aa, Artificial Sequence
  Description of Artificial Sequence: Synthetic polypeptide
QVQLQESGGGSVQAGGSLRLSCTGSGYTASNNCMGWFRQAPGKEREGVAVIFTGAGTSYYDSSVGRLFISSQDAASTLDQLLMSLLPDDTAVMYCGAEDDCTLLLMTPNPDDQWSRLSVSS

SEQ ID NO: 187 - protein, 124 aa, Artificial Sequence
  Description of Artificial Sequence: Synthetic polypeptide
QVQLQESGGGSVQAGGSLRLSCAASGYTDSRYCMGWFRKAPGKEREGVAHIDSDGSTSYADSVKGRFTISKDNAKNTLYLQMNSLKPEDTAMYYCAADPIPGPGYCDGGPNKYWGQGTQVTVSS

SEQ ID NO: 188 - protein, 126 aa, Artificial Sequence
  Description of Artificial Sequence: Synthetic polypeptide
QVQLQESGGGSVQAGGSLRLSCAASGYTYSSYCMGWFRQAPGKEREGVAAIDSDGSTRYADSVKGRFTISKDNAKKILYLQMNSLKVEDTAMYYCAAEPYCSGGYKRTMVAEFGFWGQGTQVTVSS

SEQ ID NO: 189 - protein, 124 aa, Artificial Sequence
  Description of Artificial Sequence: Synthetic polypeptide
QVQLQESGGGSVQAGGSLKLSCAASGYTYSSYCMGWFRQAPGKEREGVAHIDSDGSTTYADSVKGRFTISKDNAKNTLYLQMNSLKPEDTAMYYCAADPIPGPGYCDGGPNNYWGQGTQVTVSS

SEQ ID NO: 190 - protein, 124 aa, Artificial Sequence
  Description of Artificial Sequence: Synthetic polypeptide
QVQLQESGGGSVQAGGSLRLSCAASGYTYSSYCMGWFRQAPGKEREGIAHIDSDGSTTYADSVKGRFTISKDNAKNTLYLQMNSLKPEDTAMYYCAADPIPGPGYCDGGPNNYWGQGTQVTVSS

SEQ ID NO: 191 - protein, 124 aa, Artificial Sequence
  Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSS

SEQ ID NO: 192 - protein, 122 aa, Artificial Sequence
  Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSS

SEQ ID NO: 193 - protein, 127 aa, Artificial Sequence
  Description of Artificial Sequence: Synthetic polypeptide
QVQLQESGGGSVQAGGALRLSCTASGYTASSICMGWFRQAPGKERERVAVITTAASGTYYADSVNGRFSISQNNAKNTVYLQMNSLKPDDTAMYYCAATRRGGDCLDPLQTPAYNTWGQGTQVTVSS

SEQ ID NO: 194 - protein, 123 aa, Artificial Sequence
  Description of Artificial Sequence: Synthetic polypeptide
QVQLQESGGGSVQAGGSLRLSCVASGDTYSRKYIAWVRQVPGKEREGVAVMYTPGSATYYTDTVMGRFTISQDNAKNTVYLQMNSLKPEDTAMYFCAAKASGSMFNFRDYTYWGQGTQVTVSS

SEQ ID NO: 195 - protein, 121 aa, Artificial Sequence
  Description of Artificial Sequence: Synthetic polypeptide
QVQLQESGGGSVQAGGSLRLSCATSGYASCSRAMRWYRQAPGKEREFVAYIDGVGSTGYADSVKGRFTISQDNAKYTAYLQMNSLKPEDTAMYYCNRGCRADGSNSLDNYWGQGTQVTVSS

SEQ ID NO: 196 - protein, 125 aa, Artificial Sequence
  Description of Artificial Sequence: Synthetic polypeptide
QVQLQESGGGSVQAGGSLRLSCAASGYTYNRRFMGWFRQAPGKEREGLAIIYTPNSSTFYADSVTGRFTISQDSARNTVYLQMNSLKPEDTAMYYCAAARIASMTELSVRDMDYWGKGTQVTVSS

SEQ ID NO: 197 - protein, 126 aa, Artificial Sequence
  Description of Artificial Sequence: Synthetic polypeptide
QVQLQESGGGSVQAGGSLRLSCTASRYIALNACMAWIRQAPGSEREVVATIVTDGSRTYYADSVKGRFTISQDNAKNTMYLQMNGLKPEDTAMYYCAADRRCPVSRAPYEYELRYWGQGTQVTVSS

SEQ ID NO: 198 - protein, 123 aa, Artificial Sequence
  Description of Artificial Sequence: Synthetic polypeptide
QVQLQESGGGSVQAGGSLRLSCAASGYTYNGKCMAWFRQAPGKEREVVAGIYTGGSSTYYADSVKGRFTISQDNAKNTVYLQMDSLKPEDTAMYYCATSRSCSDLRRRSIAYWGQGTQVTVSS

SEQ ID NO: 199 - protein, 127 aa, Artificial Sequence
  Description of Artificial Sequence: Synthetic polypeptide
QVQLQESGGGSVQAGGSLRLSCASSGYISSSYCMAWFRQAPGKEREGAAGVTRDGKTYYGDSVKGRFAISRDNAKNTLYLQMNSLKPEDTAMYYCAAGPPPCITSMPAGGDYGYRYWGQGTQVTVSS

SEQ ID NO: 200 - protein, 116 aa, Artificial Sequence
  Description of Artificial Sequence: Synthetic polypeptide
QVQLQESGGGLVQPGGSLRLSCAASGFTFSNYGMSWVRQAPGKGLEWVSGINSGGDDTFYTDSVKGRFTISRDNAKNTLYLQMNSLKTEDTAMYYCAMGASGMIPRGQGTQITVSS

SEQ ID NO: 201 - protein, 115 aa, Artificial Sequence
  Description of Artificial Sequence: Synthetic polypeptide
QVQLQESGGGLVQPGGSLRLSCVASGFTFSDYAMSWVRQAPGMGLERVSAIGRDGSTFYPDSVKGRFTISRDNAKNTLYLQLNSLKTEDTAMYYCAKEEPGSSSRGQGTQVTVSS

SEQ ID NO: 202 - protein, 127 aa, Artificial Sequence
  Description of Artificial Sequence: Synthetic polypeptide
QVQLQESGGGSVQLGGSLRLSCLVSGSTDNIKYMGWFRQAPGKEREGVAAVYTSGGAVVYADSVKGRFTISQDDAKNTMYLQMNSLKPEDTAMYYCAASRAPAPPRLLLQRALVEYWGQGTQVTVSS

SEQ ID NO: 203 - protein, 121 aa, Artificial Sequence
  Description of Artificial Sequence: Synthetic polypeptide
QVQLQESGGGLVQPGGSLRLSCAASGFTFSNATMSWVRQAPGKEIEWVSAISNSRGTKYYAAFVKGRFTISRDNAKNTLYLQLNNLKTEDTAMYYCTKDWKTSYSDYDLSDGQGTQVTVSS

SEQ ID NO: 204 - protein, 115 aa, Artificial Sequence
  Description of Artificial Sequence: Synthetic polypeptide
QVQLQESGGGLVQPGGSLRLSCAASGFTFSDYAMSWVRQAPGMGLERVSAIGRDGSTFYPDSVKGRFTISRDNAKNTLYLQLNSLKTEDTAMYYCAKEEPGSSSRGQGTQVTVSS

SEQ ID NO: 205 - protein, 116 aa, Artificial Sequence
  Description of Artificial Sequence: Synthetic polypeptide
QVQLQESGGGLVQPGGSLRLSCAASGFTFSNYGMSWVRQAPGKGLEWVSGINSGGDDTFYTDSVKGRFTISRDNAKNTLYLQMNSLKTEDTAMYYCAMGASGMIPRGQGTQVTVSS

SEQ ID NO: 206 - protein, 129 aa, Artificial Sequence
  Description of Artificial Sequence: Synthetic polypeptide
QVQLQESGGGSVQAGGSLRLSCAVSRYTISRSDCMGWFRQAPGKEREGVARIGSDGTTSYADSVKERFTISKDNAKNILYLQMNSLKPEDTARYYCAATALLLGRGSACHKEVSVFSWWGQGTQVTVSS

SEQ ID NO: 207 - protein, 116 aa, Artificial Sequence
  Description of Artificial Sequence: Synthetic polypeptide
QVQLQESGGGLVQPGGSLRLSCAASGFTFSNYGMSWVRQAPGKGLEWVSGINSGGDDTFYTDSVKGRFTISRDNVKNTLYLQMNSLKTEDTAMYYCAMGASGMIPRGQGTQVTVSS

SEQ ID NO: 208 - protein, 129 aa, Artificial Sequence
  Description of Artificial Sequence: Synthetic polypeptide
QVQLQESGGGSVQAGGSLRLSCASSGYISSRSTYCMGWFRQAPGKEREVAAVVTGDSRTYYGDSVKGRFAISRDNAKNTLYLQMNSLKPEDTAMYYCAAGPPPCITTMPAGGDYGYRYWGQGTQVTVSS

SEQ ID NO: 209 - protein, 124 aa, Artificial Sequence
  Description of Artificial Sequence: Synthetic polypeptide
QVQLQESGGGSVQSGGSLRLSCAASGFTYSSYCMGWFRQAPGKEREGVAAIDSDGSTSYADSVKGRFTISKDNAKNTLYLQMNSLRPEDTAMYYCAADGEYNDYVCWSTGLRYRGQGTQVTVSS

SEQ ID NO: 210 - protein, 129 aa, Artificial Sequence
  Description of Artificial Sequence: Synthetic polypeptide
QVQLQESGGGSVQAGGSLRLSCASSGYISSRSTYCMGWFRQAPGKEREVAAIVTGDSRTYYGDSVRGRFAISRDNAKNTLYLQMNSLKPEDTAMYYCAAGPPPCITSMPAGGDYGYRYWGQGTQVTVSS

SEQ ID NO: 211 - protein, 121 aa, Artificial Sequence
  Description of Artificial Sequence: Synthetic polypeptide
QVQLQESGGGLVQPGSSLRLSCAASGFTFSNATMSWVRQAPGKEIEWVSAISNSRGTKYYAAFVKGRFTISRDNAKNTLYLQLNNLKTEDTAMYYCTKDWKTSYSDYDLSDGQGTQVTVSS

SEQ ID NO: 212 - protein, 128 aa, Artificial Sequence
  Description of Artificial Sequence: Synthetic polypeptide
QVQLQESGGGSVQAGGSLRLSCASSGYISRSSYCMGWFRQAPGKEREVAAIVTGDGRTYYGDSVKGRFAISRDNAKNTLYLQMNSLKPEDTAMYYCVAGPPPCITTMPAGGDYGYRYWGRGTQVTVSS

SEQ ID NO: 213 - DNA, 378 bp, Artificial Sequence
  Description of Artificial Sequence: Synthetic polynucleotide
caggtgcagcttcaggaatcaggcggaggcagcgtgcaggcagggggtagcctgcgtctgtcttgcgcagccagcgggtacacctacagctctggctgtatgggctggtttcgccaagccccaggaaaagaacgggaagccgtggcggctatcaatagcgacggctccacctcctatgctgactccgtcaaaggacgcttcaccattagtaaagataacgccaagaacaccttgtaccttcagatgaactccttgaaaccggaggacaccgcaatgtattactgtgcggctgagccctactgctcaggaggctacccacggtggtcagtggccgagtttggttattggggccagggcacccaagtgactgtgtcctcc

SEQ ID NO: 214 - DNA, 378 bp, Artificial Sequence
  Description of Artificial Sequence: Synthetic polynucleotide
caggtgcaactccaggagtcagggggaggttccgtgcaggctggcggttctctcaggttgtcttgcgcggccagcggctatacgtacagtagctactgcatgggctggttccggcaagcccccggcaaggagcgcgaaggcgtggctgccattgattccgatggatctactaggtatgctgatagtgtaaagggccgcttcacaatctccaaggacaatgccaagaacacactgtatttgcaaatgaactccctcaagcccgaggataccgctatgtactattgcgctgccgaaccatactgttccggtggctataagcgcactatggtggccgagttcggatactggggtcaaggcacacaggtcacagtgtcctct

SEQ ID NO: 215 - DNA, 363 bp, Artificial Sequence
  Description of Artificial Sequence: Synthetic polynucleotide
caggtgcagttgcaggagtccgggggcggtagcgttcaggctggagggtccctgcgtctgagttgtgcggcatctcggtatacttataacagttactgtatgggttggttccgccaggcacctggaaaggagcgggagggggtggcgactattgatagcgacggaatgaccagatatgccgactctgtgaagggaagatttactatctcaaaagataatgccaagaacacactctatttgcagatgaacagcctcaagccagaggataccgctatgtattactgtgctgccgacgctgattgcaccatcgctgccatgacgaccaaccccttgggccagggaacccaagtaaccgtctctagc

SEQ ID NO: 216 - DNA, 375 bp, Artificial Sequence
  Description of Artificial Sequence: Synthetic polynucleotide
caggtccagctccaggaatctggtggcgggtctatccaggcgggtggcagcctgcggctgagttgcgccgcttcccgctacctgtatagtattgattatatggcctggttcaggcagtcaccgggcaaagagcgcgaacccgtcgctgtgatttacacagcctctggtgccaccttctatcccgatagtgtgaagggccggttcactatctctcaagacaacgcgaagatgactgtctatcttcagatgaactctctgaagtccgaggacactgccatgtattactgtgccgctgtgcgcaagacggactcttatctgttcgatgcccagagtttcacttactggggtcagggtactcaggtgaccgtatcctcc

SEQ ID NO: 217 - DNA, 372 bp, Artificial Sequence
  Description of Artificial Sequence: Synthetic polynucleotide
caggtgcagctccaggagtctggtggcgggctggttcagcctgggggttcactccggttgtcctgcgctgcgtctggttatacctactccagctactgtatgggttggttccgccaggcaccggggaaggagagggagggcgtggctcacattgattctgatggctctacgacctacgctgatagcgttaaggggcgcttcactatctccaaggataacgccaagaacaccctgtatctgcaaatgaacagcctgaagccagaagacactgccatgtactattgcgctgccgatcctattcccggtcctggctattgtgacggcggtcctaacaagtactggggccaaggcacacaggtgactgtcagttcc

SEQ ID NO: 218 - DNA, 375 bp, Artificial Sequence
  Description of Artificial Sequence: Synthetic polynucleotide
caggttcaactccaggaatccggcggtggaagcattcaggcgggcggttctttgactctgagctgtgcggcatctcgggacctttacagcactaactatgttgcctggttccggcagtcccccggcaaggaacgcgaagctgtggccgtgatttatacagccagcggcgcaaccctgtatagcgattcagtcaaaggccggttcaccatctcccaggacaacgcgaagatgaccgtgtacctgcaaatgaacagcctgaagtctgaggacactgccatgtattactgcgcagctgtgagaaagaccggacattacctcttcgacgcccaatctttcacctactggggccagggaacccaggtcaccgtctcctct

SEQ ID NO: 219 - DNA, 378 bp, Artificial Sequence
  Description of Artificial Sequence: Synthetic polynucleotide
caggtgcaactccaggagtcaggcggtgggtccgtccaggccggtggctccctgaggctgagttgcgccgcttccggctatacttactccagcggttgcatggggtggttccgccaagcccccggtaaagaacgcgagggagtggctacaattaactccgatggaagcactaactacgccgactctgtgaagggacgcttcaccattagcaaagacaatgctaagaacaccctttaccttcaaatgaacagcctgaagcctgaggataccgctatgtattactgtgccgcagaaccgtattgtagcggtggctaccctcgctggtccgtcgccgagttcggttattggggccaggggacccaagtgactgtttctagc

SEQ ID NO: 220 - DNA, 378 bp, Artificial Sequence
  Description of Artificial Sequence: Synthetic polynucleotide
caggtgcaacttcaggagagcggcgggggctctgtgcaagctggtggctccctgcggctcagctgtgctgcctctgggtattcttacagtagctactgtatgggctggttcagacaggcaccaggcaaggagcgcgagggtgtggcggccatcgcttccgacgggagtaccagctacgccgacagcgttaaaggtaggtttgccatctctaaggataatgcgaagaatacactctaccttcagatggctagtctgaagccagaggataccgccatgtattactgcgcggcagagccctggtgcacgggagggtattcacgcctgaccccggctgagtatggatactgggggcagggcacccaggtgaccgttagctcc

SEQ ID NO: 221 - DNA, 378 bp, Artificial Sequence
  Description of Artificial Sequence: Synthetic polynucleotide
caggtccagttgcaggaaagcggagggggcctggtgcagccaggaggttctctgagactgagctgtgccgcttctggttacacatattctagcgggtgcatgggctggttccgccaggctcccggcaaggaacgtgagggtgtggcaactatcaattccgacggctctacaaactacgcagattctgttaaaggccgcttcacaatctctaaggacaacgccaaaaacactctgtacttgcagatgaatagcctgaagcctgaagacactgccatgtactattgcgcagctgagccctactgttctggaggctacccccgctggtctgtggccgagttcggttactggggacaaggaacccaggtcacagtgtccagt

SEQ ID NO: 222 - DNA, 372 bp, Artificial Sequence
  Description of Artificial Sequence: Synthetic polynucleotide
caggttcagctccaggagtcaggcgggggtcttgtccagcctggtggctccctgcgcctgtcctgtgctgcctccggttacacctactccagctattgcatgggatggttcagacaagcgccaggcaaggaacgtgagggggtcgcccacattgactccgacggttccactacctacgccgacagcgtcaaaggccgcttcgcgatttctaaggataacgctaagaatactctgtacttgcagatgaactcactgaagccagaggacacggccatgtattactgcgcagccgatccgatccccggccccggctattgtgacggtggcccgaacaagtactggggacagggcacccaagtgacggtgtcctct

SEQ ID NO: 223 - DNA, 378 bp, Artificial Sequence
  Description of Artificial Sequence: Synthetic polynucleotide
caggtacagttgcaggagagcggaggcggttccgtgcaggcaggtggctctcttagactgtcctgcgccgcgagcgggtacacctacagtagctattgtatgggctggttccgccaggctcctggtaagggtcgcgagggcgtcgctgccatcgactccgatggctctactcgctacgcagattctgtcaaggggcgcttcacaatttccaaggacaacgccaagaacacgctttacttgcagatgaactcactgaagccggaggacaccgctatgtattactgcgctgccgagccctactgttctgggggctacaagcgcactatggtggccgagttcggatattggggccagggtacacaggtgaccgtcagttct

SEQ ID NO: 224 - DNA, 372 bp, Artificial Sequence
  Description of Artificial Sequence: Synthetic polynucleotide
caggtgcagttgcaggagtctggcggtggctctgtgcaggctgggggctctctgcgcctgagttgcgctgccagcggttacacctactccagctattgtatgggatggttccgccaggctccggggaaggagagggagggcgtggcccatatcgactctgatggctccacatcctacgccgacagcgtgaagggacgtttcaccattagcaaggacaatgcgaagaataccctctacttgcagatgaactccctgaagccggaggatactgccatgtattactgcgccgctgatcccatcccagggcctgggtactgtgacggaggcccgaacaagtattggggacaaggaacgcaggtcacagtgtcatct

SEQ ID NO: 225 - DNA, 372 bp, Artificial Sequence
  Description of Artificial Sequence: Synthetic polynucleotide
caggtacaactccaggagagtggtggaggctccgttcaagccgggggctccctgcggctgtcctgtgcggccagcggttacacctattcatcttactgtatgggctggttccggcaggcccctggtaaggaaagagagggtgtcgctcacattgattccgacggtagtacctcttacgcagactctgtcaagggcaggttcaccatctctaaggacaatgccaagaacaccttgtacctccagatgaactctctgaagcccgaggacactgcaatgtactattgtgcggctgaccctattcccggccctggatattgcgacggcggacctaacaattactggggacagggcacccaggtcaccgtcagctcc

SEQ ID NO: 226 - DNA, 378 bp, Artificial Sequence
  Description of Artificial Sequence: Synthetic polynucleotide
caggttcagctccaagaatccggcgggggctctgtgcaggcgggcggaagtctgcgtctgtcatgcgctgccagcgggtacacttactcttccggttgtatgggctggtttaggcaggctccgggaaaggaaagggagggcgtcgcaactatcaacagcgacggctctacgaactacgctgactctgtgaaaggccgcttcaccatcagcaaagacaacgccaaaaatacactgtatctccagatgaatagcttgaaacccgaggacaccggaatgtattactgcgcggcagagccatactgttcaggcggttacccaagatggtccgtggctgagttcggttattgggggcagggcactcaggttactgtgtcttcc

SEQ ID NO: 227 - DNA, 375 bp, Artificial Sequence
  Description of Artificial Sequence: Synthetic polynucleotide
caggtgcagctccaggaatccgggggcggttctgtgcaggctggtggctctctgcgcctgtcttgcactgtttccaggtacactgcctctgtaaactatatgggctggtttagacaagctccgggcaaggaacgcgaaggcgtcgctaccatctttacaggtgcaggtacgacctattacgccaatagcgttaaagggaggttcaccatctccagggacaatgccaaaaacacagcctatctccagatgaactccctcaaacctgaagacacagccatctactattgcgcggttgacttccgtggtggcctgctctatagaccggcgtatgagtacacctaccgtggacaaggcacccaagtcacagtgagcagc

SEQ ID NO: 228 - DNA, 378 bp, Artificial Sequence
  Description of Artificial Sequence: Synthetic polynucleotide
caggtgcagctccaagagtccggcggagggagtgtagaggctggcgggtccctgcgccttagctgcgcggccagcggctatacacacagttcttattgtatgggttggttccgccaagctccgggaaaggagcgtgagggcgtggctgccatcgacgtggatggctccacaacctacgccgacagcgtgaagggcaggtttacgatctctaaggataacgctaagaatactctctatttgcagatgaactccctcaaacccgaggatacaggaatgtactattgcgctgccgagttcgccgactgctcaagcaattatttcctgcctccaggagccgttaggtactggggccaggggactcaggtcacagtaagcagc

SEQ ID NO: 229 - DNA, 375 bp, Artificial Sequence
  Description of Artificial Sequence: Synthetic polynucleotide
caggtgcagctccaggagagcggtggcggatcagtgcaggctggaggctccctcagactgtcctgcaccgtgagccgctataccgcctccgtcaactatatgggatggtttaggcaggctccgggcaaggagcgcgagggggtcgcgactatcttcaccggagccggtactacctattacgctaattctgttaaaggccgctttaccattagtcgcgacaacgctaagaacacagcttacctccagatgaactctctgaagccagaggataccgccatgtattactgcgccgtggacttccggggcggtttgctctaccgcccggcctacgaatacacctatcgcggccagggcacgcaggtcacggtgtcctca

SEQ ID NO: 230 - DNA, 378 bp, Artificial Sequence
  Description of Artificial Sequence: Synthetic polynucleotide
caggtgcagctccaagagtccggtggaggcagcgtccaggccgggggtagtcttaggctcagctgtgctgccagtggagacacctactcttcctattgcatgggatggttcagacaggcccccggcaaagagcgcgagggcgttgcattcatcgactccgacggctccactcgctacgccgatagcgtggagggccgttttaccatctccaaggacaacgcgaagaacactctgtatctgcaaatgaactccctgaagcccgaagacaccgccatgtactattgcgcggctgagccatactgtagtggcggatatcatcgtaaggaaatggcagagttcggctattggggccagggcacccaggtcactgtgagttcc

SEQ ID NO: 231 - DNA, 372 bp, Artificial Sequence
  Description of Artificial Sequence: Synthetic polynucleotide
caggtgcagttgcaggaatccggcggaggctctgtgcaggcgggcggttccctccgcctgagttgtgccgcgtctggctatacttactcttcctattgtatgggatggttccggcaagcgcccggcaaagagcgggagggcgttgcgcatatcgacagtgatggtagcaccagttacgctgatagcgtgaaaggcagattcactatctcaaaggataacgcgaagaacactctttacctccagatgaactcccttaaacctgaggataccgcgatgtattactgtgctgccgaccccattcccggccctggatactgtgacggaggccctaacaagtaccgtgggcaaggaacacaggtcacagtgtccagc

SEQ ID NO: 232 - DNA, 372 bp, Artificial Sequence
  Description of Artificial Sequence: Synthetic polynucleotide
caggtgcaactccaggagtctggcgggggcagcgtccaggcaggtggaagtctccgtctctcatgtgctgccagcggctatacatactccagctactgtatgggatggtttagacaggcacccggcaaggagcgcgaaggggtggcccatatcgactccgatggcagcacaacctatgccgactctgtgaaagggcggttcgccatctccaaggacaacgctaagaataccctgtacctccagatgaactctctgaagcctgaggacaccgccatgtattactgcgctgccgacccaatccctggcccaggttactgcgatgggggaccaaacaaatattggggacagggcacgcaggttacagtctccagc

SEQ ID NO: 233 - DNA, 363 bp, Artificial Sequence
  Description of Artificial Sequence: Synthetic polynucleotide
caggtccaactccaggaaagtggaggtggctctgttcaggccgggggcagcctgaggctgagctgcaccggctcaggctatacagccagtaataactgcatgggctggttccgtcaagcgcccggcaaagagcgtgaaggtgtggccgtaatttttaccggcgctggcaccagctattacgacagttccgtgggccgtctgttcatcagctcacaggacgccgcttccaccctcgatcagttgctgatgagccttctgcccgatgacaccgcagtaatgtactgtggagccgaagatgactgcacactgctcctgatgacgccaaaccccgatgaccaatggtcccgcctgagtgtgtcctcc

SEQ ID NO: 234 - DNA, 372 bp, Artificial Sequence
  Description of Artificial Sequence: Synthetic polynucleotide
caggtgcagctccaggagagcgggggcggttctgttcaggcgggaggcagcctgcgtctgtcctgtgcagcctctggttacacagacagtcgttactgcatgggctggttccgcaaggcacctggaaaggagcgcgagggtgttgcgcacatcgactccgacgggagcactagctatgctgacagcgtgaaggggcgcttcactatcagcaaggataacgcgaaaaacaccttgtaccttcagatgaactccctcaaacccgaagacacagcgatgtactattgtgccgctgatccgatcccagggcctggctactgtgatggtggacctaataagtactgggggcagggaactcaggtgaccgtgtcatca

SEQ ID NO: 235 - DNA, 378 bp, Artificial Sequence
  Description of Artificial Sequence: Synthetic polynucleotide
caggtccagttgcaggaatctggaggcggttccgtgcaagcagggggctcactcagactgtcctgcgctgccagcggctacacttactcttcatattgcatgggctggttccgccaggcaccgggcaaggagcgggaaggcgtggccgctattgatagcgatggctctacgcgctacgcagatagcgtgaaagggaggttcacgatctccaaagataatgccaagaaaattctgtatctccagatgaactctctgaaggtcgaggacaccgccatgtactattgtgcagccgaaccctactgttctggtggctacaagaggactatggtggccgagttcggcttctggggccaggggacccaagtgactgtcagtagc

SEQ ID NO: 236 - DNA, 372 bp, Artificial Sequence
  Description of Artificial Sequence: Synthetic polynucleotide
caggtgcaacttcaggagagcggtggcggatctgtgcaggctggagggtctctgaagctgtcctgcgcggccagcggttacacatacagtagctactgcatgggatggtttcgtcaggccccaggcaaggagcgcgaaggagtggcgcacatcgactccgatgggtccaccacatacgccgactccgtgaagggccgtttcacaatcagcaaggataacgcgaagaacacgctgtacttgcagatgaactctctcaaaccagaggacactgcaatgtactattgcgcggctgaccccatccctggccctggttactgtgacggtggccccaacaattactgggggcaagggacccaagtcaccgtgtcctcc

SEQ ID NO: 237 - DNA, 372 bp, Artificial Sequence
  Description of Artificial Sequence: Synthetic polynucleotide
caggtccagctccaggagtccggcgggggctccgtccaggcagggggctccctgcgtctgtcatgcgccgcttctgggtatacctacagttcctattgtatgggttggtttcgccaagcacccggtaaggagcgcgaaggtattgcgcacattgatagcgatggctccacaacctatgctgacagtgtgaaaggacgcttcactatttccaaggataacgctaagaacacactctaccttcagatgaacagcctgaagccggaagacaccgcaatgtactattgtgcagctgaccccattcctggacccggttactgtgatggaggtcctaataactattggggacagggcactcaagtgaccgtctcaagc

SEQ ID NO: 238 - DNA, 372 bp, Artificial Sequence
  Description of Artificial Sequence: Synthetic polynucleotide
caggtgcagttgcaggagagcgggggtggctctgtgcaggccgggggctccctgaggctgagctgcgcggccagcgggtacagctactctagctattgcatgggttggttccgccaggcccctggcaaggagcgcgagggagtggccacgattgactcagatggcatgacccgttatgcggattccgtcaaggggcgcttcaccatcagcaaagataacgccaaaaataccctgtacttgcagatgaactcactgaaacctgaggatacagccatgtattactgcgcagctccgctctatgactgtgactctggtgccgtgggtagaaacccaccttactgggggcagggaacccaggtgaccgtgtcctca

SEQ ID NO: 239 - DNA, 366 bp, Artificial Sequence
  Description of Artificial Sequence: Synthetic polynucleotide
caggtccagctccaggaaagcggtgggggcagcgtccaaacagggggtagcctgcgcctctcttgcgcagccagcggctacacatatctgcgcggatgtatgggctggttccgccaggcccctggtaaggaaagagagggggtggccgtgatggacgtggttggagacagacgttcctacattgattccgtgaagggccgctttactatctcacgcgataacgcggctaactctgtgtatttgcagatggataacctgaagcccgaggacaccgctatgtactattgcacagctggtcccaactgtgtcggttggcgctccggcctggactattggggtcagggaacccaggttacagttagcagt

SEQ ID NO: 240 - DNA, 381 bp, Artificial Sequence
  Description of Artificial Sequence: Synthetic polynucleotide
caggtgcagctccaggagagtggtggcggttctgtccaagctggcggagccctgcgcctgtcctgcacagcaagcggctacaccgcctctagcatttgcatgggatggttccgtcaggccccaggcaaggagagggagagagtggctgtgattaccacggcagcctccggtacttactatgccgactctgtgaatggccgcttctcaatctctcagaataacgccaaaaatactgtgtacctccagatgaactccctgaaacctgacgataccgcgatgtattactgcgcagccacccggcgcggcggtgactgcctggacccattgcagaccccagcctataatacctggggccagggaacccaggtcaccgtctcttct

SEQ ID NO: 241 - DNA, 369 bp, Artificial Sequence
  Description of Artificial Sequence: Synthetic polynucleotide
caggtgcagctccaggaaagcggcggtggctccgtccaggccggtggctccctgaggctgagctgtgtggcttccggcgatacttattctcgcaagtacatcgcatgggtgcgtcaggtgcccggtaaagaacgtgagggagtggcagtgatgtataccccaggctccgctacttactatacagacacagtgatgggtcgtttcaccatctcccaggacaacgccaagaacactgtgtaccttcaaatgaacagcctcaaacctgaagacaccgccatgtacttttgcgcggccaaggccagcggctccatgtttaacttccgcgattacacttattggggacagggcactcaggtgaccgtaagctct

SEQ ID NO: 242 - DNA, 363 bp, Artificial Sequence
  Description of Artificial Sequence: Synthetic polynucleotide
caggtgcagctgcaagaaagcggaggtggctctgtccaggcaggaggctccctccggcttagctgcgctaccagcgggtatgcttcctgttcccgcgccatgaggtggtacaggcaggcaccgggcaaggagcgcgaatttgtggcgtacatcgacggggtgggcagtactggttatgcggacagcgttaaaggccggtttaccatctcccaagataatgcaaagtacacggcttacttgcagatgaactccctcaagcctgaggataccgcgatgtattactgtaatcggggctgtagagccgatggtagcaatagtctggacaactactggggccagggcacacaggtgactgtctcttca

SEQ ID NO: 243 - DNA, 375 bp, Artificial Sequence
  Description of Artificial Sequence: Synthetic polynucleotide
caggtgcagttgcaggagtccggcggtggcagcgttcaggcgggcggtagcctgcgtctgagctgcgccgcgtccggctacacctataaccgtcgcttcatgggttggttccgtcaagcgcccggcaaggagagagagggcctcgccattatctacacccccaacagctccaccttctacgccgactctgtgacgggccgctttacaatctcacaggattctgcccgcaacaccgtctatttgcagatgaactccctgaaacctgaggacaccgctatgtactattgtgcagccgctcgcatcgcttctatgactgagctttcagtgagagatatggactattggggcaagggcacccaggtgaccgtttcctcc

SEQ ID NO: 244 - DNA, 378 bp, Artificial Sequence
  Description of Artificial Sequence: Synthetic polynucleotide
caggtacaactccaggagagcgggggaggtagcgtacaggctggcgggtccttgcgtctgagctgcactgcatctcgttacatcgctcttaatgcgtgtatggcttggattcggcaggcccccggctccgaaagggaggtcgtggccacaatcgtgactgatggctccagaacctattacgcagactctgtcaagggccggtttactatctctcaagacaacgccaagaacaccatgtacctccagatgaacggtttgaaacccgaagacaccgccatgtattactgtgcagccgacaggcgctgccccgtgtccagagccccatacgaatacgaactgcgctactggggtcagggcacccaggtgactgtcagcagc

SEQ ID NO: 245 - DNA, 369 bp, Artificial Sequence
  Description of Artificial Sequence: Synthetic polynucleotide
caagtccagcttcaagaaagcggagggggctctgttcaggcaggcgggtccctccggctgtcctgcgctgcctccggctacacatacaacggaaagtgcatggcttggttccgccaggctcccggcaaggagcgcgaagtcgtggctggcatttacaccgggggctccagcacatattacgccgatagtgtgaagggacgctttacgatttcccaagacaatgctaaaaatacagtctatctccagatggacagcctgaagcccgaagacactgccatgtattactgcgccaccagcagaagctgtagcgacctgcgcagacgctccatcgcctactggggacaggggactcaggtcaccgtcagctct

SEQ ID NO: 246 - DNA, 381 bp, Artificial Sequence
  Description of Artificial Sequence: Synthetic polynucleotide
caggtccagttgcaagagagtggtggcggatcagtacaggctgggggcagtctgcgcctctcttgtgcttcctctggctatatctcctctagttactgtatggcctggttccgtcaggctcccggtaaagagcgtgagggtgctgcgggcgtgaccagagacggcaagacctattacggcgactctgtaaagggccggttcgcgatctctcgcgacaacgctaagaacactttgtatctccagatgaacagcctgaaacccgaggacaccgctatgtattactgtgccgcaggccctccgccttgcatcacctccatgcctgcgggcggagactatggttaccgctactggggccagggaacacaggtgactgtgtcctcc

SEQ ID NO: 247 - DNA, 348 bp, Artificial Sequence
  Description of Artificial Sequence: Synthetic polynucleotide
caggtgcaacttcaggagagcggtggaggtctggtccaaccaggaggctcactccgcctgtcctgcgcggccagtggttttactttttctaactacggcatgtcttgggtgcgccaggcaccaggcaagggcctggagtgggtaagcgggatcaatagtggcggagatgacaccttctacacggacagcgtgaagggccgcttcactatcagtagggataacgctaagaacactctctacttgcagatgaactccctgaagaccgaagacaccgccatgtattactgtgctatgggagccagtgggatgatccctcgcggtcagggcacccaaatcactgtcagttct

SEQ ID NO: 248 - DNA, 345 bp, Artificial Sequence
  Description of Artificial Sequence: Synthetic polynucleotide
caagtacagctccaggagagtggcggtgggctcgtgcaacccggaggttccctgagattgtcctgtgtggcctctggttttaccttctctgattacgccatgagctgggtgcgccaagctccaggaatgggattggaacgggtctctgcaatcggtcgcgacggctccactttctaccctgactctgtgaaaggccgcttcacaatctctcgcgataacgccaagaataccctgtacctccagctgaacagtttgaagaccgaagatactgctatgtattactgtgccaaagaagagccaggttcctcttcacgcggacagggaacccaggtgacagtctcttcc

SEQ ID NO: 249 - DNA, 381 bp, Artificial Sequence
  Description of Artificial Sequence: Synthetic polynucleotide
caggtgcagcttcaggagtctggtgggggcagcgtgcagctgggtggcagtctgcgtctgagttgtctggtgagtggcagtactgacaacatcaagtacatgggctggtttcgccaggcccctggcaaagagcgcgaaggagtggccgctgtgtatacgtccggcggtgcggttgtgtacgccgatagtgtgaagggcaggttcaccattagtcaggatgacgctaagaacaccatgtacctccagatgaactccctgaagccagaagataccgctatgtactattgcgctgcgtcccgtgctcccgcaccccctcgccttctgttgcagcgggcgctggtggaatattggggccaggggacccaggtgaccgtcagtagc

SEQ ID NO: 250 - DNA, 363 bp, Artificial Sequence
  Description of Artificial Sequence: Synthetic polynucleotide
caggtccagttgcaagagagcggtgggggcctggttcagcctggtgggagcctgcgtctctcctgtgccgcttctggcttcaccttttctaacgccacaatgagctgggtccgccaagcgccgggtaaagaaatcgaatgggtcagtgcaatctcaaacagcagaggcacgaaatattacgcagccttcgtcaaggggcgcttcacgatttcccgcgataatgctaaaaatacactgtacctccagcttaataacctgaagaccgaggacaccgcaatgtactattgtactaaggactggaaaacaagctattctgactatgacctgtctgatggccaaggcactcaggtgaccgtcagtagc

SEQ ID NO: 251 - DNA, 345 bp, Artificial Sequence
  Description of Artificial Sequence: Synthetic polynucleotide
caggtccagctccaggaaagcgggggcggtttggtgcagcccggtgggagcctgagactgagctgtgctgcctctggctttaccttttccgactacgccatgtcctgggtccgtcaggcccccggaatgggtctggagagagtgtctgccatcggcagggacggctctaccttctacccggattccgtaaaaggccgcttcaccatctcccgtgacaacgcgaagaacacactgtacttgcagctcaactccctgaagactgaagacaccgcgatgtattactgcgctaaggaagagccaggctcttcaagtagaggccagggtactcaggtgacagtgtctagc

SEQ ID NO: 252 - DNA, 348 bp, Artificial Sequence
  Description of Artificial Sequence: Synthetic polynucleotide
caggtgcagcttcaggagtccgggggaggcctggtccagcccggtggctcactgcgcctgtcttgtgccgcttccggctttaccttctctaactacggcatgagctgggttaggcaggcacccggcaaaggtctggagtgggtgtctggtatcaattctggaggcgatgacacattttacacagattcagtcaagggccgcttcactatctccagggataacgctaagaacactctgtacctccaaatgaactccctgaagacggaagacaccgctatgtactattgcgcgatgggcgcttccggtatgatcccgcgcggacaaggcacccaggtgactgtaagttct

SEQ ID NO: 253 - DNA, 387 bp, Artificial Sequence
  Description of Artificial Sequence: Synthetic polynucleotide
caggtacagctccaggaaagtggcggtggctccgtccaggcgggcggaagcctgcggctgtcctgcgccgtgtcccgctatacaattagccggtcagattgtatgggctggttccgtcaagccccagggaaggagagggagggtgtcgcccgtatcggcagcgacggcacgacaagctatgcggactccgtcaaggagcgttttaccatctctaaggacaacgcaaagaacatcctgtacctccagatgaacagtctcaagcccgaggacactgcccgttattactgtgctgccaccgccctgcttctgggaagaggctcagcgtgtcacaaagaggtgtcagtgttctcttggtggggccagggcacccaggtgactgtgtcttcc

SEQ ID NO: 254 - DNA, 348 bp, Artificial Sequence
  Description of Artificial Sequence: Synthetic polynucleotide
caggtccaactccaggagtctggcgggggcctggtccagccaggaggctccctccgtctctcctgcgccgcttccggcttcaccttcagtaattacggcatgagttgggttcgccaggctcccggcaagggcctggagtgggtctcaggtatcaattctgggggcgacgatacattctatacagactccgttaagggccgctttacgattagccgcgataacgtgaagaataccctttatctccagatgaactccctgaagaccgaagatactgcgatgtattactgcgcaatgggggcctccggtatgattcctagaggccagggcacccaagtgaccgtcagcagt

SEQ ID NO: 255 - DNA, 387 bp, Artificial Sequence
  Description of Artificial Sequence: Synthetic polynucleotide
caggtgcagctccaggaaagtggagggggctctgtccaggcaggcggtagtctgcgcctctcctgcgcctctagcggttacatttcctctcgctctacctactgtatgggatggttccgccaggctccgggcaaggaacgcgaggtggcggcagttgtgaccggggactctcgtacctactatggtgactcagtgaagggccgctttgcgattagtcgcgacaatgcgaagaacaccctgtacctccagatgaactctctgaagcctgaggacactgccatgtactattgcgcggctggaccccctccctgcattaccactatgcccgctgggggtgactacgggtatcggtattggggtcaaggcacccaggtgacagttagcagc

SEQ ID NO: 256 - DNA, 372 bp, Artificial Sequence
  Description of Artificial Sequence: Synthetic polynucleotide
caggtgcagttgcaggagtctggcggaggctccgtgcagtccggcgggagcttgcgcctctcttgcgctgcctctggattcacgtactcaagctactgtatgggctggtttcgccaagcgccaggtaaggaacgcgaaggcgtggcagccattgattccgacggttccacttcttatgctgacagcgtcaagggtagattcactatctctaaggacaacgctaagaacaccctgtacctccagatgaactccctgagacctgaagataccgctatgtattactgtgcggcagacggcgagtataacgattacgtttgctggagcactggtcttcggtatcggggacagggtacacaggtgaccgtgagcagt

SEQ ID NO: 257 - DNA, 387 bp, Artificial Sequence
  Description of Artificial Sequence: Synthetic polynucleotide
caggtccagttgcaggagtcaggaggtggctccgtgcaagccgggggctcccttcgcctgtcttgcgctagtagcggatacatcagttcccgctccacatattgtatgggctggttccgtcaagcgcccggcaaagagcgcgaggtggcggcaatcgtgacgggtgattccaggacctactatggtgacagcgtgcgcggtcgttttgccatcagccgggataacgcgaagaatacactttacctccagatgaatagcctgaagccagaggataccgccatgtattactgtgccgctgggcctcccccttgtatcacatcaatgcctgctgggggcgattacggctacagatactggggtcaggggacccaggtgaccgtgtcttca

SEQ ID NO: 258 - DNA, 363 bp, Artificial Sequence
  Description of Artificial Sequence: Synthetic polynucleotide
caagtgcagctccaggagtccggtggcgggctggtgcagcctggcagttccctgcgcctgtcctgcgcggccagtggattcaccttctccaacgctactatgtcttgggtccgccaagctcctgggaaggagatcgaatgggtgtctgcaatctctaatagcaggggaaccaagtactatgcggctttcgtgaaggggcgtttcaccatctctcgtgacaacgccaaaaacaccttgtacctgcaactgaacaatctgaaaaccgaagataccgccatgtattactgcactaaagattggaaaacgtcctactccgattacgatctgagtgatggccagggaactcaagtgaccgtctctagc

SEQ ID NO: 259 - DNA, 384 bp, Artificial Sequence
  Description of Artificial Sequence: Synthetic polynucleotide
caggtgcagctccaggaaagtggaggcgggagcgtgcaggcaggcgggtcactcagactgtcttgtgcgtctagcgggtatatctctcgtagctcctattgcatgggatggtttcgccaggctccaggaaaggaacgtgaagttgccgctatcgtgacaggtgacggacgtacctattacggcgactctgtcaagggccgcttcgcgatcagccgtgataatgccaagaacaccctttatttgcagatgaacagtctgaagcccgaggatactgctatgtactattgtgtggctggacccccaccttgcatcaccactatgccagccggtggcgactatggatacaggtactggggacgcggcacccaggtcacagtctctagc

SEQ ID NO: 260 - protein, 6 aa, Artificial Sequence
  Description of Artificial Sequence: Synthetic 6xHis tag
HHHHHH

SEQ ID NO: 261 - protein, 8 aa, Artificial Sequence
  Description of Artificial Sequence: Synthetic 8xHis tag
HHHHHHHH

SEQ ID NO: 262 - protein, 800 aa, Artificial Sequence
  Description of Artificial Sequence: Synthetic polypeptide
  SITE (1)..(20): This region may encompass 1-20 residues
  SITE (21)..(40): This region may encompass 1-20 residues
  SITE (41)..(60): This region may encompass 1-20 residues
  SITE (61)..(80): This region may encompass 1-20 residues
  SITE (81)..(100): This region may encompass 1-20 residues
  SITE (101)..(120): This region may encompass 1-20 residues
  SITE (121)..(140): This region may encompass 1-20 residues
  SITE (141)..(160): This region may encompass 1-20 residues
  SITE (161)..(180): This region may encompass 1-20 residues
  SITE (181)..(200): This region may encompass 1-20 residues
  SITE (201)..(220): This region may encompass 1-20 residues
  SITE (221)..(240): This region may encompass 1-20 residues
  SITE (241)..(260): This region may encompass 1-20 residues
  SITE (261)..(280): This region may encompass 1-20 residues
  SITE (281)..(300): This region may encompass 1-20 residues
  SITE (301)..(320): This region may encompass 1-20 residues
  SITE (321)..(340): This region may encompass 1-20 residues
  SITE (341)..(360): This region may encompass 1-20 residues
  SITE (361)..(380): This region may encompass 1-20 residues
  SITE (381)..(400): This region may encompass 1-20 residues
  SITE (401)..(420): This region may encompass 1-20 residues
  SITE (421)..(440): This region may encompass 1-20 residues
  SITE (441)..(460): This region may encompass 1-20 residues
  SITE (461)..(480): This region may encompass 1-20 residues
  SITE (481)..(500): This region may encompass 1-20 residues
  SITE (501)..(520): This region may encompass 1-20 residues
  SITE (521)..(540): This region may encompass 1-20 residues
  SITE (541)..(560): This region may encompass 1-20 residues
  SITE (561)..(580): This region may encompass 1-20 residues
  SITE (581)..(600): This region may encompass 1-20 residues
  SITE (601)..(620): This region may encompass 1-20 residues
  SITE (621)..(640): This region may encompass 1-20 residues
  SITE (641)..(660): This region may encompass 1-20 residues
  SITE (661)..(680): This region may encompass 1-20 residues
  SITE (681)..(700): This region may encompass 1-20 residues
  SITE (701)..(720): This region may encompass 1-20 residues
  SITE (721)..(740): This region may encompass 1-20 residues
  SITE (741)..(760): This region may encompass 1-20 residues
  SITE (761)..(780): This region may encompass 1-20 residues
  SITE (781)..(800): This region may encompass 1-20 residues
  SITE (1)..(800): This sequence may encompass 1-20 “(Gly)m(Ser)o” repeating units, wherein m = 1 to 20, o = 1 to 20
  See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 263 - protein, 100 aa, Artificial Sequence
  Description of Artificial Sequence: Synthetic polypeptide
  SITE (1)..(100): This sequence may encompass 1-20 “Gly Ser Gly Gly Ser” repeating units
  See specification as filed for detailed description of substitutions and preferred embodiments
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 264 - protein, 1200 aa, Artificial Sequence
  Description of Artificial Sequence: Synthetic polypeptide
  SITE (1)..(20): This region may encompass 1-20 residues
  SITE (21)..(40): This region may encompass 1-20 residues
  SITE (41)..(60): This region may encompass 1-20 residues
  SITE (61)..(80): This region may encompass 1-20 residues
  SITE (81)..(100): This region may encompass 1-20 residues
  SITE (101)..(120): This region may encompass 1-20 residues
  SITE (121)..(140): This region may encompass 1-20 residues
  SITE (141)..(160): This region may encompass 1-20 residues
  SITE (161)..(180): This region may encompass 1-20 residues
  SITE (181)..(200): This region may encompass 1-20 residues
  SITE (201)..(220): This region may encompass 1-20 residues
  SITE (221)..(240): This region may encompass 1-20 residues
  SITE (241)..(260): This region may encompass 1-20 residues
  SITE (261)..(280): This region may encompass 1-20 residues
  SITE (281)..(300): This region may encompass 1-20 residues
  SITE (301)..(320): This region may encompass 1-20 residues
  SITE (321)..(340): This region may encompass 1-20 residues
  SITE (341)..(360): This region may encompass 1-20 residues
  SITE (361)..(380): This region may encompass 1-20 residues
  SITE (381)..(400): This region may encompass 1-20 residues
  SITE (401)..(420): This region may encompass 1-20 residues
  SITE (421)..(440): This region may encompass 1-20 residues
  SITE (441)..(460): This region may encompass 1-20 residues
  SITE (461)..(480): This region may encompass 1-20 residues
  SITE (481)..(500): This region may encompass 1-20 residues
  SITE (501)..(520): This region may encompass 1-20 residues
  SITE (521)..(540): This region may encompass 1-20 residues
  SITE (541)..(560): This region may encompass 1-20 residues
  SITE (561)..(580): This region may encompass 1-20 residues
  SITE (581)..(600): This region may encompass 1-20 residues
  SITE (601)..(620): This region may encompass 1-20 residues
  SITE (621)..(640): This region may encompass 1-20 residues
  SITE (641)..(660): This region may encompass 1-20 residues
  SITE (661)..(680): This region may encompass 1-20 residues
  SITE (681)..(700): This region may encompass 1-20 residues
  SITE (701)..(720): This region may encompass 1-20 residues
  SITE (721)..(740): This region may encompass 1-20 residues
  SITE (741)..(760): This region may encompass 1-20 residues
  SITE (761)..(780): This region may encompass 1-20 residues
  SITE (781)..(800): This region may encompass 1-20 residues
  SITE (801)..(820): This region may encompass 1-20 residues
  SITE (821)..(840): This region may encompass 1-20 residues
  SITE (841)..(860): This region may encompass 1-20 residues
  SITE (861)..(880): This region may encompass 1-20 residues
  SITE (881)..(900): This region may encompass 1-20 residues
  SITE (901)..(920): This region may encompass 1-20 residues
  SITE (921)..(940): This region may encompass 1-20 residues
  SITE (941)..(960): This region may encompass 1-20 residues
  SITE (961)..(980): This region may encompass 1-20 residues
  SITE (981)..(1000): This region may encompass 1-20 residues
  SITE (1001)..(1020): This region may encompass 1-20 residues
  SITE (1021)..(1040): This region may encompass 1-20 residues
  SITE (1041)..(1060): This region may encompass 1-20 residues
  SITE (1061)..(1080): This region may encompass 1-20 residues
  SITE (1081)..(1100): This region may encompass 1-20 residues
  SITE (1101)..(1120): This region may encompass 1-20 residues
  SITE (1121)..(1140): This region may encompass 1-20 residues
  SITE (1141)..(1160): This region may encompass 1-20 residues
  SITE (1161)..(1180): This region may encompass 1-20 residues
  SITE (1181)..(1200): This region may encompass 1-20 residues
  SITE (1)..(1200): This sequence may encompass 1-20 “(Gly)m(Ser)o(Gly)m” repeating units, wherein m = 1 to 20, o = 1 to 20
  See specification as filed for detailed description of substitutions and preferred embodiments
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 265 - protein, 2000 aa, Artificial Sequence
  Description of Artificial Sequence: Synthetic polypeptide
  SITE (1)..(20): This region may encompass 1-20 residues
  SITE (21)..(40): This region may encompass 1-20 residues
  SITE (41)..(60): This region may encompass 1-20 residues
  SITE (61)..(80): This region may encompass 1-20 residues
  SITE (81)..(100): This region may encompass 1-20 residues
  SITE (101)..(120): This region may encompass 1-20 residues
  SITE (121)..(140): This region may encompass 1-20 residues
  SITE (141)..(160): This region may encompass 1-20 residues
  SITE (161)..(180): This region may encompass 1-20 residues
  SITE (181)..(200): This region may encompass 1-20 residues
  SITE (201)..(220): This region may encompass 1-20 residues
  SITE (221)..(240): This region may encompass 1-20 residues
  SITE (241)..(260): This region may encompass 1-20 residues
  SITE (261)..(280): This region may encompass 1-20 residues
  SITE (281)..(300): This region may encompass 1-20 residues
  SITE (301)..(320): This region may encompass 1-20 residues
  SITE (321)..(340): This region may encompass 1-20 residues
  SITE (341)..(360): This region may encompass 1-20 residues
  SITE (361)..(380): This region may encompass 1-20 residues
  SITE (381)..(400): This region may encompass 1-20 residues
  SITE (401)..(420): This region may encompass 1-20 residues
  SITE (421)..(440): This region may encompass 1-20 residues
  SITE (441)..(460): This region may encompass 1-20 residues
  SITE (461)..(480): This region may encompass 1-20 residues
  SITE (481)..(500): This region may encompass 1-20 residues
  SITE (501)..(520): This region may encompass 1-20 residues
  SITE (521)..(540): This region may encompass 1-20 residues
  SITE (541)..(560): This region may encompass 1-20 residues
  SITE (561)..(580): This region may encompass 1-20 residues
  SITE (581)..(600): This region may encompass 1-20 residues
  SITE (601)..(620): This region may encompass 1-20 residues
  SITE (621)..(640): This region may encompass 1-20 residues
  SITE (641)..(660): This region may encompass 1-20 residues
  SITE (661)..(680): This region may encompass 1-20 residues
  SITE (681)..(700): This region may encompass 1-20 residues
  SITE (701)..(720): This region may encompass 1-20 residues
  SITE (721)..(740): This region may encompass 1-20 residues
  SITE (741)..(760): This region may encompass 1-20 residues
  SITE (761)..(780): This region may encompass 1-20 residues
  SITE (781)..(800): This region may encompass 1-20 residues
  SITE (801)..(820): This region may encompass 1-20 residues
  SITE (821)..(840): This region may encompass 1-20 residues
  SITE (841)..(860): This region may encompass 1-20 residues
  SITE (861)..(880): This region may encompass 1-20 residues
  SITE (881)..(900): This region may encompass 1-20 residues
  SITE (901)..(920): This region may encompass 1-20 residues
  SITE (921)..(940): This region may encompass 1-20 residues
  SITE (941)..(960): This region may encompass 1-20 residues
  SITE (961)..(980): This region may encompass 1-20 residues
  SITE (981)..(1000): This region may encompass 1-20 residues
  SITE (1001)..(1020): This region may encompass 1-20 residues
  SITE (1021)..(1040): This region may encompass 1-20 residues
  SITE (1041)..(1060): This region may encompass 1-20 residues
  SITE (1061)..(1080): This region may encompass 1-20 residues
  SITE (1081)..(1100): This region may encompass 1-20 residues
  SITE (1101)..(1120): This region may encompass 1-20 residues
  SITE (1121)..(1140): This region may encompass 1-20 residues
  SITE (1141)..(1160): This region may encompass 1-20 residues
  SITE (1161)..(1180): This region may encompass 1-20 residues
  SITE (1181)..(1200): This region may encompass 1-20 residues
  SITE (1201)..(1220): This region may encompass 1-20 residues
  SITE (1221)..(1240): This region may encompass 1-20 residues
  SITE (1241)..(1260): This region may encompass 1-20 residues
  SITE (1261)..(1280): This region may encompass 1-20 residues
  SITE (1281)..(1300): This region may encompass 1-20 residues
  SITE (1301)..(1320): This region may encompass 1-20 residues
  SITE (1321)..(1340): This region may encompass 1-20 residues
  SITE (1341)..(1360): This region may encompass 1-20 residues
  SITE (1361)..(1380): This region may encompass 1-20 residues
  SITE (1381)..(1400): This region may encompass 1-20 residues
  SITE (1401)..(1420): This region may encompass 1-20 residues
  SITE (1421)..(1440): This region may encompass 1-20 residues
  SITE (1441)..(1460): This region may encompass 1-20 residues
  SITE (1461)..(1480): This region may encompass 1-20 residues
  SITE (1481)..(1500): This region may encompass 1-20 residues
  SITE (1501)..(1520): This region may encompass 1-20 residues
  SITE (1521)..(1540): This region may encompass 1-20 residues
  SITE (1541)..(1560): This region may encompass 1-20 residues
  SITE (1561)..(1580): This region may encompass 1-20 residues
  SITE (1581)..(1600): This region may encompass 1-20 residues
  SITE (1601)..(1620): This region may encompass 1-20 residues
  SITE (1621)..(1640): This region may encompass 1-20 residues
  SITE (1641)..(1660): This region may encompass 1-20 residues
  SITE (1661)..(1680): This region may encompass 1-20 residues
  SITE (1681)..(1700): This region may encompass 1-20 residues
  SITE (1701)..(1720): This region may encompass 1-20 residues
  SITE (1721)..(1740): This region may encompass 1-20 residues
  SITE (1741)..(1760): This region may encompass 1-20 residues
  SITE (1761)..(1780): This region may encompass 1-20 residues
  SITE (1781)..(1800): This region may encompass 1-20 residues
  SITE (1801)..(1820): This region may encompass 1-20 residues
  SITE (1821)..(1840): This region may encompass 1-20 residues
  SITE (1841)..(1860): This region may encompass 1-20 residues
  SITE (1861)..(1880): This region may encompass 1-20 residues
  SITE (1881)..(1900): This region may encompass 1-20 residues
  SITE (1901)..(1920): This region may encompass 1-20 residues
  SITE (1921)..(1940): This region may encompass 1-20 residues
  SITE (1941)..(1960): This region may encompass 1-20 residues
  SITE (1961)..(1980): This region may encompass 1-20 residues
  SITE (1981)..(2000): This region may encompass 1-20 residues
  SITE (1)..(2000): This sequence may encompass 1-20 “(Gly)m(Ser)o(Gly)m(Ser)o(Gly)m” repeating units, wherein m = 1 to 20, o = 1 to 20
  See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 266 - protein, 480 aa, Artificial Sequence
  Description of Artificial Sequence: Synthetic polypeptide
  SITE (5)..(24): This region may encompass 1-20 residues
  SITE (29)..(48): This region may encompass 1-20 residues
  SITE (53)..(72): This region may encompass 1-20 residues
  SITE (77)..(96): This region may encompass 1-20 residues
  SITE (101)..(120): This region may encompass 1-20 residues
  SITE (125)..(144): This region may encompass 1-20 residues
  SITE (149)..(168): This region may encompass 1-20 residues
  SITE (173)..(192): This region may encompass 1-20 residues
  SITE (197)..(216): This region may encompass 1-20 residues
  SITE (221)..(240): This region may encompass 1-20 residues
  SITE (245)..(264): This region may encompass 1-20 residues
  SITE (269)..(288): This region may encompass 1-20 residues
  SITE (293)..(312): This region may encompass 1-20 residues
  SITE (317)..(336): This region may encompass 1-20 residues
  SITE (341)..(360): This region may encompass 1-20 residues
  SITE (365)..(384): This region may encompass 1-20 residues
  SITE (389)..(408): This region may encompass 1-20 residues
  SITE (413)..(432): This region may encompass 1-20 residues
  SITE (437)..(456): This region may encompass 1-20 residues
  SITE (461)..(480): This region may encompass 1-20 residues
  SITE (1)..(480): This sequence may encompass 1-20 “(Gly)(Ser)(Gly)(Gly)(Ser)m” repeating units, wherein m = 1 to 20
  See specification as filed for detailed description of substitutions and preferred embodiments
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 267 - protein, 480 aa, Artificial Sequence
  Description of Artificial Sequence: Synthetic polypeptide
  SITE (4)..(23): This region may encompass 1-20 residues
  SITE (28)..(47): This region may encompass 1-20 residues
  SITE (52)..(71): This region may encompass 1-20 residues
  SITE (76)..(95): This region may encompass 1-20 residues
  SITE (100)..(119): This region may encompass 1-20 residues
  SITE (124)..(143): This region may encompass 1-20 residues
  SITE (148)..(167): This region may encompass 1-20 residues
  SITE (172)..(191): This region may encompass 1-20 residues
  SITE (196)..(215): This region may encompass 1-20 residues
  SITE (220)..(239): This region may encompass 1-20 residues
  SITE (244)..(263): This region may encompass 1-20 residues
  SITE (268)..(287): This region may encompass 1-20 residues
  SITE (292)..(311): This region may encompass 1-20 residues
  SITE (316)..(335): This region may encompass 1-20 residues
  SITE (340)..(359): This region may encompass 1-20 residues
  SITE (364)..(383): This region may encompass 1-20 residues
  SITE (388)..(407): This region may encompass 1-20 residues
  SITE (412)..(431): This region may encompass 1-20 residues
  SITE (436)..(455): This region may encompass 1-20 residues
  SITE (460)..(479): This region may encompass 1-20 residues
  SITE (1)..(480): This sequence may encompass 1-20 “(Gly)(Ser)(Gly)(Ser)m(Gly)” repeating units, wherein m = 1 to 20
  See specification as filed for detailed description of substitutions and preferred embodiments
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 268 - protein, 460 aa, Artificial Sequence
  Description of Artificial Sequence: Synthetic polypeptide
  SITE (4)..(23): This region may encompass 1-20 residues
  SITE (27)..(46): This region may encompass 1-20 residues
  SITE (50)..(69): This region may encompass 1-20 residues
  SITE (73)..(92): This region may encompass 1-20 residues
  SITE (96)..(115): This region may encompass 1-20 residues
  SITE (119)..(138): This region may encompass 1-20 residues
  SITE (142)..(161): This region may encompass 1-20 residues
  SITE (165)..(184): This region may encompass 1-20 residues
  SITE (188)..(207): This region may encompass 1-20 residues
  SITE (211)..(230): This region may encompass 1-20 residues
  SITE (234)..(253): This region may encompass 1-20 residues
  SITE (257)..(276): This region may encompass 1-20 residues
  SITE (280)..(299): This region may encompass 1-20 residues
  SITE (303)..(322): This region may encompass 1-20 residues
  SITE (326)..(345): This region may encompass 1-20 residues
  SITE (349)..(368): This region may encompass 1-20 residues
  SITE (372)..(391): This region may encompass 1-20 residues
  SITE (395)..(414): This region may encompass 1-20 residues
  SITE (418)..(437): This region may encompass 1-20 residues
  SITE (441)..(460): This region may encompass 1-20 residues
  SITE (1)..(460): This sequence may encompass 1-20 “(Gly)(Gly)(Gly)(Ser)m” repeating units, wherein m = 1 to 20
  See specification as filed for detailed description of substitutions and preferred embodiments
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 269 - protein, 40 aa, Artificial Sequence
  Description of Artificial Sequence: Synthetic polypeptide
  SITE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 270 - protein, 50 aa, Artificial Sequence
  Description of Artificial Sequence: Synthetic polypeptide
  SITE (1)..(50): This sequence may encompass 1-10 “Gly Gly Gly Ser Gly” repeating units
GGGSGGGGSGGGGSGGGGSGGGGSGGGGSGGGGSGGGGSGGGGSGGGGSG

SEQ ID NO: 271 - protein, 31 aa, Artificial Sequence
  Description of Artificial Sequence: Synthetic polypeptide
  SITE (1)..(30): This region may encompass 1-10 “Gly Gly Ser” repeating units
GGSGGSGGSGGSGGSGGSGGSGGSGGSGGSG

SEQ ID NO: 272 - protein, 40 aa, Artificial Sequence
  Description of Artificial Sequence: Synthetic polypeptide
  SITE (1)..(40): This sequence may encompass 1-10 “Gly Gly Ser Gly” repeating units
GGSGGGSGGGSGGGSGGGSGGGSGGGSGGGSGGGSGGGSG

SEQ ID NO: 273 - protein, 200 aa, Homo sapiens
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 274 - protein, 193 aa, Mus musculus
MLLLLLPLLLAAVLTRTQADPVPRATRLPVEAKDCHIAQFKSLSPKELQAFKKAKDAIEKRLLEKDLRCSSHLFPRAWDLKQLQVQERPKALQAEVALTLKVWENMTDSALATILGQPLHTLSHIHSQLQTCTQLQATAEPRSPSRRLSRWLHRLQEAQSKETPGCLEASVTSNLFRLLTRDLKCVANGDQCV

SEQ ID NO: 275 - protein, 6 aa, Artificial Sequence
  Description of Artificial Sequence: Synthetic His tag
  SITE (1)..(6): This sequence may encompass 3-6 residues
HHHHHH

SEQ ID NO: 276 - protein, 8 aa, Artificial Sequence
  Description of Artificial Sequence: Synthetic peptide
ASHHHHHH

SEQ ID NO: 277 - protein, 10 aa, Artificial Sequence
  Description of Artificial Sequence: Synthetic peptide
GSHHHHHHHH

(19) United States
(12) Patent Application Publication
(10) Pub. No.: US 2023/0391891 A1
(43) Pub. Date: Dec. 7, 2023

(54) IL28A RECEPTOR BINDING SYNTHETIC CYTOKINES AND METHODS OF USE

(71) Applicant: Synthekine, Inc., Menlo Park, CA (US)

(72) Inventors: Robert Kastelein, Menlo Park, CA (US); Deepti Rokkam, Menlo Park, CA (US); Patrick J. Lupardus, Menlo Park, CA (US)

(21) Appl. No.: 18/006,313

(22) PCT Filed: Aug. 5, 2021
(86) PCT No.: PCT/US2021/044841

§ 371 Date: Jan. 23, 2023

Related U.S. Application Data
(60) Provisional application No. 63/061,562, filed on Aug. 5, 2020.
(60) Provisional application No. 63/078,745, filed on Sep. 15, 2020.
(60) Provisional application No. 63/135,884, filed on Jan. 11, 2021.

(51) Int. Cl. C07K 16/46; C12N 15/63; A61P 37/02

(52) U.S. Cl. CPC C07K 16/468; C12N 15/63; A61P 37/02; C07K 2317/31; C07K 2317/569; C07K 2317/565

(57) ABSTRACT

Provided herein are IFNlambdaR1 binding molecules that bind to IL10Rb and IL28RA and comprise an anti-IL28RA sdAb 2 and an anti-IL28RA V_{H}H antibody.

### CROSS-REFERENCES TO RELATED APPLICATIONS

This application is a U.S. National Stage of PCT/US2021/044841, international filing date Aug. 5, 2021, which claims priority to U.S. Provisional Application No. 63/061,562, filed Aug. 5, 2020, U.S. Provisional Application No. 63/078,745, filed Sep. 15, 2020, and U.S. Provisional Application No. 63/135,884, filed Jan. 11, 2021, the disclosures of which are hereby incorporated by reference in their entirety for all purposes.

### SEQUENCE LISTING

The instant application contains a Sequence Listing which has been submitted electronically in ASCII format and is hereby incorporated by reference in its entirety. Said ASCII copy, created on Sep. 15, 2021, is named 106249-1258354-002310PC_SL.txt and is 227,875 bytes in size.

### FIELD OF THE INVENTION

The present disclosure relates to biologically active molecules comprising single domain antibodies that specifically bind to the extracellular domains of the IL10Rb and IL28RA, compositions comprising such single domain antibodies, and methods of use thereof.

### BACKGROUND OF THE DISCLOSURE

Cytokine and growth-factor ligands typically signal through multimerization of cell surface receptors subunits. In some instance, cytokines act as multispecific (e.g., bispecific or trispecific) ligands which facilitate the association of such receptor subunits, bringing their intracellular domains into proximity such that intracellular signaling may occur. The nature of the cytokine determines which receptor subunits are associated to form the cytokine receptor complex. Cytokines thus act to bridge the individual receptor subunits into a receptor complex that results in intracellular signaling.

The intracellular domains of cytokine receptor subunits possess proline rich JAK binding domains which are typically located in the box1/box region of the intracellular domain of the cytokine receptor subunit near the interior surface of the cell membrane. Intracellular JAK kinases associate with JAK binding domains. When the intracellular domains receptor subunits are brought into proximity, typically by the binding of the cognate ligand for the receptor to the extracellular domains of the receptor subunts, the JAKs phosphorylate each other. Four Janus kinases have been identified in mammalian cells: JAK1, JAK2, JAK3 and TYK2. Ihle, et al. (1995) Nature 377(6550):591-4, 1995; O'Shea and Plenge (2012) Immunity 36(4):542-50. The phosphorylation of the JAK induces a conformational change in the JAK providing the ability to further phosphorylate other intracellular proteins which initiates a cascade that results in activation of multiple intracellular factors which transduce the intracellular signal associated with the receptor resulting intracellular responses such as gene transcription, frequently referred to as downstream signaling. In many instances, the proteins which are phosphorylated by the JAKs are members of the signal transducer and activator of transcription (STAT) protein family. Seven members of the mammalian STAT family have been identified to date: STAT1, STAT2, STAT3, STAT4, STAT5a STAT5b, and STAT6. Delgoffe, et al., (2011) Curr Opin Immunol. 23(5):632-8; Levy and Darnell (2002) Nat Rev Mol Cell Biol. 3(9):651-62 and Murray, (2007) J Immunol. 178(5):2623-9. The selective interplay of activated JAK and STAT proteins, collectively referred to a the JAK/STAT pathway, provide for a wide variety of intracellular responses observed in response to cytokine binding.

The human genome encodes for approximately forty different JAK/STAT cytokine receptors. In principle, approximately 1600 unique homodimeric and heterodimeric cytokine receptor pairs could be generated with the potential to signal through different JAK/TYK/STAT combinations (Bazan, Proc Natl Acad Sci USA. 87(18):6934-8, 1990; Huising et al., J Endocrinol. 189(1):1-25, 2006). However, as of the present knowledge, the human genome encodes for less than fifty different cytokine ligands (Bazan, Proc Natl Acad Sci USA. 87(18):6934-8, 1990; Huising et al., J Endocrinol. 189(1):1-25, 2006), limiting the scope of cytokine receptor complexes to those that can be assembled by the natural ligands. Given that interaction of the a cytokine ligand with the extracellular domains of the cytokine receptor subunits determines the composition of receptor subunits in a receptor complex and the intracellular JAK/TYK and RTK enzymes are degenerate, the number of cytokine and growth factor receptor dimer pairings that occur in nature represents only a fraction of the total number of signaling-competent receptor pairings theoretically allowed by the system.

Naturally occurring cytokine ligands mediate a wide variety of cellular response. In some instances, a heteromultimeric cytokine receptor is composed of one or more receptor subunits that is unique to the receptor complex, referred to as “proprietary” subunits, which interact with other receptor subunits that are shared by multiple cytokine receptors, frequently referred to as “common” receptor subunits. For example, the IL7 receptor is a heterodimeric receptor complex of the IL7Ra proprietary subunit and a CD132 subunit which is also referred to as the “common gamma” subunit as it is a shared receptor subunit of multiple cytokine receptor complexes including IL2, IL4, IL19, IL15 and IL21. The relative affinity and kinetic of the interaction of the cytokine for the ECDs of the receptor subunits and the stability of the complex formed in response to cytokine binding mediates the nature and intensity of the intracellular signaling. In some instances, the binding of the cytokine to a the proprietary subunit enhances the formation of the complete receptor where the affinity of the cytokine for the common subunit may be significantly lower when not associated with the proprietary subunit.

The nuances of the interplay between the cytokine ligand and the receptor subunits is a matter of significant scientific investigation. For example, many properties of naturally occurring cytokines suggest their potential utility in the treatment of human disease but such naturally occurring cytokines may also trigger adverse and undesirable effects. In many instances, the disease is associated with a particular cell type which expresses the receptor for the potentially therapeutic cytokine. However, the cytokine receptor is also expressed on other cell types not desired to be targeted for therapeutic intervention. The administration of the native ligand activates both cell types resulting result in undesirable side effects.

To attempt to generate cytokine analogs which provide selective activation of the desired cell types, a variety of engineered cytokine ligands (or components thereof) have been generated so as to selectively modulate their affinity for the extracellular domains of receptor subunits. These efforts have generated cytokine variants been shown to provide partial activity which results in uncoupling of the beneficial properties of the ligand from the undesired effects. See, e.g, Mendoza, et al. (2019) 567:56-60. However, the engineering of such selective cytokines ligand is based on selective modulation of individual amino acid residues at the interface of the ligand and the receptor. This protein engineering approach to modulation of cytokine receptor affinity requires a three dimensional, usually x-ray crystallographic, map of the interation of the receptor and the cytokine to identify the residues of the cytokine that interface with the receptor subunit. Additionally the effects of amino acid substitutions at these interface residues can be highly variable often requiring a significant amount of time consuming trial-and-error to identify the particular amino acid substitutions required to produce the desired activity profile. However, even once the engineered cytokine with the desired signaling profile is achieved, many proteins are highly sensitive to amino acid substitutions result in significant issues for recombinant expression, both in mammalian expression systems and procaryotic systems where such amino acid substitutions can affect protein refolding when expressed in inclusion bodies.

The anti-inflammatory cytokine interleukin-10 (IL-10), also known as human cytokine synthesis inhibitory factor (CSIF), is classified as a type (class)—2 cytokine, a set of cytokines that includes IL-19, IL-20, IL-22, IL-24 (Mda-7), and IL-26, interferons (IFN-α, -β, -γ, -δ, -ε, -κ, -Ω and -τ) and interferon-like molecules (limitin, IL-28A, IL-28B, and IL-29). Human IL-10 is a homodimer with a molecular mass of 37 kDa, wherein each 18.5 kDa monomer comprises 178 amino acids, the first 18 of which comprise a signal peptide, and two cysteine residues that form two intramolecular disulfide bonds.

The IL-10 receptor, a type II cytokine receptor, consists of alpha and beta subunits, which are also referred to as R1 and R2, respectively. Receptor activation requires binding to both alpha and beta. One homodimer of an IL-10 polypeptide binds to alpha and the other homodimer of the same IL-10 polypeptide binds to beta. In addition to forming a subunit of the ILRb receptor complex, the IL10Rb receptor subunit is a component of the IL22, IL26, IL28, and the interferon lambda L1 receptor complexes, IFNL1 variant. The IFNLR1/IL10RB dimer is a receptor for the cytokine ligands IFNL2 and IFNL3 and mediates their antiviral activity. IL10Rb is also known as CDW210B. In contrast to IL10Ra which is expressed primarily on haematopoietic cells, the IL10Rb receptor subunit is expressed ubiquitously. Although the interaction between IL10 and IL10Ra is specific high-affinity interaction, IL-10's association with IL-10Rb is low affinity shared receptor with reports suggesting that the interaction of IL-10 with IL10Ra induces a confirmational change in IL10Rb facilitating its binding to IL10.

Human IL10Rb (hIL10Rb) is expressed as a 325 amino acid pre-protein comprising a 19 amino acid N-terminal signal sequence. Amino acids 20-220 (amino acids 1-201 of the mature protein) correspond to the extracellular domain, amino acids 221-242 (amino acids 202-223 of the mature protein) correspond to the 22 amino acid transmembrane domain, and amino acids 243-325 (amino acids 224-306 of the mature protein) correspond to the intracellular domain. hIL10Rb is referenced at UniProtKB database as entry Q08334. Murine IL10Rb (mIL10Rb) is expressed as a 349 amino acid pre-protein comprising a 19 amino acid N-terminal signal sequence. Amino acids 20-220 (amino acids 1-201 of the mature protein) correspond to the extracellular domain, amino acids 221-241 (amino acids 202-222 of the mature protein) correspond to the 21 amino acid transmembrane domain, and amino acids 242-349 (amino acids 223-330 of the mature protein) correspond to the intracellular domain. mCD132 is referenced at UniProtKB database as entry Q61190.

IL-10 exhibits pleiotropic effects in immunoregulation and inflammation through actions on T cells, B cells, macrophages, and antigen presenting cells (APC). IL-10 is produced by mast cells, counteracting the inflammatory effect that these cells have at the site of an allergic reaction. Although IL-10 is predominantly expressed in macrophages, expression has also been detected in activated T cells, B cells, mast cells, and monocytes. IL-10 can suppress immune responses by inhibiting expression of IL-1α, IL-1β, IL-6, IL8, TNFα, GM-CSF and G-CSF in activated monocytes and activated macrophages, and it also suppresses IFN-γ production by NK cells. IL10 can block NF-κB activity and is involved in the regulation of the JAK-STAT signaling pathway.

Although monoclonal antibodies are the most widely used reagents for the detection and quantification of proteins, monoclonal antibodies are large molecules of about 150 kDa and it sometimes limits their use in assays with several reagents competing for close epitopes recognition. A unique class of immunoglobulin containing a heavy chain domain and lacking a light chain domain (commonly referred to as heavy chain” antibodies (HCAbs) is present in camelids, including dromedary camels, Bactrian camels, wild Bactrian camels, llamas, alpacas, vicunas, and guanacos as well as cartilaginous fishes such as sharks. The isolated variable domain region of HCAbs is known as a VHH (an abbreviation for “variable-heavy-heavy” reflecting their architecture) or Nanobody® (Ablynx). Single domain VHH antibodies possesses the advantage of small size (˜12-14 kD), approximately one-tenth the molecular weight a conventional mammalian IgG class antibody) which facilitates the binding of these VHH molecules to antigenic determinants of the target which may be inaccessible to a conventional monoclonal IgG format (Ingram et al., 2018). Furthermore, VHH single domain antibodies are frequently characterized by high thermal stability facilitating pharmaceutical distribution to geographic areas where maintenance of the cold chain is difficult or impossible. These properties, particularly in combination with simple phage display discovery methods that do not require heavy/light chain pairing (as is the case with IgG antibodies) and simple manufacture (e.g., in bacterial expression systems) make VHH single domain antibodies useful in a variety of applications including the development of imaging and therapeutic agents.

### SUMMARY OF THE DISCLOSURE

The present disclosure provides compositions useful in the pairing of cellular receptors to generate desirable effects useful in treatment of disease in mammalian subjects.

The present disclosure provides compositions comprising at least a first domain that specifically binds to a first receptor subunit and a second domain that specifically binds to a second receptor subunit, such that upon contacting with a cell expressing the first and second receptors, the composition causes the functional association of the first and second receptors, thereby triggering their interaction and resulting in downstream signaling. In some embodiments, the first and second receptors occur in proximity in response to the cognate ligand binding and are referred to herein as “natural” cytokine receptor pairs.

The present disclosure provides cytokine receptor binding molecules that are ligands for a cytokine receptor, the cytokine receptor binding molecule comprising:
(a) a first single domain antibody (sdAb) that specifically binds to the extracellular domain a first subunit of a cytokine receptor; and
(b) a second single domain antibody that specifically binds to extracellular domain of a second subunit of cytokine receptor subunit;

wherein:
the first sdAb and second sdAb are in stable association;
the first and second subunits of the cytokine receptor are dimerized in response to contact with the cognate ligand for the cytokine receptor; and
contacting a cell expressing the first and the second subunits of the cytokine receptor with an effective amount of the cytokine receptor binding molecule results in the intracellular domains of the first and second subunits of the cytokine receptors being brought into proximity and results in intracelluar signaling.

The present disclosure thus provides binding molecules that comprise a first domain that binds to IL10Rb of the IFNlambdaR1 receptor (IFNlambdaR1 or IFNλR1) and a second domain that binds to IL28RA of the IFNlambdaR1, such that upon contacting with a cell expressing IL10Rb of the IFNlambdaR1 and IL28RA of the IFNlambdaR1, the IFNlambdaR1 binding molecule causes the functional association of IL10Rb and IL28RA, thereby resulting in functional dimerization of the receptors and downstream signaling.

In one aspect, the disclosure provides an IFNlambda receptor 1 (IFNlambdaR1) binding molecule that specifically binds to IL10Rb and IL28RA, wherein the binding molecule causes the multimerization of IL10Rb and IL28RA when bound to IL10Rb and IL28RA, and wherein the binding molecule comprises a single-domain antibody (sdAb) that specifically binds to IL10Rb (an anti-IL10Rb sdAb) and a sdAb that specifically binds to IL28RA (an anti-IL28RA sdAb).

In some embodiments, the anti-IL10Rb sdAb is a VHH antibody and/or the anti-IL28RA sdAb is a VHH antibody.

In some embodiments, the anti-IL10Rb sdAb and the anti-IL28RA sdAb are joined by a peptide linker. In some embodiments, the peptide linker comprises between 1 and 50 amino acids. In some embodiments, the peptide linker comprises a sequence of GGGS (SEQ ID NO: 13).

In some embodiments, the anti-IL10Rb sdAb comprises one or more CDRs in a row of Table 2 or Table 3, wherein each CDR independently comprises 0, 1, 2, or 3 amino acid changes relative to the sequence of one or more CDRs in a row of Table 2 or Table 3.

In some embodiments, the anti-IL28RA sdAb comprises one or more CDRs in a row of Table 4 wherein each CDR independently comprises 0, 1, 2, or 3 amino acid changes relative to the sequence in a row of Table 4.

In some embodiments, the IFNlamdaR1 binding molecule comprises an anti-IL10Rb sdAb comprising a CDR1, a CDR2, and a CDR3 in a row of Table 2 or Table 3 and an anti-IL28RA sdAb a CDR1, a CDR2, and a CDR3 in a row of Table 4.

In some embodiments, the binding molecule comprises an anti-IL10Rb sdAb linked to the N-terminus of a linker and an anti-IL28RA sdAb linked to the C-terminus of the linker.

In some embodiments, the binding molecule comprises an anti-IL28RA sdAb linked to the N-terminus of a linker and an anti-IL10Rb sdAb linked to the C-terminus of the linker.

In some embodiments, the anti-IL10Rb sdAb comprises a sequence having at least 90% sequence identity to a sequence of Table 5 or Table 6.

In some embodiments, the anti-IL10Rb sdAb comprises a sequence of Table 5 or Table 6.

In some embodiments, wherein the anti-IL28RA sdAb comprises a sequence having at least 90% sequence identity to a sequence of Table 7.

In some embodiments, wherein the anti-IL28RA sdAb comprises a sequence of Table 7.

In some embodiments, the anti-IL10Rb sdAb comprises a sequence having at least 90% sequence identity to a sequence of Table 5 or Table 6 and the anti-IL28RA sdAb comprises a sequence having at least 90% sequence identity to a sequence of Table 7.

In some embodiments, the anti-IL10Rb sdAb comprises a sequence having at least 90% sequence identity to a sequence of Table 5 and the anti-IL28RA sdAb comprises a sequence having at least 90% sequence identity to a sequence of Table 7.

In some embodiments, each of the anti-IL10Rb sdAb comprises a sequence of Table or Table 6 and the anti-IL28RA sdAb comprises a sequence of Table 7.

In some embodiments, the disclosure provides an isolated nucleic acid encoding the IFNlamdaR1 binding molecule of the disclosure.

In some embodiments, the isolated nucleic acid comprises a sequence having at least 90% sequence identity to a sequence of Table 8 or Table 9 and the anti-IL28RA sdAb comprises a sequence having at least 90% sequence identity to a sequence of Table 10.

In some embodiments, the disclosure provides an expression vector comprising the nucleic acid encoding the IFNlamdaR1 binding molecules of the disclosure.

In some embodiments, the disclosure provides a host cell comprising a nucleic acid encoding the IFNlamdaR1 binding molecules of the disclosure, or an expression vector comprising the nucleic acid encoding the IFNlamdaR1 binding molecules of the disclosure.

In some embodiments, the disclosure provides a pharmaceutical composition comprising the IFNlamdaR binding molecule of the disclosure and a pharmaceutically acceptable carrier.

In some embodiments, the disclosure provides method of treating an autoimmune or inflammatory disease, disorder, or condition or a viral infection in a subject in need thereof, comprising administering to the subject a therapeutically effective amount of an IFNlamdaR1 binding molecule or a pharmaceutical composition of the disclosure.

In some embodiments, the method further comprises administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody.

In some embodiments, the therapeutic antibody is an antibody that binds a protein selected from the group consisting of BLyS, CD11a, CD20, CD25, CD3, CD52, IgEIL12/IL23, IL17a, IL1B, IL4Ra, IL5, IL6R, integrin-α4β7, RANKL, TNFα, VEGF-A, and VLA-4.

In some embodiments, the disease, disorder, or condition is selected from viral infections, heliobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome, psoriasis, psoriatic arthritis, dermatitis (eczema), exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis; seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.

The present disclosure provides binding molecules that are agonists of the IFNlambdaR1 receptor, the binding molecule comprising:
a first single domain antibody (sdAb) that specifically binds to the extracellular domain of IL10Rb of the IFNlambdaR1 (an “anti-IL10Rb sdAb”), and
a second single domain antibody that specifically binds to the extracellular domain of IL28RA of the IFNlambdaR1 (an “anti-IL28RA sdAb”),

wherein the anti-IL10Rb sdAb and anti-IL28RA sdAb are stably associated and wherein contacting a cell expressing IL10Rb and IL28RA with an effective amount of the binding molecule results in the dimerization of IL10Rb and IL28RA and results in intracelluar signaling characteristic of the IFNlambdaR1 when activated by its natural cognate ligand, IFNlambda1. In some embodiments, one or both of the sdAbs is a an scFv. In some embodiments, one or both of the sdAbs is a VHH.

In some embodiments, one sdAb of the bivalent binding molecule is an scFv and the other sdAb is a VHH.

In some embodiments, the first and second sdAbs are covalently bound via a chemical linkage.

In some embodiments, the first and second sdAbs are provided as single continuous polypeptide.

In some embodiments, the first and second sdAbs are provided as single continuous polypeptide optionally comprising an intervening polypeptide linker between the amino acid sequences of the first and second sdAbs.

In some embodiments the bivalent binding molecule optionally comprising a linker, is optionally expressed as a fusion protein with an additional amino acid sequence. In some embodiments, the additional amino acid sequence is a purification handle such as a chelating peptide or an additional protein such as a subunit of an Fc molecule.

The disclosure also provides an expression vector comprising a nucleic acid encoding the bispecific binding molecule operably linked to one or more expression control sequences.

The disclosure further provides recombinant viral and non-viral expression vectors comprising a nucleic acid encoding the IFNlambdaR1 binding molecules of the present disclosure or the CDRs of the IFNlambdaR1 binding molecules of the present disclosure.

The disclosure also provides an isolated host cell comprising the expression vector comprising a nucleic acid encoding the bispecific binding molecule operably linked to one or more expression control sequences functional in the host cell. In some embodiments, the host comprises a recombinant viral or non-viral expression vector comprising a nucleic acid encoding the IFNlambdaR1 binding molecules of the present disclosure or the CDRs of the IFNlambdaR1 binding molecules of the present disclosure.

In another aspect, the disclosure provides a pharmaceutical composition comprising the IFNlambdaR1 binding molecule described herein and a pharmaceutically acceptable carrier.

In another aspect, the disclosure provides a method of treating an autoimmune or inflammatory disease, disorder, or condition or a viral infection in a subject in need thereof, comprising administering to the subject a therapeutically effective amount of an IFNlambdaR1 binding molecule described herein or a pharmaceutical composition described herein.

Several advantages flow from the binding molecules described herein. The natural ligand of the IFNlambdaR1, IFNlambda1, causes IFNlambdaR1 and IL28RA to come into proximity (i.e., by their simultaneous binding of IFNlambda1). However, when IFNlambda1 is used as a therapeutic in mammalian, particularly human, subjects, it may also trigger a number of adverse and undesirable effects by a variety of mechanisms including the presence of IFNlambdaR1 and IL28RA on other cell types and the binding to IFNlambdaR1 and IL28RA on the other cell types may result in undesirable effects and/or undesired signaling on cells expressing IFNlambdaR1 and IL28RA. The present disclosure is directed to methods and compositions that modulate the multiple effects of IFNlambdaR1 and IL28RA binding so that desired therapeutic signaling occurs, particularly in a desired cellular or tissue subtype, while minimizing undesired activity and/or intracellular signaling.

In some embodiments, the IFNlambdaR1 binding molecules described herein are partial agonists of the IFNlambdaR1. In some embodiments, the binding molecules described herein are designed such that the binding molecules are full agonists. In some embodiments, the binding molecules described herein are designed such that the binding molecules are super agonists.

In some embodiments, the binding molecules provide the maximal desired IFNlambda1 intracellular signaling from binding to IFNlambdaR1 and IL28RA on the desired cell types, while providing significantly less IFNlambda1 signaling on other undesired cell types. This can be achieved, for example, by selection of binding molecules having differing affinities or causing different Eₘₐₓ for IFNlambdaR1 and IL28RA as compared to the affinity of IFNlambda1 for IFNlambdaR1 and IL28RA. Because different cell types respond to the binding of ligands to its cognate receptor with different sensitivity, by modulating the affinity of the dimeric ligand (or its individual binding moieties) for the IFNlambdaR1 receptor relative to wild-type IFNlambda1 binding facilitates the stimulation of desired activities while reducing undesired activities on non-target cells.

### BRIEF DESCRIPTION OF THE FIGURES

FIG. 1 of the attached drawings provides a schematic representation of one embodiment of the bivalent binding molecule of the present disclosure comprising a first single domain antibody (1) and a second single domain antibody (3) and a linker (2) depicted as interacting with a cell membrane (10) associated heterodimeric receptor comprising a first receptor subunit comprising an extracellular domain (4), and transmembrane domain (5) and an intracellular domain (6) interaction of a bivalent binding molecule and a second first receptor subunit comprising an extracellular domain (7), and transmembrane domain (8) and an intracellular domain (9) wherein the intracellular domain of the first receptor (6) and the intracellular domain of the second receptor (9) on of a bivalent binding molecule are within a proximal distance (11).

FIG. 2 of the attached drawings provides a schematic representation of two illustrative configurations of bivalent binding molecules of the present disclosure. Panel A provides a schematic representation of an illustrative single polypeptide chain bivalent binding molecule comprising, from amino to carboxy, a first single domain antibody (1) and a second single domain antibody (3) and a linker (2). Panel B provides a schematic representation of a bivalent binding molecule comprising a first single domain antibody (1) and a second single domain antibody (3) and a linker (2) and a knob-into-hole Fc domain, the Fc domain comprising a first subunit which is a Fc knob (13) and a second subunit which is a Fc hole (14) wherein the bivalent binding molecule is covalently linked to an Fc domain subunit via a IgG hinge sequence (12).

FIG. 3 of the attached drawings provides a schematic representations of two illustrative configurations of bivalent binding molecules of the present disclosure. Panel A provides a schematic representation of an illustrative bivalent binding molecule construct comprising two bivalent binding molecules each attached to a subunit of a knob-into-hole Fc domain, the construct comprising two polypeptide chains, the first polypeptide chain comprising, from amino to carboxy, a first single domain antibody (1), a linker (2) and a second single domain antibody (3), a IgG hinge sequence (12) and a Fc knob subunit (13) and a second polypeptide chain comprising, from amino to carboxy, a first single domain antibody (1), a linker (2) and a second single domain antibody (3), a IgG hinge sequence (12) and a Fc hole subunit (14) wherein the first and second polypeptides are in stable associate via the interaction of the knob-into-hole Fc domain. Panel B provides schematic representation of a an alternative arrangement of a bivalent binding molecule construct comprising two polypeptides a first polypeptide chain comprising, from amino to carboxy, a first single domain antibody (1), a linker (2) and a second single domain antibody (3), an IgG hinge sequence (12) and a Fc knob subunit (13) and a second polypeptide chain comprising, from amino to carboxy, a first second domain antibody (3), a linker (2) and a first single domain antibody (1), a IgG hinge sequence (12) and a Fc hole subunit (14), wherein the first and second polypeptides are in stable association via the interaction of the knob-into-hole Fc domain.

FIG. 4, Panel A provides alternative schematic representations of configurations of the bivalent binding molecules of the present disclosure where one single domain antibody is attached to each subunit of a knob-into-hole Fc domain comprising two polypeptides, the first polypeptide comprising from amino to carboxy, a first single domain antibody (1), an IgG hinge sequence (12) and a Fc knob subunit (13), the second polypeptide comprising from amino to carboxy, a second single domain antibody (3), an IgG hinge sequence (12) and a Fc hole subunit (13), wherein the first and second single domain antibodies are in stable associate via the interaction of the knob-into-hole Fc domain.

FIG. 4, Panel B provides a schematic representations of a binding molecule the binding domains are single domain antibodies associated via transition metal coordinate covalent complex. As illustrated, the binding molecules comprises two polypeptide subunits: the first subunit comprising a first single domain antibody (1) is attached via a first linker (15) to a first chelating peptide (17) and second subunit comprising a second single domain antibody (3) is attached via a second linker (16) to a second chelating peptide (18), wherein the first chelating peptide (17) and second chelating peptide (18) form a coordinate covalent complex with a single transition metal ion (“M”). The transition metal ion may be in a kinetically labile or kinetically inert oxidation state.

### DETAILED DESCRIPTION OF THE INVENTION

To facilitate the understanding of present disclosure, certain terms and phrases are defined below as well as throughout the specification. The definitions provided herein are non-limiting and should be read in view of the knowledge of one of skill in the art would know.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); μl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” refers to an activity that promotes cell proliferation and replication.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g. an ortholog, an IL2 ortholog, an engineered cell expressing an orthogonal receptor, an engineered cell expressing an orthogonal IL2 receptor, a CAR-T cell expressing an orthogonal IL2 receptor, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, inhalation and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (K_{D}), a ratio of the dissociation rate constant between the molecule and its target (K_{off}) and the association rate constant between the molecule and its target (Kₒₙ).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction) induced by receptor under study, or a partial agonist. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway, or cell.

Antibody: As used herein, the term “antibody” refers collectively to: (a) glycosylated and non-glycosylated immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, and camelids antibodies (e.g., human antibodies). The term “antibody” encompasses antibodies isolatable from natural sources or from animals following immunization with an antigen and as well as engineered antibodies including monoclonal antibodies, bispecific antibodies, trispecific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries.

Binding molecule: As used herein, the term “binding molecule” refers to a bivalent molecule that can bind to the extracellular domain of two cell surface receptors. In some embodiments, a binding molecule specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

CDR: As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat, et al., U.S. Dept. of Health and Human Services publication entitled “Sequences of proteins of immunological interest” (1991) (also referred to herein as “Kabat 1991” or “Kabat”); by Chothia, et al. (1987) J. Mol. Biol. 196:901-917 (also referred to herein as “Chothia”); and MacCallum, et al. (1996) J. Mol. Biol. 262:732-745, where the definitions include overlapping or subsets of amino acid residues when compared against each other. Nevertheless, application of either definition to refer to a CDR of an antibody or grafted antibodies or variants thereof is intended to be within the scope of the term as defined and used herein. The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs2 and 3 in the variable region of an antibody follows Kabat numbering or simply, “Kabat.” The positioning of CDR1 in the variable region of an antibody follows a hybrid of Kabat and Chothia numbering schemes.

Clonotype: As used herein, a clonotype refers to a collection of binding molecules that originate from the same B-cell progenitor cell. The term “clonotype” is used to refer to a collection of antigen binding molecules that belong to the same germline family, have the same CDR3 lengths, and have 70% or greater homology in CDR3 sequence.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Effective Concentration (EC): As used herein, the terms “effective concentration” or its abbreviation “EC” are used interchangeably to refer to the concentration of an agent (e.g., an hIL2 mutein) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect. The EC of a particular effective concentration of a test agent may be abbreviated with respect to the with respect to particular parameter and test system.

Extracellular Domain: As used herein the term “extracellular domain” or its abbreviation “ECD” refers to the portion of a cell surface protein (e.g. a cell surface receptor) which is outside of the plasma membrane of a cell. The term “ECD” may include the extra-cytoplasmic portion of a transmembrane protein or the extra-cytoplasmic portion of a cell surface (or membrane associated protein).

Identity: As used herein, the term “percent (%) sequence identity” or “substantially identical” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned. Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

Intracellular Signaling: As used herein, the terms “intracellular signaling” and “downstream signaling” are used interchangeably to refer to the to the cellular signaling process that is caused by the interaction of the intracellular domains (ICDs) of two or more cell surface receptors that are in proximity of each other. In receptor complexes via the JAK/STAT pathway, the association of the ICDS of the receptor subunits brings the JAK domains of the ICDs into proximate which initiates a phosphorylation cascade in which STAT molecules are phosphorylated and translocate to the nucleus associating with particular nucleic acid sequences resulting in the activation and expression of particular genes in the cell. The binding molecules of the present disclosure provide intracelluar signaling characteristic of the IFNlambdaR1 receptor when activated by its natural cognate IFNlambda. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be affected by the level of downstream signaling caused by the binding molecule, can also be measured.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polypeptide that may be employed to link two protein domains to provide space and/or flexibility between the two protein domains.

Modulate: As used herein, the terms “modulate”, “modulation” and the like refer to the ability of a test agent to affect a response, either positive or negative or directly or indirectly, in a system, including a biological system or biochemical pathway.

Multimerization: As used herein, the term “multimerization” refers to two or more cell surface receptors, or domains or subunits thereof, being brought in close proximity to each other such that the receptors, or domains or subunits thereof, can interact with each other and cause intracellular signaling.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. The terms “immediately N-terminal” or “immediately C-terminal” are used to refers to a position of a first amino acid residue relative to a second amino acid residue where the first and second amino acid residues are covalently bound to provide a contiguous amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the Operably Linked: The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. In some embodiments, the IFNlambdaR1 binding molecule has a reduced Eₘₐₓ compared to the Eₘₐₓ caused by IFNlambda. Eₘₐₓ reflects the maximum response level in a cell type that can be obtained by a ligand (e.g., a binding molecule described herein or the native cytokine (e.g., IFNlambda)). In some embodiments, the IFNlambdaR1 binding molecule described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IFNlambda. In other embodiments, the Eₘₐₓ of the IFNlambdaR1 binding molecule described herein is greater (e.g., at least 1%, 5%, 10%, 15%, 20%, 25%, 30%, 35%, 40%, 45%, or 50% greater) than the Eₘₐₓ of the natural ligand, IFNlambda. In some embodiments, by varying the linker length of the IFNlambdaR1 binding molecule, the Eₘₐₓ of the IFNlambdaR1 binding molecule can be changed. The IFNlambdaR1 binding molecule can cause Eₘₐₓ in the most desired cell types, and a reduced Eₘₐₓ in other cell types.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Proximity: As used herein, the term “proximity” refers to the spatial proximity or physical distance between two cell surface receptors, or domains or subunits thereof, after a binding molecule described herein binds to the two cell surface receptors, or domains or subunits thereof. In some embodiments, after the binding molecule binds to the cell surface receptors, or domains or subunits thereof, the spatial proximity between the cell surface receptors, or domains or subunits thereof, can be, e.g., less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the spatial proximity amounts to less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. In some embodiments, the receptor is a cell surface receptor that comprises an extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a multimeric complex that results in intracellular signaling.

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and may be designated as such using the abbreviation of a lower case “r” (e.g., rhIL2) to denote the method by which the protein was produced. Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors. In contrast, the terms “inhibition”, “down-regulation” and the like refer to the opposite effects.

Single Domain Antibody (sdAb): The term “single-domain antibody” or “sdAbs,” refers to an antibody having a single (only one) monomeric variable antibody domain. A sdAb is able to bind selectively to a specific antigen. A VHH antibody, further defined below, is an example of a sdAb.

Specifically Binds: As used herein the term “specifically binds” refers to the degree of affinity for which a first molecule exhibits with respect to a second molecule. In the context of binding pairs (e.g., ligand/receptor, antibody/antigen) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the antigen (or antigenic determinant (epitope) of a protein, antigen, ligand, or receptor) if the equilibrium dissociation constant (K_{D}) between antibody and the antigen is lesser than about 10⁻⁶ M, alternatively lesser than about 10⁻⁸ M, alternatively lesser than about 10⁻¹⁰ M, alternatively lesser than about 10⁻¹¹ M, lesser than about 10⁻¹² M as determined by, e.g., Scatchard analysis (Munsen, et al. (1980) Analyt. Biochem. 107:220-239). In one embodiment where the ligand is an ILR binding sdAb and the receptor comprises an ILR, the ILR binding sdAb specifically binds if the equilibrium dissociation constant (K_{D}) of the ILR binding sdAb/ILR ECD is lesser than about 10⁻⁵ M, alternatively lesser than about 10⁻⁶ M, alternatively lesser than about 10₋₇ M, alternatively lesser than about 10⁻⁸ M, alternatively lesser than about 10⁻⁹ M, alternatively lesser than about 10⁻¹⁰ M, or alternatively lesser than about 10⁻¹¹ M. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, e.g., Drescher et al., (2009) Methods Mol Biol 493:323-343 with commercially available instrumentation such as the Biacore 8+, Biacore S200, Biacore T200 (GE Healthcare Bio-Sciences, 100 Results Way, Marlborough MA 01752). In some embodiments, the present disclosure provides molecules (e.g., ILR binding sdAbs) that specifically bind to the hILR. As used herein, the binding affinity of an ILR binding molecule for the ILR, the binding affinity may be determined and/or quantified by surface plasmon resonance (“SPR”). In evaluating binding affinity of an ILR binding molecule for the ILR, either member of the binding pair may be immobilized, and the other element of the binding pair be provided in the mobile phase. In some embodiments, the sensor chip on which the protein of interest is to be immobilized is conjugated with a substance to facilitate binding of the protein of interest such as nitrilotriacetic acid (NTA) derivatized surface plasmon resonance sensor chips (e.g., Sensor Chip NTA available from Cytiva Global Life Science Solutions USA LLC, Marlborough MA as catalog number BR100407), as anti-His tag antibodies (e.g. anti-histidine CM5 chips commercially available from Cytiva, Marlborough MA), protein A or biotin. Consequently, to evaluate binding, it is frequently necessary to modify the protein to provide for binding to the substance conjugated to the surface of the chip. For example, the one member of the binding pair to be evaluated by incorporation of a chelating peptide comprising poly-histidine sequence (e.g., 6xHis (SEQ ID NO: 260) or 8xHis (SEQ ID NO: 261)) for retention on a chip conjugated with NTA. In some embodiments, the ILR binding molecule may be immobilized on the chip and ILR (or ECD fragment thereof) be provided in the mobile phase. Alternatively, the ILR (or ECD fragment thereof) may be immobilized on the chip and the ILR binding molecule be provided in the mobile phase. In either circumstance, it should be noted that modifications of some proteins for immobilization on a coated SPR chip may interfere with the binding properties of one or both components of the binding pair to be evaluated by SPR. In such cases, it may be necessary to switch the mobile and bound elements of the binding pair or use a chip with a binding agent that facilitates non-interfering conjugation of the protein to be evaluated. Alternatively, when evaluating the binding affinity of ILR binding molecule for ILR using SPR, the ILR binding molecule may be derivatized by the C-terminal addition of a poly-His sequence (e.g., 6xHis (SEQ ID NO: 260) or 8xHis (SEQ ID NO: 261)) and immobilized on the NTA derivatized sensor chip and the ILR receptor subunit for which the ILR VHH's binding affinity is being evaluated is provided in the mobile phase. The means for incorporation of a poly-His sequence into the C-terminus of the ILR binding molecule produced by recombinant DNA technology is well known to those of skill in the relevant art of biotechnology. In some embodiments, the binding affinity of ILR binding molecule for an ILR comprises using SPR substantially in accordance with the teaching of the Examples.

Stably Associated: As used herein, the term “stably associated” or “in stable association with” are used to refer to the various means by which one molecule (e.g., a polypeptide) may be associated with another molecule over an extended period of time. The stable association of one molecule to another may be effected by a variety of means, including covalent bonding and non-covalent interactions. In some embodiments, stable association of two molecules may be effected by covalent bonds such as peptide bonds. In other embodiments, stable association of two molecules may be effected b non-covalent interactions. Examples of non-covalent interactions which may providea a stable association between two molecules include electrostatic interactions (e.g., hydrogen bonding, ionic bonding, halogen binding, dipole-dipole interactions, Van der Waals forces and π-effects including cation-n interactions, anion-n interactions and π-π interactions) and hydrophobilic/hydrophilic interactions. In some embodiments, the stable association of sdAbs of the bivalent binding molecules of the present disclosure may be effected by non-covalent interactions. In one embodiment, the non-covalent stable association of the sdAbs of the bivalent binding molecules may be achieved by conjugation of the sdAbs to “knob-into-hole” modified Fc monomers. An Fc “knob” monomer stably associates non-covalently with an Fc “hole” monomer. Conjugation of a first sdAb which specifically binds to the extracellular domain of a first subunit of a heterodimeric receptor to an “Fc knob” monomer and conjugation of an second sdAb which specifically binds to the extracellular domain of a second subunit of a heterodimeric receptor to an “Fc hole” monomer provides stable association of the first and second sdAbs.

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially: As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g., blood count), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

Therapeutically Effective Amount: As used herein, the term The phrase “therapeutically effective amount” is used in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, modification of biomarker levels, increase in duration of survival, extended duration of progression free survival, extension of the time to progression, increased time to treatment failure, extended duration of event free survival, extension of time to next treatment, improvement objective response rate, improvement in the duration of response, and the like that that are relied upon by clinicians in the field for the assessment of an improvement in the condition of the subject in response to administration of an agent.

Treat: The terms “treat”, “treating”, treatment” and the like refer to a course of action (such as administering a binding molecule described herein, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

VHH: As used herein, the term “V_{H}H” is a type of sdAb that has a single monomeric heavy chain variable antibody domain. Such antibodies can be found in or produced from Camelid mammals (e.g., camels, llamas) which are naturally devoid of light chainsV_{H}Hs can be obtained from immunization of camelids (including camels, llamas, and alpacas (see, e.g., Hamers-Casterman, et al. (1993) Nature 363:446-448) or by screening libraries (e.g., phage libraries) constructed in V_{H}H frameworks. Antibodies having a given specificity may also be derived from non-mammalian sources such as V_{H}Hs obtained from immunization of cartilaginous fishes including, but not limited to, sharks. In a particular embodiment, a VHH in a bispecific V_{H}H² binding molecule described herein binds to a receptor (e.g., the first receptor or the second receptor of the natural or non-natural receptor pairs) if the equilibrium dissociation constant between the VHH and the receptor is less than about 10⁻⁶ M, alternatively less than about 10⁻⁸ M, alternatively less than about 10⁻¹⁰ M, alternatively less than about 10⁻¹¹ M, alternatively less than about 10⁻¹⁰ M, or less than about 10⁻¹² M as determined by, e.g., Scatchard analysis (Munsen, et al. 1980 Analyt. Biochem. 107:220-239). Standardized protocols for the generation of single domain antibodies from camelids are well known in the scientific literature. See, e.g., Vincke, et al (2012) Chapter 8 in Methods in Molecular Biology, Walker, J. editor (Humana Press, Totowa NJ). Specific binding may be assessed using techniques known in the art including but not limited to competition ELISA, BIACORE® assays and/or KINEXA® assays. In some embodiments, a V_{H}H described herein can be humanized to contain human framework regions. Examples of human germlines that could be used to create humanized V_{H}Hs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

V_{H}H²: As used herein, the term “V_{H}H²” and “bispecific V_{H}H²” and “VHH dimer” refers to are used interchangeably to refer to a subtype of the binding molecules of the present disclosure wherein the first and second sdAbs are both VHHs and first V_{H}H binding to a first receptor, or domain or subunit thereof, and a second VHH binding to a second receptor, or domain or subunit thereof.

Wild Type: As used herein, the term “wild type” or “WT” or “native” is used to refer to an amino acid sequence or a nucleotide sequence that is found in nature and that has not been altered by the hand of man.

Cytokine Receptor Binding Molecules

General Description

The present disclosure provides disclosure provides cytokine receptor binding molecules that are ligands for a cytokine receptor, the cytokine receptor binding molecule comprising:
(a) a first single domain antibody (sdAb) that specifically binds to the extracellular domain a first subunit of a cytokine receptor; and
(b) a second single domain antibody that specifically binds to extracellular domain of a second subunit of cytokine receptor subunit;
wherein:
the first sdAb and second sdAb are in stable association;
the first and second subunits of the cytokine receptor are dimerized in response to contact with the cognate ligand for the cytokine receptor; and
contacting a cell expressing the first and the second subunits of the cytokine receptor with an effective amount of the cytokine receptor binding molecule results in the intracellular domains of the first and second subunits of the cytokine receptors being brought into proximity and results in intracelluar signaling.

### Single Domain Antibody

The cytokine receptor binding molecules of the present disclosure comprise two or more single domain antibodies. The term “single domain antibody” (sdAb) as used herein refers an antibody fragment consisting of a monomeric variable antibody domain that is able to bind specifically to an antigen and compete for binding with the parent antibody from which it is derived. The term “single domain antibody” includes scFv and VHH molecules. In some embodiments, one or both of the sdAbs of the cytokine receptor binding molecule is a an scFv. In some embodiments, one or both of the sdAbs is a VHH. In some embodiments, one or both of the sdAbs is a scFv.

### Single Domain Antibody Is A VHH

In some embodiments, one or more of the sdAb of the cytokine receptor binding molecules of the present disclosure is a VHH. As used herein, the term “VHH” refers to a single domain antibody derived from camelid antibody typically obtained from immunization of camelids (including camels, llamas and alpacas (see, e.g., Hamers-Casterman, et al. (1993) Nature 363:446-448). VHHs are also referred to as heavy chain antibodies or Nanobodies® as Single domain antibodies may also be derived from non-mammalian sources such as VHHs obtained from IgNAR antibodies immunization of cartilaginous fishes including, but not limited to, sharks. A VHH is a type of single-domain antibody (sdAb) containing a single monomeric variable antibody domain. Like a full-length antibody, it is able to bind selectively to a specific antigen. The complementary determining regions (CDRs) of VHHs are within a single-domain polypeptide. VHHs can be engineered from heavy-chain antibodies found in camelids. An exemplary VHH has a molecular weight of approximately 12-15 kDa which is much smaller than traditional mammalian antibodies (150-160 kDa) composed of two heavy chains and two light chains. VHHs can be found in or produced from Camelidae mammals (e.g., camels, llamas, dromedary, alpaca, and guanaco) which are naturally devoid of light chains. Descriptions of sdAbs and VHHS can be found in, e.g., De Greve et al., Curr Opin Biotechnol. 61:96-101, 2019; Ciccarese, et al., Front Genet. 10:997, 2019; Chanier and Chames, Antibodies (Basel) 8(1), 2019; and De Vlieger et al., Antibodies (Basel) 8(1), 2018.

Engineered sdAbs.

The term single domain antibody includes engineered sdAbs including but not limited to chimeric sdAbs, CDR grafted sdAbs and humanized sdAbs.

In some embodiments, the one or more of the sdAbs for incorporation into the bivalent binding molecules of the present disclosure are CDR grafted. CDRs obtained from antibodies, heavy chain antibodies, and sdAbs derived therefrom may be grafted onto alternative frameworks as described in Saerens, et al. (2005) J. Mol Biol 352:597-607 to generate CDR-grafted sdAbs. Any framework region can be used with the CDRs as described herein.

In some embodiments, one or more of the sdAbs for incorporation into the bivalent binding molecules is a chimeric sdAb, in which the CDRs are derived from one species (e.g., camel) and the framework and/or constant regions are derived from another species (e.g., human or mouse). In specific embodiments, the framework regions are human or humanized sequences. Thus, bivalent binding molecules comprising one or more humanized sdAbs are considered within the scope of the present disclosure. The techniques for humanization of camelid single domain antibodies are well known in the art. See, e.g., Vincke, et al. (2009) General Strategy to Humanize a Camelid Single-domain Antibody and Identification of a Universal Humanized Nanobody Scaffold J. Biol. Chem. 284(5)3273-3284.

In some embodiments, a VHH described herein can be humanized to contain human framework regions. Examples of human germlines that could be used to create humanized VHHs include, but are not limited to, VH3-23 (e.g., UniProt ID: P01764), VH3-74 (e.g., UniProt ID: A0A0B4J1X5), VH3-66 (e.g., UniProt ID: AOAOC4DH42), VH3-30 (e.g., UniProt ID: P01768), VH3-11 (e.g., UniProt ID: P01762), and VH3-9 (e.g., UniProt ID: P01782). */*

Stably Associated

The present disclosure provides a synthetic cytokine receptor ligand comprising at least two binding domains, the synthetic ligand comprising a first binding domain that specifically binds to the extracellular domain of a first cytokine receptor subunit in stable association with a second binding domain that specifically binds to the extracellular domain of a second cytokine receptor subunit. As used herein, the term “stably associated” or “in stable association with” are used to refer to the various means by which one molecule (e.g., a polypeptide) may be thermodynamically and/or kinetically associated with another molecule. The stable association of one molecule to another may be achieved by a variety of means, including covalent bonding and non-covalent interactions.

Covalent Bonding

In some embodiments, stable association of two molecules may be effected by covalent bonds such as peptide bonds. In some embodiments, the covalent linkage between the first and second binding domains is a covalent bond between the C-terminus of the first binding domain and the N-terminus of the second binding domain.

In some embodiments, the first binding domain that specifically binds to the extracellular domain of a first cytokine receptor subunit in stable association with a second binding domain that specifically binds to the extracellular domain of a second cytokine receptor subunit are covalent bonded via a linker. In some embodiments, a linker joins the C-terminus of the first sdAb which binds to the ECD of the first receptor subunit of the cytokine receptor of the binding molecule to the N-terminus of the second sdAb which binds to the ECD of the second receptor subunit of the cytokine receptor. In some embodiments, a linker joins the C-terminus of the second sdAb which binds to the ECD of the second receptor subunit of the cytokine receptor of the binding molecule to the N-terminus of the first sdAb which binds to the ECD of the first receptor subunit of the cytokine receptor. Linkers may be selected from selected from the group including but not limited to peptide linkers or chemical linkers.

Peptide Linkers

In some embodiments, the stable association of the first and second domains may be achieved by covalent linkage of the C-terminus of the first binding domain and the N-terminus of the second binding domain via a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). Examples of flexible peptide linkers include glycine polymers (G)n, glycine-alanine polymers, alanine-serine polymers, glycine-serine polymers (for example, (GmSo)n (SEQ ID NO: 262), (GSGGS)n (SEQ ID NO: 263), (GmSoGm)n (SEQ ID NO: 264), (GmSoGmSoGm)n (SEQ ID NO: 265), (GSGGSm)n (SEQ ID NO: 266), (GSGSmG)n (SEQ ID NO: 267) and (GGGSm)n (SEQ ID NO: 268), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Exemplary flexible linkers include the linkers of but are not limited to GGGS (SEQ ID NO:13), GGGGS (SEQ ID NO: 14), GGSG (SEQ ID NO: 15), GGSGG (SEQ ID NO: 16), GSGSG (SEQ ID NO: 17), GSGGG (SEQ ID NO: 18), GGGSG (SEQ ID NO: 19) and GSSSG (SEQ ID NO: 20). In yet other embodiments, a peptide linker can contain 4 to 20 amino acids including mixtures of the above motifs of GGSG (SEQ ID NO:15), e.g., GGSGGGSG (SEQ ID NO:21), GGSGGGSGGGSG (SEQ ID NO:22), GGSGGGSGGGSGGGSG (SEQ ID NO:23), or GGSGGGSGGGSGGGSGGGSG (SEQ ID NO:24). In other embodiments, a peptide linker can contain motifs of GGSG (SEQ ID NO:15), e.g., GGSGGGSG (SEQ ID NO:21), GGSGGGSGGGSG (SEQ ID NO:22), GGSGGGSGGGSGGGSG (SEQ ID NO:23), or GGSGGGSGGGSGGGSGGGSG (SEQ ID NO:24).

Chemical Linkers

In some embodiments, the stable association of the first and second domains may be achieved by a chemical linkage. Examples of chemical linkers include aryl acetylene, ethylene glycol oligomers containing 2-10 monomer units, diamines, diacids, amino acids, or combinations thereof. In some embodiments, the linker is a peptide linker. Suitable peptide linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Suitable peptide linkers are known in the art, and include, for example, peptide linkers containing flexible amino acid residues such as glycine and serine. Examples of flexible linkers include glycine polymers (G)ₙ, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)ₙ, glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of such linker sequences may be linked together to provide flexible linkers that may be used to conjugate a heterologous amino acid sequence to IFNlambdaR1 binding sdAbs disclosed herein. In some embodiments the linkers have the formula (GGGS)n (SEQ ID NO: 269), (GGGSG)n (SEQ ID NO: 270), (GGS)nG (SEQ ID NO: 271), or (GGSG)n (SEQ ID NO: 272), wherein n is an integer selected from 1, 2, 3, 4, 5, 6, 7, 8, 9 and 10.

Non-Covalent Bonding

In some embodiments, stable association of the first and second binding domains of the binding molecules may be effected by non-covalent interactions. Examples of non-covalent interactions which may providea a stable association between two molecules include electrostatic interactions (e.g., hydrogen bonding, ionic bonding, halogen binding, dipole-dipole interactions, Van der Waals forces and p-effects including cation-p interactions, anion-p interactions and p-p interactions) and hydrophobilic/hydrophilic interactions. In some embodiments, the stable association of sdAbs of the binding molecules of the present disclosure may be effected by non-covalent interactions. In one embodiment, the non-covalent stable association of a receptor binding molecules to a subunit of an Fc, domain optionally incorporating a linker between the receptor binding molecule and the Fc such as the IgG4 hinge comain. Alternatively, the receptor binding molecule or individual sdAbs of the binding molecules may be achieved by conjugation to a domain (or both domains) of the sdAbs to “knob-into-hole” modified Fc monomers.

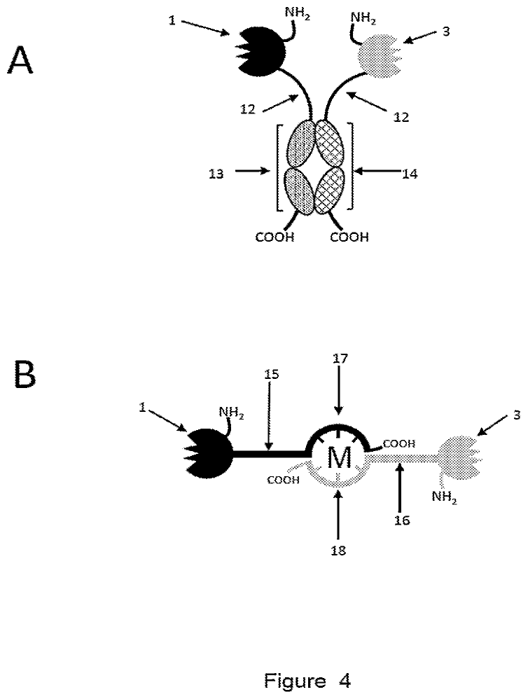

In one embodiment, the non-covalent stable association of the sdAbs of the binding molecules may be achieved by conjugation of the sdAbs to “knob-into-hole” modified Fc monomers. An Fc “knob” monomer stably associates non-covalently with an Fc “hole” monomer. Conjugation of a first sdAb which specifically binds to the extracellular domain of a first subunit of a heterodimeric receptor to an “Fc knob” monomer and conjugation of an second sdAb which specifically binds to the extracellular domain of a second subunit of a heterodimeric receptor to an “Fc hole” monomer provides stable association of the first and second sdAbs. The knob-into-hole modification is more fully described in Ridgway, et al. (1996) Protein Engineering 9(7):617-621 and U.S. Pat. No. 5,731,168, issued Mar. 24, 1998, U.S. Pat. No. 7,642,228, issued Jan. 5, 2010, U.S. Pat. No. 7,695,936, issued Apr. 13, 2010, and U.S. Pat. No. 8,216,805, issued Jul. 10, 2012. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”) and ii) in the CH3 domain of a second heavy chain, an amino acid residue is replaced with an amino acid residue having a smaller side chain (e.g., alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 on one chain and Y349 on the second chain which results in a stabilizing disulfide bridge between the two antibody heavy chains in the Fce region (Carter, et al. (2001) Immunol Methods 248, 7-15). The knob-into-hole format is used to facilitate the expression of a first polypeptide (e.g., an IL27Ra binding sdAb) on a first Fc monomer with a “knob” modification and a second polypeptide on the second Fc monomer possessing a “hole” modification to facilitate the expression of heterodimeric polypeptide conjugates. A schematic illustration of this wherein each binding domain is be provided on separate subunits of a knob-into-hole Fc dimer such that the first and second binding domains are non-covalently linked via the non-covalent linkage of the knob and hole as illustrated in FIG. 4, Panel A of the attached drawings.

### Coordinate Covalent Bonding

In some embodiments, stable association of the first and second binding domains of the binding molecules may be effected by a coordinate covalent linkage. The present disclosure provides examples of single domain antibodies comprising a chelating peptide. The chelating peptide results in a coordinate covalent linkage to a transition metal ion. In some embodiments, a transition metal ion is capable of forming a coordinate covalent linkage with two or more chelating peptides. Consequently, the first and second binding domains may each comprise a chelating peptide and a stable association of the binding domains by each subunit forming a coordinate covalent complex with a transition metal ion. In some embodiments, the transition metal ion is selected from vanadium, manganese, iron, iridium, osmium, rhenium platinum, palladium, cobalt, chromium or ruthenium. A schematic illustration of this configuration is provided in FIG. 4, Panel B of the attached drawings. It should be noted that in each of the configurations illustrated in FIG. 4, Panels A and B, the N-terminal domain of the single domain antibody is presented to the environment enabling facilitating enhanced exposure of the CDRs of the sdAb to the target cytokine receptor ECD. The formation of the coordinate covalent linkage between the is favored when the transition metal ion is in a kinetically labile oxidation state, for example Co(II), Cr(II), or Ru(III). Following complexation, the oxidation state of the transition metal may be changed (oxidized or reduced) to a kinetically inert oxidation state, for example Co(III), Cr(III), or Ru(II), provide a kinetically inert coordinate covalent complex. The the formation of kinetically inert and kinetically labile coordinate covalent complexes between proteins comprising chelating peptides via a transition metal are described in more detail in Anderon, et al. U.S. Pat. No. 5,439,928 issued Aug. 8, 1995.

### Modulation of Activity of Receptor Binding Molecules

In some embodiments, such as to achieve partial agonism or selective activation of particular cell types, the design of the cytokine receptor binding molecules of the present disclosure may be modulated by structural variations in the design of the receptor binding molecule. This variation in activity may be employed to modulated the binding and activity of the receptor binding molecule, for example to variations in chieve partial agonism, selective cell type activation or increased or decreased activity relative to the cognate ligand for the receptor. Examples of the means by which the modulation of the activity and/or specificity of the receptor binding molecule of the present disclosure include but are not limited to altering the sequential orientation of the sdAb, independently varying the of the binding affinity of the sdAbs with respect to each target, and modulating the distance between the sdAbs such as by employing linkers or varying lengths.

In some embodiments, the cytokine receptor binding protein has a reduced Eₘₐₓ compared to the Eₘₐₓ caused by the cognate ligand. Eₘₐₓ reflects the maximum response level in a cell type that can be obtained by a ligand (e.g., a binding protein described herein or the cognate ligand (e.g., IFNLambda1)). In some embodiments, the IFNLambdaR1 binding protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by hIFNLambda1.

Modulation of Activity by Varying the Distance Between the sdAbs

In some embodiments, by modulating the distance between the sdAbs of receptor binding protein (e.g. by varying the linker length between the sdAbs), the Eₘₐₓ of the binding protein can be modulated. The such variations in receptor binding protein geometry can exploited to increase activity in the most desired cell types (e.g., CD8⁺ T cells), while reducing activity in other cell types (e.g., macrophages). With respect to the IFNlambdaR1 binding molecules, in some embodiments, the Eₘₐₓ of the IFNlambdaR1 binding protein on macrophages is between 1% and 100% (e.g., between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ the IFNlambdaR1 binding protein on T cells (e.g., CD8⁺ T cells). In other embodiments, the Eₘₐₓ of the IFNlambdaR1 binding protein described herein is greater (e.g., at least 1%, 5%, 10%, 15%, 20%, 25%, 30%, 35%, 40%, 45%, or 50% greater) than the Eₘₐₓ of the natural ligand, IFNlambda1.

Sequential Orientation of sdAbs

When the cytokine receptor binding molecule of the present disclosure is expressed as a single polypeptide chain, the binding activity and/or specificity of the receptor binding molecule may be modulated by the order (N-terminal versus C-terminal) arraignments of the sdAbs in the polypeptide. In some embodiments, the cytokine receptor binding molecule is a polypeptide IFNlambdaR1 binding molecule and the activity and/or specificity of the IFNlambdaR1 binding is modulated by the sequential arrangement of the IFNlambdaR1 and IL28RA sdAbs in the polypeptide.

“Forward Orientation”

In some embodiments, the cytokine receptor binding molecule (e.g., an IFNlambdaR1 binding molecule) comprises a polypeptide of the structure:

H₂N-[First Receptor Subunit sdAb]-[L]ₓ-[Second Receptor Subunit sdAb]-[CP]_{y}—COOH wherein and L is a polypeptide linker of 1-50 amino acids and x=0 or 1, and CP is a chelating peptide or a subunit of an Fc domain and y=0 or 1.

In one embodiment, the present disclosure provides a IFNlambdaR1 binding molecule comprises a polypeptide of the structure:

H₂N-[IL10Rb sdAb]-[L]x-[IL28RA sdAb]-[CP]_{y}—COOH wherein and L is a polypeptide linker of 1-50 amino acids and x=0 or 1, and CP is a chelating peptide or a subunit of an Fc domain and y=0 or 1. This is referred to herein as the “forward orientation” of the IL10Rb and IL28RA sdAbs of the IFNlambdaR1 binding molecule.

“Reverse Orientation”

In some embodiments, the cytokine receptor binding molecule (e.g., an IFNlambdaR1 binding molecule) comprises a polypeptide of the structure:

H₂N-[Second Receptor Subunit sdAb]-[L]ₓ-[First Receptor Subunit sdAb]-[CP]_{y}—COOH

wherein L is a polypeptide linker of 1-50 amino acids and x=0 or 1, and CP is a chelating peptide or a subunit of an Fc domain and y=0 or 1.

In some embodiments, the bivalent IFNlambdaR1 binding molecule comprises a polypeptide of the structure:

H₂N-[IL28RA sdAb]-[L]ₓ-[IL10Rb sdAb]-[CP]_{y}—COOH

wherein L is a polypeptide linker of 1-50 amino acids and x=0 or 1, and CP is a chelating peptide or a subunit of an Fc domain, and y=0 or 1.
Modulation of Activity Variation of The Binding Affinities of sdAbs

In some embodiments, the activity and/or specificity of the bivalent receptor binding molecule of the present disclosure may be modulated by independently varying the respective binding affinities of the first and second sdAbs for their respective receptor subunits.

It will be appreciated by one of skill in the art that the binding of the first sdAb of the bivalent binding molecule to the first receptor subunit ECD on the cell surface will enhance the probability of a binding interaction between the second sdAb of the bivalent binding molecule with the ECD of the second receptor subunit. This cooperative binding effect may result in a bivalent receptor binding molecule which has a very high affinity for the receptor and a very slow “off rate” from the receptor. Typical VHH single domain antibodies have an affinity for their targets of from about 10⁻⁵ M to about 10⁻¹⁰ M. In those instances such slow off-rate kinetics are desirable in the bivalent IFNlambdaR1 binding molecules, the selection of sdAbs having high affinities (about 10⁻⁷ M to about 10⁻¹⁰ M) for incorporation into the bivalent IFNlambdaR1 binding molecule are favored.

Naturally occurring cytokine ligands for typically do not exhibit a similar affinity for each subunit of a heterodimeric receptor. Consequently, in designing a bivalent cytokine receptor binding molecule which is a mimetic of the cognate cytokine ligand as contemplated by some embodiments of the present disclosure, selection of sdAbs for the first and second receptor receptor subunit have an affinity similar to (e.g., having an affinity about 10 fold, alternatively about 20 fold, or alternatively about 50 fold higher or lower than) the cognate IFNlambda1 for the respective receptor subunit may be used.

In some embodiments, the bivalent receptor binding molecules of the present disclosure are partial agonists. As such, the activity of the binding molecule may be modulated by selecting sdAb which have greater or lesser affinity for either one or both of the receptor receptor subunits relative to the cognate ligand. As some heterodimeric cytokine receptors are comprised of a “proprietary subunit” (i.e., a subunit which is not naturally a subunit of another multimeric receptor) and a second “common” subunit (such as IL28RA) which is a shared component of multiple cytokine receptors), selectivity for the formation of such receptor may be enhanced by employing first sdAb which has a higher affinity for the proprietary receptor subunit and second sdAb which exhibits a lower affinity for the common receptor subunit. Additionally, the common receptor subunit may be expressed on a wider variety of cell types than the proprietary receptor subunit. In some embodiments wherein the receptor is a heterodimeric receptor comprising a proprietary subunit and a common subunit, the first sdAb of the bivalent IFNlambdaR1 binding molecule exhibits a significantly greater (more than 10 times greater, alternatively more than 100 times greater, alternatively more than 1000 times greater) affinity for the proprietary receptor than the second sdAb of the bivalent IFNlambdaR1 binding molecule for the common receptor subunit. In one embodiment, the present disclosure provides a bivalent IFNlambdaR1 binding molecule wherein the affinity of the IL10Rb sdAb has an affinity of more than 10 times greater, alternatively more than 100 times greater, alternatively more than 1000 times greater than the affinity for the IL28RA sdAb common receptor subunit.

With respect to the IFNlambdaR1 binding molecules of the present disclosure, in some embodiments, the affinity of the IL10Rb sdAb of the IFNlambdaR1 binding molecule for the IFNlambdaR1 ECD is at least about 2 fold, alternatively at least about 5 fold, alternatively at least about 5 fold, alternatively at least about 10 fold, alternatively at least about 50 fold, alternatively at least about 100 fold, alternatively at least about 200 fold, alternatively at least about 500 fold, or alternatively at least about 1000 fold greater than the binding affinity of IL28RA sdAb of the IFNlambdaR1 binding molecule for the IL28RA ECD. In some embodiments, the affinity of the IL28RA sdAb of the IFNlambdaR1 binding molecule for the IL28RA ECD is at least about 2 fold, alternatively at least about 5 fold, alternatively at least about 5 fold, alternatively at least about 10 fold, alternatively at least about 50 fold, alternatively at least about 100 fold, alternatively at least about 200 fold, alternatively at least about 500 fold, or alternatively at least about 1000 fold greater than the binding affinity of IL10Rb sdAb of the IFNlambdaR1 binding molecule for the IFNlambdaR1 ECD.

Cross Reactivity:

In some instances, due to sequence or structural similarities between the extracellular domains of IL10Rb receptors from various mammalian species, immunization with an antigen derived from a IL10Rb of a first mammalian species (e.g., the hIL10Rb-ECD) may provide antibodies which specifically bind to IL10Rb receptors of one or more additional mammalian species. Such antibodies are termed “cross reactive.” For example, immunization of a camelid with a human derived antigen (e.g., the hIL10Rb-ECD) may generate antibodies that are cross-reactive the murine and human receptors. Evaluation of cross-reactivity of antibody with respect to the receptors derived from other mammalian species may be readily determined by the skilled artisan, for example using the methods relating to evaluation of binding affinity and/or specific binding described elsewhere herein such as flow cytometry or SPR. Consequently, the use of the term “human IL10Rb VHH” or “hIL10Rb VHH” merely denotes that the species of the IL10Rb antigen used for immunization of the camelid from which the VHH was derived was the human IL10Rb (e.g., the hIL10Rb ECD, SEQ ID NO:2) but should not be understood as limiting with respect to the specific binding affinity of the VHH for IL10Rb molecules of other mammalian species. Similarly, the use of the term “mouse IL10Rb VHH” or “mIL10Rb VHH” merely denotes that the species of the IL10Rb antigen used for immunization of the camelid from which the VHH was derived was the murine IL10Rb (e.g., the mIL10Rb ECD, SEQ ID NO: 4) but should not be understood as limiting with respect to the specific binding affinity of the VHH for IL10Rb molecules of other mammalian species.

The hIL10Rb VHHs of Table 5 were evaluated for cross-reactivity with the mIL10Rb by flow cytometry and were found to bind both the extracellular domain of hIL10Rb (SEQ ID NO.2) and the extracellular domain of mIL10Rb (SEQ ID NO.4). Consequently, the VHHs provided in Table 5 may be used in both murine and human applications avoiding the necessity of a surrogate anti-mIL10Rb for anti-hIL10Rb for in vivo models of efficacy, such as a mouse model of a human disease state.

In some instances, due to sequence or structural similarities between the extracellular domains of IL10Rb receptors from various mammalian species, immunization with an antigen derived from a IL10Rb of a first mammalian species (e.g., the hIL10Rb-ECD) may provide antibodies which specifically bind to IL10Rb receptors of one or more additional mammalian species. Such antibodies are termed “cross reactive.” For example, immunization of a camelid with a human derived antigen (e.g., the hIL10Rb-ECD) may generate antibodies that are cross-reactive the murine and human receptors. Evaluation of cross-reactivity of antibody with respect to the receptors derived from other mammalian species may be readily determined by the skilled artisan, for example using the methods relating to evaluation of binding affinity and/or specific binding described elsewhere herein such as flow cytometry or SPR. Consequently, the use of the term “human IL10Rb VHH” or “hIL10Rb VHH” merely denotes that the species of the IL10Rb antigen used for immunization of the camelid from which the VHH was derived was the human IL10Rb (e.g., the hIL10Rb ECD, SEQ ID NO:2) but should not be understood as limiting with respect to the specific binding affinity of the VHH for IL10Rb molecules of other mammalian species. Similarly, the use of the term “mouse IL10Rb VHH” or “mIL10Rb VHH” merely denotes that the species of the IL10Rb antigen used for immunization of the camelid from which the VHH was derived was the murine IL10Rb (e.g., the mIL10Rb ECD, SEQ ID NO:4) but should not be understood as limiting with respect to the specific binding affinity of the VHH for IL10Rb molecules of other mammalian species.

I. Interferon Lambda Receptor 1 Binding Molecules

In one embodiment, the present disclosure provides an IFNlambdaR1 binding molecule that is a ligand for the IFNlambdaR1, the IFNlambdaR1 binding molecule comprising:
a first single domain antibody (sdAb) that specifically binds to the extracellular domain of IL10Rb subunit of the IFNlambdaR1 (an “anti-IL10Rb sdAb”), and a second single domain antibody that specifically binds to extracellular domain of
IL28RA subunit of the IFNlambdaR1 (an “anti-IL28RA sdAb”),
wherein:
the first sdAb and second sdAb are in stable association;
the IL10Rb and IL28RA subunits of the IFNlambdaR1 are dimerized in response to contact with the IFNlambdaR1 binding molecule; and
contacting a cell expressing the IL10Rb and IL28RA with an effective amount of the IFNlambdaR1 binding molecule results in the intracellular domains of IL10Rb and IL28RA being brought into proximity and intracellular signaling.

In some embodiments, one or both of the sdAbs is a an scFv. In some embodiments, one or both of the sdAbs is a VHH.

As used herein, the term “IFNlambdaR1 receptor” or “IFNlambdaR1” refers to a heterodimeric receptor formed by subunits IL10Rb and IL28RA when associated with the cognate IFNlambda1.

The amino acid sequence of the mature form (less the signal peptide) of human IL28RA is provided as SEQ ID NO:______. The human sequence of IL28RA is listed as UniProt ID NO. P38484.

The IFNlambda1 receptor (IFNlambdaR1) includes IL10Rb subunit (IL10Rb) and IL28RA subunit (IL28RA). Provided herein is an IFNlambdaR1 binding molecule that specifically binds to IL10Rb and IL28RA. In some embodiments, the IFNlambdaR1 binding molecule binds to a mammalian cell expressing both IL10Rb and IL28RA. In some embodiments, the IFNlambdaR1 binding molecule can be a bispecific VHH 2 as described below.

IFNlambda1: the Cognate Ligand for the IFNlambda1 Receptor

The cognate ligand for the IFNlambda1 receptor is the cytokine IFNlambda1. IFNlambda1 is a homodimeric polypeptide which is an agonist of the IFNlambdaR1. Human IFNlambda1 is a non-covalently linked homodimeric protein comprising two identical subunits. The canonical amino acid sequence of one subunit of the homodimeric human IFNlambda1 is provided below (UniProt Reference No: Q8IU54).

(SEQ ID NO: 9)
MAAAWTVVLVTLVLGLAVAGPVPTSKPTTTGKGCHIGRFKSLSPQELAS
FKKARDALEESLKLKNWSCSSPVFPGNWDLRLLQVRERPVALEAELALT
LKVLEAAAGPALEDVLDQPLHTLHHILSQLQACIQPQPTAGPRPRGRLH
HWLHRLQEAPKKESAGCLEASVTFNLFRLLTRDLKYVADGNLCLRTSTH
PEST

IFNlambda Receptor1 (IFNlambdaR1)

The present disclosure relates to synthetic mimetics of the naturally occurring IFNlambda1 which are agonists of the IFNlambdaR1. The IFNlambdaR1 is a heterodimeric protein complex of IL10Rb and IL28RA. The binding of the IFNlambda1 results in dimerization IL10Rb and IL28RA and intracellular signaling in cells expressing IL10Rb and IL28RA characteristic of the binding of the naturally occurring IFNlambda1 for the IFNlambdaR1. In some embodiments, the IFNlambdaR1 is the human IFNlambdaR1 and the IFNlambda1 is the human IFNlambda1. In some embodiments the IFNlambdaR1 is the murine IFNlambdaR1 and the IFNlambda1 is the murine IFNlambda1. As used herein, the terms “IFNlambda1 receptor” and “IFNlambdaR1” are used interchangeably to refer to a heterodimeric complex comprising IL10Rb and IL28RA. The term IFNlambdaR1 includes IFNlambda1 receptors of any mammal including but not limited to human beings, dogs, cats, mice, monkeys, cows, and pigs.

IFNlambda1 Receptor Subunit IL10Rb

The present disclosure provides sdAbs that specifically bind to the extracellular domain of the IL10Rb and IFNlambdaR1 binding molecules comprising such sdAbs. In some embodiments, the IFNlambdaR1 binding molecules of the present disclosure specifically bind to the extracellular domain of the IFNlambdaR1.

Human IL10Rb:

In one embodiment, the IL10Rb is the human IL10Rb (hIL10Rb). The hIL10Rb is expressed as a 325 amino acid pre-protein, the first 19 amino acids comprising a signal sequence which is post-translationally cleaved in the mature 306 amino acid protein. Amino acids 20-220 (amino acids 1-201 of the mature protein) correspond to the extracellular domain, amino acids 221-242 (amino acids 202-223 of the mature protein) correspond to the 22 amino acid transmembrane domain, and amino acids 243-325 (amino acids 224-306 of the mature protein) correspond to the intracellular domain. hIL10Rb is referenced at UniProtKB database as entry Q08334. The canonical full length hIL10Rb precursor is a polypeptide having the amino acid sequence:

(SEQ ID NO: 1)
MAWSLGSWLGGCLLVSALGMVPPPENVRMNSVNFKNILQWESPAFAKG
NLTFTAQYLSYRIFQDKCMNTTLTECDFSSLSKYGDHTLRVRAEFADEH
SDWVNITFCPVDDTIIGPPGMQVEVLADSLHMRFLAPKIENEYETWTMK
NVYNSWTYNVQYWKNGTDEKFQITPQYDFEVLRNLEPWTTYCVQVRGFL
PDRNKAGEWSEPVCEQTTHDETVPSWMVAVILMASVFMVCLALLGCFAL
LWCVYKKTKYAFSPRNSLPQHLKEFLGHPHHNTLLFFSFPLSDENDVFD
KLSVIAEDSESGKQNPGDSCSLGTPPGQGPQS

To generate sdAbs against the human IL10Rb, the extracellular domain of the hIL10Rb protein was used as an immunogen. The extracellular domain of the mature (lacking the signal sequence) hIL10Rb possesses the amino acid sequence:

(SEQ ID NO: 2)
MVPPPENVRMNSVNFKNILQWESPAFAKGNLTFTAQYLSYRIFQDKCMN
TTLTECDFSSLSKYGDHTLRVRAEFADEHSDWVNITFCPVDDTIIGPPGM
QVEVLADSLHMRFLAPKIENEYETWTMKNVYNSWTYNVQYWKNGTDE
KFQITPQYDFEVLRNLEPWTTYCVQVRGFLPDRNKAGEWSEPVCEQTTH
DETVPS

For purposes of the present disclosure, the numbering of amino acid residues of the human IL10Rb polypeptides as described herein is made in accordance with the numbering of this canonical sequence (UniProt ID: Q08334). Amino acids 1-19 of SEQ ID NO:1 are identified as the signal peptide of the IL10Rb, amino acids 20-220 of SEQ ID NO:1 are identified as the extracellular domain, amino acids 221-242 of SEQ ID NO:1 are identified as the transmembrane domain, and amino acids 243-325 of SEQ ID NO:1 are identified as the intracellular domain.

Murine IL10Rb

In one embodiment, the IL10Rb is the murine IL10Rb. Murine IL10Rb (mIL10Rb) is expressed as a 349 amino acid pre-protein comprising a 19 amino acid N-terminal signal sequence. Amino acids 20-220 (amino acids 1-201 of the mature protein) correspond to the extracellular domain, amino acids 221-241 (amino acids 202-222 of the mature protein) correspond to the 21 amino acid transmembrane domain, and amino acids 242-349 (amino acids 223-330 of the mature protein) correspond to the intracellular domain. mIL10Rb is referenced at UniProtKB database as entry Q61190.

The canonical full length mIL10Rb precursor protein including the signal sequence is a polypeptide of the amino acid sequence:

(SEQ ID NO: 3)
MAPCVAGWLGGFLLVPALGIPPPEKVRMNSVNFKNILQWEVPAFPKTNL
TFTAQYESYRSFQDHCKRTASTQCDFSHLSKYGDYTVRVRAELADEHSE
WVNVTFCPVEDTIIGPPEMQIESLAESLHLRFSAPQIENEPETWTLKNI
YDSWAYRVQYWKNGTNEKFQVVSPYDSEVLRNLEPWTTYCIQVQGFLLD
QNRTGEWSEPICERTGNDEITPSWIVAIILIVSVLVVFLFLLGCFVVLW
LIYKKTKHTFRSGTSLPQHLKEFLGHPHHSTFLLFSFPPPEEAEVFDKL
SIISEESEGSKQSPEDNCASEPPSDPGPRELESKDEAPSPPHDDPKLLT
STSEV

To generate sdAbs against mIL10Rb, the extracellular domain of the mIL10Rb protein was used as an immunogen. The extracellular domain of the mature (lacking the signal sequence) mIL10Rb possesses the amino acid sequence (amino acids 27-240):

(SEQ ID NO: 4)
MIPPPEKVRMNSVNFKNILQWEVPAFPKTNLTFTAQYESYRSFQDHCKRT
ASTQCDFSHLSKYGDYTVRVRAELADEHSEWVNVTFCPVEDTIIGPPEMQ
IESLAESLHLRFSAPQIENEPETWTLKNIYDSWAYRVQYWKNGTNEKFQV
VSPYDSEVLRNLEPWTTYCIQVQGFLLDQNRTGEWSEPICERTGNDEITP
S

For purposes of the present disclosure, the numbering of amino acid residues of the murine IL10Rb polypeptides as described herein is made in accordance with the numbering of this canonical sequence (UniProt ID: Q61190. Amino acids 1-19 of SEQ ID NO:168 are identified as the signal peptide of the IL10Rb, amino acids 20-220 of SEQ ID NO:168 are identified as the extracellular domain, amino acids 221-241 of SEQ ID NO:168 are identified as the transmembrane domain, and amino acids 242-349 of SEQ ID NO:168 are identified as the intracellular domain.

### Generation and Evaluation of IL10Rb Single Domain Antibodies

A series of IL10RB sdAbs were generated in substantial accordance with the teaching of Examples 1˜4 herein. Briefly, a camel was immunized with the extracellular domain (amino acids 20-220) of IL10RB (UNIPROT Ref: Q08334). A synthetic DNA sequence encoding the antigen was inserted into the pFUSE_hIgG1_Fc2 vector (Generay Biotechnology) and transfected into the HEK293F mammalian cell host cell for expression. The antigen is expressed as an Fc fusion protein which is purified using Protein A chromatography. A series of VHHs was generated in response to this procedure and are provided in Tables 5 and 6.

Exemplary IL10Rb Single Domain Antibodies

Tables 2 and 3 provide CDRs useful in the preparation of IL10Rb sdAbs for incorporation into the binding molecules of the present disclosure. In some embodiments, the IL10Rb sdAbs are generated in response to immunization with the extracellular domain and specifically bind to the ECD of hIL10Rb. In some embodiments, the IL10Rb sdAb is a single domain antibody comprising: a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR1s in Table 2 or 3; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR2s in Table 2 or 3; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one the CDR3s in Table 2 or 3.

In some embodiments, the IL10Rb sdAb comprises a VHH amino acid sequence having at least 90% (e.g., 91%, 92%, 93%, 94%, 95%, 96%, 97%, 98%, 99%, or 100%) sequence identity to a sequence of any one the of IL10Rb sdAb s provided in Table 5 or 6. In certain embodiments, the binding molecule comprises a sequence that is substantially identical to a sequence of any one of the sequences listed in a row of Table 5 or 6. In certain embodiments, the binding molecule comprises a sequence that is identical to a sequence of any one of the sequences listed in a row of Table 5 or 6.

In another aspect, the disclosure provides an isolated nucleic acid encoding an IL10Rb sdAb described herein. Tables 8 and 9 provide DNA sequences encoding the IL10Rb sdAbs of Table 5 and 6. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence of Table 8 or 9. In certain embodiments, the present disclosure provides an isolated nucleic acid comprising a DNA sequence that is substantially identical to a DNA sequence of Table 8 or 9. In certain embodiments, the present disclosure provides an isolated nucleic acid comprising a DNA sequence that is identical to a DNA sequence of Table 8 or 9.

### IL10Rb Binding Molecules and Single Domain Antibodies

In some embodiments, a IL10Rb binding molecule of the present disclosure is a single domain antibody (sdAb). The present disclosure relates to IL10Rb binding molecules comprising single domain antibodies (sdAbs) that specifically bind to the extracellular domain of the human IL10Rb isoform (hIL10Rb) which are found on all IL10Rb-expressing cells.

A single-domain antibody (sdAb) is an antibody containing a single monomeric variable antibody domain. Like a full-length antibody, sdAbs are able to bind specifically to an antigenic determinant. hIL10Rb binding VHH single-domain antibodies can be engineered from heavy chain antibodies isolated from Camelidae mammals (e.g., camels, llamas, dromedary, alpaca, and guanaco) immunized with the extracellular domain of hIL10Rb or an immunologically active fragment thereof. Descriptions of sdAbs and VHHs can be found in, e.g., De Greve et al., (2019) Curr Opin Biotechnol. 61:96-101; Ciccarese, et al., (2019) Front Genet. 10:997: Chanier and Chames (2019) Antibodies (Basel) 8(1); and De Vlieger, et al. (2018) Antibodies (Basel) 8(1). Alternatively, hIL10Rb single domain antibodies may be engineered from heavy chain antibodies isolated from the IgNAR heavy chain antibodies isolated from cartilaginous fishes immunized with the extracellular domain of hIL10Rb or an immunologically active fragment thereof hIL10Rb binding sdAbs may also be obtained by splitting the dimeric variable domains from immunoglobulin G (IgG) isotypes from other mammalian species including humans, rats, rabbits immunized with the extracellular domain of hIL10Rb or an immunologically active fragment thereof. Although most research into sdAbs is currently based on heavy chain variable domains, sdAbs derived from light chains have also been shown to bind specifically to the target proteins comprising the antigenic immunization sequence. Moller et al., J Biol Chem. 285(49):38348-38361, 2010.

In some embodiments, the sdAb is a VHH. A VHH is a type of sdAb that has a single monomeric heavy chain variable antibody domain. Similar to a traditional antibody, a VHH is able to bind specifically to a specific antigen. An exemplary VHH has a molecular weight of approximately 12-15 kDa which is much smaller than traditional mammalian antibodies (150-160 kDa) composed of two heavy chains and two light chains. VHHs can be found in or produced from Camelidae mammals (e.g., camels, llamas, dromedary, alpaca, and guanaco) which are naturally devoid of light chains.

### Modified Forms of Single Domain Antibodies

CDR Grafted sdAbs

In some embodiments, the IL10Rb binding sdAb of the present disclosure is a CDR grafted IL10Rb binding sdAb. CDRs obtained from antibodies, heavy chain antibodies, and sdAbs derived therefrom may be grafted onto alternative frameworks as described in Saerens, et al. (2005) J. Mol Biol 352:597-607 to generate CDR-grafted sdAbs. In some embodiments, the present disclosure provides a IL10Rb binding molecule comprising a CDR grafted IL10Rb binding sdAb, said CDR-grafted IL10Rb binding sdAb comprising a set of CDRs1, 2, and 3 as shown in a row of Table 2. In some embodiments, the present disclosure provides a IL10Rb binding molecule comprising a CDR grafted IL10Rb binding sdAb, said CDR-grafted IL10Rb binding sdAb comprising a set of CDRs1, 2, and 3 as shown in a row of Table 3.

Chimeric and Humanized sdAbs

Any framework region can be used with the CDRs as described herein. In some embodiments, the IL10Rb binding sdAb is a chimeric sdAb, in which the CDRs are derived from one species (e.g., camel) and the framework and/or constant regions are derived from another species (e.g., human or mouse). In specific embodiments, the framework regions are human or humanized sequences. Thus, humanized IL10Rb binding sdAbs derived from hIL10Rb binding VHHs are considered within the scope of the present disclosure. The techniques for humanization of camelid single domain antibodies are well known in the art. See, e.g., Vincke, et al. (2009) General Strategy to Humanize a Camelid Single-domain Antibody and Identification of a Universal Humanized Nanobody Scaffold J. Biol. Chem. 284(5)3273-3284.

In some embodiments, a VHH described herein can be humanized to contain human framework regions. Examples of human germlines that could be used to create humanized VHHs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

IFNlambda1 Receptor Subunit IL28RA

The IL28RA binding molecules of the present disclosure specifically bind to the extracellular domain of the IL28RA.

In one embodiment, specifically bind to the extracellular domain of the human IL28RA receptor subunit (hIL28RA). hIL28RA is expressed as a 520 amino acid precursor comprising a 20 amino acid N-terminal signal sequence which is post-translationally cleaved to provide a 500 amino acid mature protein. The canonical full-length acid hIL28RA precursor (including the signal peptide) is a 520 amino acid polypeptide having the amino acid sequence:

(SEQ ID NO: 5)
MAGPERWGPLLLCLLQAAPGLCSMMCLKKQDLYNKFKGRVRTVSPSSKSP
WVESEYLDYLFEVEPAPPVLVLTQTEEILSANATYQLPPCMPPLDLKYEV
AFWKEGAGNKTLFPVTPHGQPVQITLQPAASEHHCLSARTIYTFSVPKYS
KFSKPTCFLLEVPEANWAFLVLPSLLILLLVIAAGGVIWKTLMGNPWFQR
AKMPRALDFSGHTHPVATFQPSRPESVNDLFLCPQKELTRGVRPTPRVRA
PATQQTRWKKDLAEDEEEEDEEDTEDGVSFQPYIEPPSFLGQEHQAPGHS
EAGGVDSGRPRAPLVPSEGSSAWDSSDRSWASTVDSSWDRAGSSGYLAEK
GPGQGPGGDGHQESLPPPEFSKDSGFLEELPEDNLSSWATWGTLPPEPNL
VPGGPPVSLQTLTFCWESSPEEEEEARESEIEDSDAGSWGAESTQRTEDR
GRTLGHYMAR.

For purposes of the present disclosure, the numbering of amino acid residues of the IL28RA polypeptides as described herein is made in accordance with the numbering of this canonical sequence (UniProt Reference No U8IU57, SEQ ID NO:5). Amino acids 1-20 of SEQ ID NO:5 are identified as the signal peptide of hIL28R, amino acids 21-228 of SEQ ID NO:5 are identified as the extracellular domain, amino acids 229-249 of SEQ ID NO:5 are identified as the transmembrane domain, and amino acids 250-520 of SEQ ID NO:5 are identified as the intracellular domain.

For the purposes of generating antibodies that bind to the ECD of IL28R, immunization may be performed with the extracellular domain of the hIL28R. The extracellular domain of hIL28RA is a 208 amino acid polypeptide of the sequence:

(SEQ ID NO: 6)
RPRLAPPQNVTLLSQNFSVYLTWLPGLGNPQDVTYFVAYQSSPTRRRWRE
VEECAGTKELLCSMMCLKKQDLYNKFKGRVRTVSPSSKSPWVESEYLDYL
FEVEPAPPVLVLTQTEEILSANATYQLPPCMPPLDLKYEVAFWKEGAGNK
TLFPVTPHGQPVQITLQPAASEHHCLSARTIYTFSVPKYSKFSKPTCFLL
EVPEANWA.

### Cross Reactivity:

In some instances, due to sequence or structural similarities between the extracellular domains of IL28RA receptors from various mammalian species, immunization with an antigen derived from a IL28RA of a first mammalian species (e.g., the hIL28R-ECD, SEQ ID NO:72) may provide antibodies which specifically bind to IL28RA receptors of one or more additional mammalian species. Such antibodies are termed “cross reactive.” For example, immunization of a camelid with a human derived antigen (e.g., the hIL28R-ECD) may generate antibodies that are cross-reactive the murine and human receptors. Evaluation of cross-reactivity of antibody with respect to the receptors derived from other mammalian species may be readily determined by the skilled artisan, for example using the methods relating to evaluation of binding affinity and/or specific binding described elsewhere herein such as flow cytometry or SPR. Consequently, the use of the term “human IL28RA VHH” or “hIL28RA VHH” merely denotes that the species of the IL28RA antigen used for immunization of the camelid from which the VHH was derived was the human IL28RA (e.g., the hIL28RA ECD) but should not be understood as limiting with respect to the specific binding affinity of the VHH for IL28RA molecules of other mammalian species.

II. IL28RA Binding Molecules and Single Domain Antibodies

In some embodiments, a IL28RA binding molecule of the present disclosure is a single domain antibody (sdAb). The present disclosure relates to IL28RA binding molecules comprising single domain antibodies (sdAbs) that specifically bind to the extracellular domain of the human IL28RA isoform (hIL28R) which are found on all IL28R-expressing cells.

A single-domain antibody (sdAb) is an antibody containing a single monomeric variable antibody domain. Like a full-length antibody, sdAbs are able to bind specifically to an antigenic determinant. hIL28RA binding VHH single-domain antibodies can be engineered from heavy chain antibodies isolated from Camelidae mammals (e.g., camels, llamas, dromedary, alpaca, and guanaco) immunized with the extracellular domain of hIL28RA or an immunologically active fragment thereof. Descriptions of sdAbs and VHHs can be found in, e.g., De Greve et al., (2019) Curr Opin Biotechnol. 61:96-101; Ciccarese, et al., (2019) Front Genet. 10:997: Chanier and Chames (2019) Antibodies (Basel) 8(1); and De Vlieger, et al. (2018) Antibodies (Basel) 8(1). Alternatively, hIL28RA single domain antibodies may be engineered from heavy chain antibodies isolated from the IgNAR heavy chain antibodies isolated from cartilaginous fishes immunized with the extracellular domain of hIL28RA or an immunologically active fragment thereof hIL28RA binding sdAbs may also be obtained by splitting the dimeric variable domains from immunoglobulin G (IgG) isotypes from other mammalian species including humans, rats, rabbits immunized with the extracellular domain of hIL28RA or an immunologically active fragment thereof. Although most research into sdAbs is currently based on heavy chain variable domains, sdAbs derived from light chains have also been shown to bind specifically to the IL28RA proteins comprising the antigenic immunization sequence. Moller et al., J Biol Chem. 285(49):38348-38361, 2010.

In some embodiments, the sdAb is a VHH. A VHH is a type of sdAb that has a single monomeric heavy chain variable antibody domain. Similar to a traditional antibody, a VHH is able to bind specifically to a specific antigen. An exemplary VHH has a molecular weight of approximately 12-15 kDa which is much smaller than traditional mammalian antibodies (150-160 kDa) composed of two heavy chains and two light chains. VHHs can be found in or produced from Camelidae mammals (e.g., camels, llamas, dromedary, alpaca, and guanaco) which are naturally devoid of light chains.

The present disclosure provides IL28RA binding molecules comprising a polypeptide having at least 75%, alternatively 80%, alternatively 90%, alternatively 95%, alternatively 98%, or alternatively 99% or 100% identity to a polypeptide of any one of SEQ ID NOS:2-15.

The present disclosure provides IL28RA binding molecules comprising a CDR1, a CDR2, and a CDR3 as described in a row of Table 4 provided herein. In some embodiments, the CDR1, CDR2, and CDR3 can each, independently, comprise at least 90% (e.g., 91%, 92%, 93%, 94%, 95%, 96%, 97%, 98%, 99%, or 100%) sequence identity, or have 0, 1, 2, or 3 amino acid changes, optionally conservative amino acid changes, relative to the sequence described in a row of Table 4 provided herein.

Modified Forms of Single Domain Antibodies

CDR Grafted sdAbs

In some embodiments, the IL28RA binding sdAb of the present disclosure is a CDR grafted IL28RA binding sdAb. CDRs obtained from antibodies, heavy chain antibodies, and sdAbs derived therefrom may be grafted onto alternative frameworks as described in Saerens, et al. (2005) J. Mol Biol 352:597-607 to generate CDR-grafted sdAbs. In some embodiments, the present disclosure provides a IL28RA binding molecule comprising a CDR grafted IL28RA binding sdAb, said CDR-grafted IL28RA binding sdAb comprising a set of CDRs1, 2, and 3 as shown in a row of Table 4. In some embodiments, the present disclosure provides a IL28RA binding molecule comprising a CDR grafted IL28RA binding sdAb, said CDR-grafted IL28RA binding sdAb comprising a set of CDRs1, 2, and 3 as shown in a row of Table 4.

Elimination of N-Linked Glycosylation Sites

In some embodiments, it is possible that an amino acid sequence (particularly a CDR sequence) of the IL28RA binding sdAb may contain a glycosylation motif, particularly an N-linked glycosylation motif of the sequence Asn-X-Ser (N—X—S) or Asn-X-Thr (N—X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation. As procaryotic host cells do not provide the mechanism for glycosylation of recombinant proteins, when employing a procaryotic expression system to produce a recombinant IL28RA binding sdAb the modification of the sequence to eliminate the N-linked glycosylation sites may be obviated.

Chimeric and Humanized sdAbs

Any framework region can be used with the CDRs as described herein. In some embodiments, the IL28RA binding sdAb is a chimeric sdAb, in which the CDRs are derived from one species (e.g., camel) and the framework and/or constant regions are derived from another species (e.g., human or mouse). In specific embodiments, the framework regions are human or humanized sequences. Thus, humanized IL28RA binding sdAbs derived from hIL28RA binding VHHs are considered within the scope of the present disclosure. The techniques for humanization of camelid single domain antibodies are well known in the art. See, e.g., Vincke, et al. (2009) General Strategy to Humanize a Camelid Single-domain Antibody and Identification of a Universal Humanized Nanobody Scaffold J. Biol. Chem. 284(5)3273-3284.

In some embodiments, a VHH described herein can be humanized to contain human framework regions. Examples of human germlines that could be used to create humanized VHHs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

IL28A: the Cognate Ligand for the IL28RA

The cognate ligand for the IL28RA receptor is the cytokine IL28A (also known as Interferon lambda-2 or IFN lambda-2). IL28A is a homodimeric polypeptide which is an agonist of the class II cytokine receptor composed of IL10RB and IFNLR1/IL28RA. Human IL28A is a non-covalently linked homodimeric protein comprising two identical subunits. The canonical amino acid sequence of one subunit of the homodimeric human IL28A is provided below (UniProt Reference No: Q8IZJO)

(SEQ ID NO: 273)
MKLDMTGDCTPVLVLMAAVLTVTGAVPVARLHGALPDARGCHIAQFKSLS
PQELQAFKRAKDALEESLLLKDCRCHSRLFPRTWDLRQLQVRERPMALEA
ELALTLKVLEATADTDPALVDVLDQPLHTLHHILSQFRACIQPQPTAGPR
TRGRLHHWLYRLQEAPKKESPGCLEASVTFNLFRLLTRDLNCVASGDLCV

The canonical amino acid sequence of one subunit of the homodimeric mouse Interferon lambda-2/IL28A is provided below (UniProt Reference No: Q4VK74)

(SEQ ID NO: 274)
MLLLLLPLLLAAVLTRTQADPVPRATRLPVEAKDCHIAQFKSLSPKELQA
FKKAKDAIEKRLLEKDLRCSSHLFPRAWDLKQLQVQERPKALQAEVALTL
KVWENMTDSALATILGQPLHTLSHIHSQLQTCTQLQATAEPRSPSRRLSR
WLHRLQEAQSKETPGCLEASVTSNLFRLLTRDLKCVANGDQCV

III. IfnλR1 Receptor Binding Molecules

In another aspect, the disclosure provides an IFN2a1 (IFNlamdaR1) binding protein that specifically binds to IL10Rβ and IL28 receptor subunit A (IL28Rα), wherein the binding protein causes the multimerization of IL10Rβ and IL28Rα and downstream signaling, and wherein the binding protein comprises a single-domain antibody (sdAb) that specifically binds to IL10Rβ (an anti-IL10Rβ sdAb) and a sdAb that specifically binds to IL28Rα (an anti-IL28Rα sdAb).

In some embodiments, the anti-IL10Rβ sdAb is a VHH antibody (an anti-VHH antibody) and/or the anti-IL28Ra sdAb is a VHH antibody (an anti IL28Rα VHH antibody). In some embodiments, the anti-IL10R13 sdAb and the anti-IL28Ra sdAb are joined directly or via a peptide linker. In some embodiments, the peptide linker comprises between 1 and 50 amino acids.

In another aspect, the disclosure features a method for treating an infectious disease in a subject in need thereof, comprising administering to the subject an IFNλR binding protein described herein, wherein the IFINλR binding protein binds to and activates macrophages, CD8+ T cells, CD4+ T cells, Treg cells, dendritic cells, and/or epithelial cells. In some embodiments, the IFNλR binding protein binds to and activates macrophages. In some embodiments, the infectious disease is influenza, hepatitis B, hepatitis C, or human immunodeficiency virus (HIV) infection.

### IFN21 Receptor Binding Proteins

The interferon (IFN) λ receptor (IFN2a) includes IL10Rβ and IL28 receptor (IL28R) a subunit (IL28Rα). Provided herein is an IFNλR binding protein that specifically binds to IL10Rβ and IL28Rα. In some embodiments, the IFNλR binding protein binds to a mammalian cell expressing both IL10Rβ and IL28Rα. In some embodiments, the IFNλR binding protein can be a bispecific VHH 2 as described below. In other embodiments, the IFNλR binding protein can include a first domain that is a VHH and a second domain which can be a fragment of IFNλ1 or, for example, a scFv.

The IFNλR binding protein can be a bispecific VHH 2 that has a first VHH binding to IL10Rβ (an anti-IL10Rβ VHH antibody) and a second VHH binding to IL28Rα (an anti-IL28Rα VHH antibody) and causes the dimerization of the two receptor subunits and downstream signaling when bound to a cell expressing IL10Rβ and IL28R, e.g., a macrophage, a T cell (e.g., a CD8⁺ T cell or a CD4⁺ T cell), a Treg cell, a dendritic cell, and/or an epithelial cell.

A linker can be used to join the anti-IL10Rβ VHH antibody and the anti-IL28Rα VHH antibody. For example, a linker can simply be a covalent bond or a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). A peptide linker joining the anti-IL10R13 VHH antibody and the anti-IL28Rα VHH antibody can be a flexible glycine-serine linker. A linker can also be a chemical linker, such as a synthetic polymer, e.g., a polyethylene glycol (PEG) polymer.

The anti-IL10RP VHH antibody can have a sequence having at least 90% (e.g., 91%, 92%, 93%, 94%, 95%, 96%, 97%, 98%, 99%, or 100%) sequence identity to one or more sequences in Table 5 or Table 6.

The anti-IL28Rα VHH antibody can have a sequence having at least 90% (e.g., 91%, 92%, 93%, 94%, 95%, 96%, 97%, 98%, 99%, or 100%) sequence identity to one or more sequences in Table 7.

In some embodiments, the IFNλR1 binding protein has a reduced Eₘₐₓ compared to the Eₘₐₓ caused by IFNλ1. Eₘₐₓ reflects the maximum response level in a cell type that can be obtained by a ligand (e.g., a binding protein described herein or the native cytokine (e.g., IFNλ). In some embodiments, the IFNλR1 binding protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IFNλ1. In other embodiments, the Eₘₐₓ of the IFNλR1 binding protein described herein is greater (e.g., at least 1%, 5%, 10%, 15%, 20%, 25%, 30%, 35%, 40%, 45%, or 50% greater) than the Eₘₐₓ of the natural ligand, IFNλ1. In some embodiments, by varying the linker length of the IFNλR1 binding protein, the Eₘₐₓ of the IFNλR binding protein can be changed. The IFNλR1 binding protein can cause Eₘₐₓ in the most desired cell types (e.g., macrophages), and a reduced Eₘₐₓ in other cell types.

The IFNλR1 binding proteins of the present disclosure are useful in the treatment of an infectious disease in a subject in need thereof. The IFNλR binding protein binds to and activates macrophages, CD8⁺ T cells, CD4⁺ T cells, Treg cells, dendritic cells, and/or epithelial cells. In particular, the IFNλR binding protein binds to and activates macrophages. Examples of infectious diseases include, but are not limited to, influenza, hepatitis B, hepatitis C, and human immunodeficiency virus (HIV) infection. In some embodiments, the IFNλR binding protein can protect Kuppfer cells in the liver against the effects of an infectious disease. The IFNλR binding protein can trigger different levels of downstream signaling in different cell types. For example, by varying the length of the linker between the anti-IL10R13 VHH antibody and the anti-IL28Rα V_{H}H antibody in the IFNλR binding protein, the IFNλR binding protein can cause a higher level of downstream signaling in desired cell types (e.g., macrophages) compared to undesired cell types. In some embodiments, by varying the linker length, an IFNλR binding protein results in the modulation of downstream signaling in macrophages compared to the level of downstream signaling in other cell types. In other embodiments, different anti-IL10Rβ VHH antibodies with different binding affinities and different anti-IL28Rα V_{H}H antibodies with different binding affinities can be combined to make different IFNλR binding proteins. Further, the orientation of the two antibodies in the binding protein can also be changed to make a different binding protein (i.e., anti-IL10Rβ VHH antibody-linker-anti-IL28Rα VHH antibody, or anti-IL28Rα VHH antibody-linker-anti-IL10Rβ V_{H}H antibody). Different IFNλR binding proteins can be screened to find the ideal binding protein that causes a higher level of downstream signaling in desired cell types compared to undesired cell types. In some embodiments, the level of downstream signaling in macrophages is at least 1.1, 1.5, 2, 3, 5, or 10 times of the level of downstream signaling in other cell types.

### Single Domain Antibody Is A VHH

In some embodiments, the single domain antibody is a VHH. A V_{H}H is a type of single-domain antibody (sdAb) containing a single monomeric variable antibody domain. Like a full-length antibody, it is able to bind selectively to a specific antigen. The complementary determining regions (CDRs) of V_{H}Hs are within a single-domain polypeptide. V_{H}Hs can be engineered from heavy-chain antibodies found in camelids. An exemplary VHH has a molecular weight of approximately 12-15 kDa which is much smaller than traditional mammalian antibodies (150-160 kDa) composed of two heavy chains and two light chains. V_{H}Hs can be found in or produced from Camelidae mammals (e.g., camels, llamas, dromedary, alpaca, and guanaco) which are naturally devoid of light chains. Descriptions of sdAbs and VHHS can be found in, e.g., De Greve et al., Curr Opin Biotechnol. 61:96-101, 2019; Ciccarese, et al., Front Genet. 10:997, 2019; Chanier and Chames, Antibodies (Basel) 8(1), 2019; and De Vlieger et al., Antibodies (Basel) 8(1), 2018.

### Exemplary Anti IL28Rα Single Domain Antibodies

Table 4 provides CDRs useful in the preparation of anti-IL28RA sdAbs. In some embodiments, the anti-IL28RA sdAbs is a single domain antibody comprising, with reference to the CDRs provided in Table 4: a CDR1 having 0, 1, 2, or 3 amino acid changes relative to the sequence of any one of the CDR's in Table 4; a CDR2 having 0, 1, 2, or 3 amino acid changes relative to the sequence of any one of the CDR2s in Table 4; and a CDR3 having 0, 1, 2, or 3 amino acid changes relative to the sequence of any one of the CDR3s in Table 4.

In some embodiments, the anti-IL28RA sdAb comprises a VHH sequence having at least 90% (e.g., 91%, 92%, 93%, 94%, 95%, 96%, 97%, 98%, 99%, or 100%) sequence identity to a sequence of any one the of anti-IL28RA sdAbs provided in Table 7. In certain embodiments, the binding molecule comprises a sequence that is substantially identical to a sequence of any one of the sequences listed in a row of Table 7.

In another aspect, the disclosure provides an isolated nucleic acid encoding an anti-IL28RA sdAb described herein. Table 10 provides DNA sequences encoding the anti-IL10Rb sdAbs of Table 7. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence listed in a row of Table 10.

### Anti IFNlambdaR1 VHH Dimer Bispecific Binding Molecules

### A. “Forward Orientation”

In some embodiments, the bivalent IFNlambdaR1 binding molecule comprises a polypeptide of the structure:

H₂N-[anti-IL10Rb sdAb]-[L]ₓ-[anti-IL28RA sdAb]-[TAG]_{y}—COOH

wherein L is a polypeptide linker of 1-50 amino acids and x=0 or 1, and TAG is a chelating peptide or a subunit of an Fc domain and y=0 or 1.

In some embodiments, a bivalent IFNlambdaR1 binding molecule of the foregoing structure comprises a polypeptide from amino to carboxy terminus:
(a) an anti-IL10Rb sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes, relative to the sequence of any one of the CDR1s in Table 2 or 3.
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR2s in Table 2 or 3; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR3s in Table 2 or 3;
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an anti-IL28RA sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR1s in Table 4;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR2s in Table 4; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR3s in Table 4.

In some embodiments, the bivalent IFNlambdaR1 binding molecule of the “forward” configuration comprises an anti-IL10Rb sdAb comprising a CDR1, a CDR2, and a CDR3 listed in a row of Tables 2 or 3, and an anti-IL28RA sdAb comprising a CDR1, a CDR2, and a CDR3 as listed in a row of Table 4, including any combination thereof.

In some embodiments, the IFNlambdaR1 binding molecule of the “forward” configuration comprises a polypeptide from amino to carboxy terminus:
(a) an IL10rb sdAb comprising an amino acid sequence having at least 90% (e.g., 91%, 92%, 93%, 94%, 95%, 96%, 97%, 98%, 99%, or 100%) sequence identity to a sequence of any one of the VHH sequences in Table 5 or 5;
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an IL28RA sdAb comprising a sequence having at least 90% (e.g., 91%, 92%, 93%, 94%, 95%, 96%, 97%, 98%, 99%, or 100%) sequence identity to a sequence of any one of the VHH sequences in Table 7.

In some embodiments, the anti-IL10Rb sdAb of the bivalent IFNlambdaR1 binding molecule of the “forward” configuration comprises a VHH sequence having at least 90% (e.g., 91%, 92%, 93%, 94%, 95%, 96%, 97%, 98%, 99%, or 100%) sequence identity to a sequence of any one the of anti-IL10Rb sdAbs provided in Table 5 or 6. In some embodiments, the anti-IL28RA sdAb of the bivalent IFNlambdaR1 binding molecule comprises a VHH sequence having at least 90% (e.g., 91%, 92%, 93%, 94%, 95%, 96%, 97%, 98%, 99%, or 100%) sequence identity to a sequence of any one the of anti-IL28RA sdAbs provided in Table 7.

In some embodiments of the forward configuration, the bivalent IFNLR binding molecule comprises an IL10Rb sdAb sdAb in combination with an anti-IL28RA sdAb. In some embodiments, the bivalent IFNLR binding molecule comprises an anti-IL10Rb sdAb comprising an amino acid sequence having at least 90% (e.g., 91%, 92%, 93%, 94%, 95%, 96%, 97%, 98%, 99%, or 100%) identity to a sequence listed in a row of Table 5 or 6 in combination with an anti-IL28RA sdAb comprising an amino acid sequence having at least 90% (e.g., 91%, 92%, 93%, 94%, 95%, 96%, 97%, 98%, 99%, or 100%) identity to a sequence listed in a row of Table 7.

### B. “Reverse Orientation”

In some embodiments, the bivalent IFNlambdaR1 binding molecule comprises a polypeptide of the structure:

H₂N-[anti-IL28RA sdAb]-[L]ₓ-[anti-IL10Rb sdAb]-[TAG]_{y}—COOH

wherein and L is a polypeptide linker of 1-50 amino acids and x=0 or 1, and TAG is a chelating peptide or a subunit of an Fc domain and y=0 or 1.

In some embodiments, a bivalent IFNlambdaR1 binding molecule of the foregoing structure comprises a polypeptide from amino to carboxy terminus:
(a) an anti-IL28RA sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR1s in Table 4;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR2s in Table 4; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR3s in Table 4;
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an anti-IL10Rb sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR1s in Table 2 or 3;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR2s in Table 2 or 3; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of the CDR3s in Table 2 or 3.

In some embodiments of the reverse orientation, the anti-IL28RA sdAb comprises a VHH sequence having at least 90% (e.g., 91%, 92%, 93%, 94%, 95%, 96%, 97%, 98%, 99%, or 100%) sequence identity to a sequence listed in a row of Table 7. In certain embodiments, the anti-IL10Rb sdAb comprises a VHH sequence having at least 90% sequence identity to a sequence of any one of listed in a row of Table 5 or 6.

In some embodiments of the reverse orientation, the bivalent IFNLR binding molecule comprises an IL28RA sdAb in combination with an anti-IL10Rb sdAb. In some embodiments, the bivalent IFNLR binding molecule comprises an anti-IL28RA sdAb comprising an amino acid sequence having at least 90% (e.g., 91%, 92%, 93%, 94%, 95%, 96%, 97%, 98%, 99%, or 100%) identity to a sequence listed in a row of Table 7 in combination with an anti-IL10Rb sdAb comprising an amino acid sequence having at least 90% (e.g., 91%, 92%, 93%, 94%, 95%, 96%, 97%, 98%, 99%, or 100%) identity to a sequence listed in a row of Table 5 or 6.

A linker can also be a chemical linker, such as a synthetic polymer, e.g., a polyethylene glycol (PEG) polymer.

The length of the linker between two sdAb in a binding molecule can be used to modulate the proximity of the two sdAb of the binding molecule. By varying the length of the linker, the overall size and length of the binding molecule can be tailored to bind to specific cell receptors or domains or subunits thereof. For example, if the binding molecule is designed to bind to two receptors or domains or subunits thereof that are located close to each other on the same cell, then a short linker can be used. In another example, if the binding molecule is designed to bind to two receptors or domains or subunits there of that are located on two different cells, then a long linker can be used.

In some embodiments, a linker joins the C-terminus of the anti-IL10Rb sdAb in the binding molecule to the N-terminus of the anti-IL28RA sdAb in the binding molecule. In other embodiments, a linker joins the C-terminus of the anti-IL28RA sdAb in the binding molecule to the N-terminus of the anti-IL10Rb sdAb in the binding molecule.

Modulation of sdAb Binding Affinity:

In some embodiments, the activity and/or specificity of the bivalent IFNlambdaR1 binding molecule of the present disclosure may be modulated by the respective binding affinities of the sdAbs for their respective receptor subunits.

It will be appreciated by one of skill in the art that the binding of the first sdAb of the bivalent IFNlambdaR1 binding molecule to the first receptor subunit ECD on the cell surface will enhance the probability of a binding interaction between the second sdAb of the bivalent IFNlambdaR1 binding molecule with the ECD of the second receptor subunit. This cooperative binding effect may result in a bivalent IFNlambdaR1 binding molecule which has a very high affinity for the receptor and a very slow “off rate” from the receptor. Typical VHH single domain antibodies have an affinity for their targets of from about 10⁻⁵ M to about 10⁻¹⁰ M. In those instances such slow off-rate kinetics are desirable in the bivalent IFNlambdaR1 binding molecule, the selection of sdAbs having high affinities (about 10⁻⁷ M to about 10⁻¹⁰ M) for incorporation into the bivalent IFNlambdaR1 binding molecule are favored.

Naturally occurring cytokine ligands for typically do not exhibit a similar affinity for each subunit of a heterodimeric receptor. Consequently, in designing a bivalent IFNlambdaR1 binding molecule which is a mimetic of the cognate cytokine IFNlambda as contemplated by some embodiments of the present disclosure, selection of sdAbs for the first and second IFNlambdaR1 receptor subunit have an affinity similar to (e.g., having an affinity about 10 fold, alternatively about 20 fold, or alternatively about fold higher or lower than) the cognate IFNlambda for the respective receptor subunit may be used.

In some embodiments, the bivalent IFNlambdaR1 binding molecules of the present disclosure are partial agonists of the IFNlambdaR1 receptor. As such, the activity of the bivalent binding molecule may be modulated by selecting sdAb which have greater or lesser affinity for either one or both of the IFNlambdaR1 receptor subunits. As some heterodimeric cytokine receptors are comprised of a “proprietary subunit” (i.e., a subunit which is not naturally a subunit of another multimeric receptor) and a second “common” subunit (such as CD132) which is a shared component of multiple cytokine receptors), selectivity for the formation of such receptor may be enhanced by employing first sdAb which has a higher affinity for the proprietary receptor subunit and second sdAB which exhibits a lower affinity for the common receptor subunit. Additionally, the common receptor subunit may be expressed on a wider variety of cell types than the proprietary receptor subunit. In some embodiments wherein the receptor is a heterodimeric receptor comprising a proprietary subunit and a common subunit, the first sdAb of the bivalent IFNlambdaR1 binding molecule exhibits a significantly greater (more than 10 times greater, alternatively more than 100 times greater, alternatively more than 1000 times greater) affinity for the proprietary receptor than the second sdAb of the bivalent IFNlambdaR1 binding molecule for the common receptor subunit. In one embodiment, the present disclosure provides a bivalent IFNlambdaR1 binding molecule wherein the affinity of the anti-IL10Rb sdAb of has an affinity of more than 10 times greater, alternatively more than 100 times greater, alternatively more than 1000 times greater) affinity anti-IL28RA sdAb common receptor subunit.

III. Modifications to Extend Duration of Action In Vivo

The IFNlambdaR1 bivalent binding molecule described herein can be modified to provide for an extended lifetime in vivo and/or extended duration of action in a subject. In some embodiments, the binding molecule can be conjugated to carrier molecules to provide desired pharmacological properties such as an extended half-life. In some embodiments, the binding molecule can be covalently linked to the Fc domain of IgG, albumin, or other molecules to extend its half-life, e.g., by pegylation, glycosylation, and the like as known in the art. In some embodiments, the IFNlambdaR1 bivalent binding molecule modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the IFNlambdaR1 bivalent binding molecule to provide an extended duration of action in a mammalian subject include (but are not limited to);
conjugation of the IFNlambdaR1 bivalent binding molecule to one or more carrier molecules,
conjugation IFNlambdaR1 bivalent binding molecule to protein carriers molecules, optionally in the form of a fusion protein with additional polypeptide sequences (e.g, IFNlambdaR1 bivalent binding molecule-Fc fusions) and
conjugation to polymers, (e.g. water soluble polymers to provide a PEGylated IFNlambdaR1 bivalent binding molecule).

It should be noted that the more than one type of modification that provides for an extended duration of action in a mammalian subject may be employed with respect to a given IFNlambdaR1 bivalent binding molecule. For example, IFNlambdaR1 bivalent binding molecule of the present disclosure may comprise both amino acid substitutions that provide for an extended duration of action as well as conjugation to a carrier molecule such as a polyethylene glycol (PEG) molecule.

### Protein Carrier Molecules:

Examples of protein carrier molecules which may be covalently attached to the IFNlambdaR1 bivalent binding molecule to provide an extended duration of action in vivo include, but are not limited to albumins, antibodies and antibody fragments such and Fc domains of IgG molecules

### Fc Fusions:

In some embodiments, the IFNlambdaR1 bivalent binding molecule is conjugated to a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

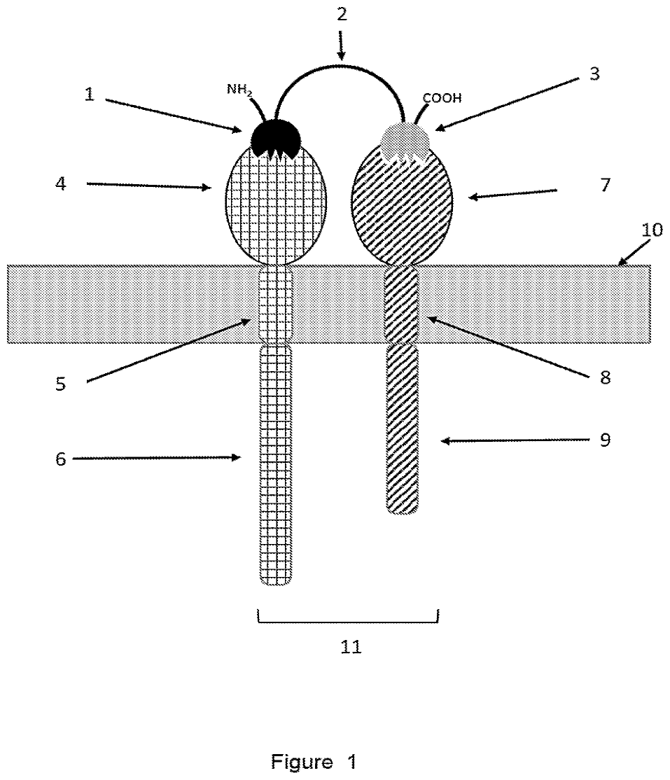

Illustrative examples of Fc formats useful for IFNlambdaR1 bivalent binding molecules of the present disclosure are provided schematically in FIGS. 1-4 of the attached drawings.

### Linkage of Bivalent Binding Molecule to Fc

As indicated, the linkage of the IFNlambdaR1 bivalent binding molecule to the Fc subunit may incorporate a linker molecule as described below between the bivalent sdAb and Fc subunit. In some embodiments, the IFNlambdaR1 bivalent binding molecule is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fc domains may be derived from human IgG1, IgG2, IgG3 and IgG4 IgG species. In some embodiments, the hinge region is the hinge region of an IgG1. In one particular embodiment, the IFNlambdaR1 bivalent binding is linked to an Fc domain using an human IgG1 hinge domain.

### Knob-Into-Hole Fc Format

In some embodiments, when the IFNlambdaR1 bivalent binding molecule described herein is to be administered in the format of an Fc fusion, particularly in those situations when the polypeptide chains conjugated to each subunit of the Fc dimer are different, the Fc fusion may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”), and ii) in the CH3 domain of a second heavy chain, an amino acid residue is replaced with an amino acid residue having a smaller side chain (e.g., alanine or threonine), thereby generating a cavity (“hole”) at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 in one chain and Y349 in the other chain which results in a stabilizing disulfide bridge between the two antibody heavy chains in the Fc region (Carter, et al. (2001) Immunol Methods 248, 7-15).

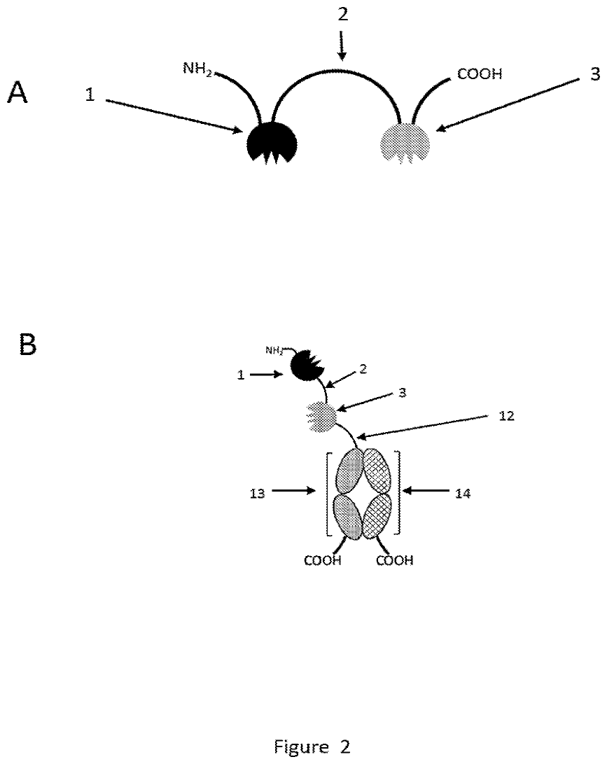

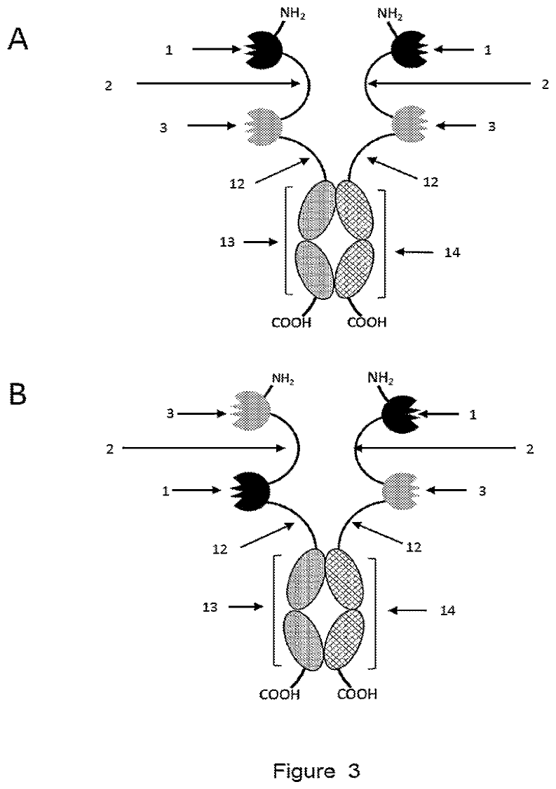

The knob-into-hole format is used to facilitate the expression of a first polypeptide on a first Fc monomer with a “knob” modification and a second polypeptide on the second Fc monomer possessing a “hole” modification to facilitate the expression of heterodimeric polypeptide conjugates. In some embodiments, the IFNlambdaR1 binding molecule is covalently linked to a single subunit of the Fc as illustrated in FIG. 2. In some embodiments, the IFNlambdaR1 bivalent binding molecule is provided on each of the subunits of the Fc as illustrated in FIG. 3.

### Albumin Carrier Molecules

In some embodiments, the IFNlambdaR1 bivalent binding molecule conjugated to an is albumin molecule (e.g., human serum albumin) which is known in the art to facilitate extended exposure in vivo. In one embodiment of the invention, the IFNlambdaR1 bivalent binding molecule is conjugated to albumin via chemical linkage or expressed as a fusion protein with an albumin molecule referred to herein as an IFNlambdaR1 bivalent binding molecule albumin fusion.” The term “albumin” as used in the context αβhIL2 mutein albumin fusions include albumins such as human serum albumin (HSA), cyno serum albumin, and bovine serum albumin (BSA). In some embodiments, the HSA the HSA comprises a C34S or K573P amino acid substitution relative to the wild-type HSA sequence According to the present disclosure, albumin can be conjugated to a IFNlambdaR1 bivalent binding molecule at the carboxyl terminus, the amino terminus, both the carboxyl and amino termini, and internally (see, e.g., U.S. Pat. Nos. 5,876,969 and 7,056,701). In the HAS IFNlambdaR1 bivalent binding molecule contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a IFNlambdaR1 bivalent binding molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the IFNlambdaR1 bivalent binding molecule and the albumin molecule the IFNlambdaR1 bivalent binding molecule—albumin complex may be provided as a fusion protein comprising an albumin polypeptide sequence and an IFNlambdaR1 bivalent binding molecule recombinantly expressed in a host cell as a single polypeptide chain, optionally comprising a linker molecule between the albumin and IFNlambdaR1 bivalent binding molecule. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein solated from the host cell culture by techniques well known in the art.

### Polymeric Carriers

In some embodiments, extended in vivo duration of action of the IFNlambdaR1 bivalent binding molecule may be achieved by conjugation to one or more polymeric carrier molecules such as XTEN polymers or water soluble polymers.

XTEN Conjugates

The IFNlambdaR1 bivalent binding molecule may further comprise an XTEN polymer. The XTEN polymer may be is conjugated (either chemically or as a fusion protein) the αβhIL2 mutein provides extended duration of akin to PEGylation and may be produced as a recombinant fusion protein in E. coli. XTEN polymers suitable for use in conjunction with the IFNlambdaR1 bivalent binding molecule of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE|DOI:10.1371/journal.pone.0157193 Jun. 13, 2016. The XTEN polymer may fusion protein may incorporate a protease sensitive cleavage site between the XTEN polypeptide and the hIL2 mutein such as an MMP-2 cleavage site.

Water Soluble Polymers

In some embodiments, the IFNlambdaR1 bivalent binding molecule can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol), polysaccharides), poly-alpha-hydroxy acid), polyvinyl alcohol (PVA), polyphosphazene, polyoxazolines (POZ), poly(N-acryloylmorpholine), or a combination thereof.

In some embodiments, IFNlambdaR1 bivalent binding molecule can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula

R(O—CH₂—CH₂)ₙO—R,

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

In some embodiments, selective PEGylation of the IFNlambdaR1 bivalent binding molecule, for example, by the incorporation of non-natural amino acids having side chains to facilitate selective PEG conjugation, may be employed. Specific PEGylation sites can be chosen such that PEGylation of the binding molecule does not affect its binding to the target receptors.

In some instances, the sequences of IFNlambdaR1 bivalent binding molecules provided in Tables 5, 6 and 7 of the present disclosure possess an N-terminal glutamine (“1Q”) residue. N-terminal glutamine residues have been observed to spontaneously cyclyize to form pyroglutamate (pE) at or near physiological conditions. (See e.g., Liu, et al (2011) J. Biol. Chem. 286(13): 11211-11217). In some embodiments, the formation of pyroglutamate complicates N-terminal PEG conjugation particularly when aldehyde chemistry is used for N-terminal PEGylation. Consequently, when PEGylating the IFNlambdaR1 binding molecules of the present disclosure, particularly when aldehyde chemistry is to be employed, the IFNlambdaR1 binding molecules possessing an amino acid at position 1 (e.g., 1Q) are substituted at position 1 with an alternative amino acid or are deleted at position 1 (e.g., des-1Q). In some embodiments, the IFNlambdaR1 binding molecules of the present disclosure comprise an amino acid substitution selected from the group Q1E and Q1D.

In certain embodiments, the increase in half-life is greater than any decrease in biological activity. PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about from about 10 kDa to about 20 kDa, from about 10 kDa to about 25 kDa, or from about to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a branched PEG comprising two 20 kD arms.

The present disclosure also contemplates compositions of conjugates wherein the PEGs have different n values, and thus the various different PEGS are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbonst

Two widely used first generation activated monomethoxy PEGs (mPEGs) are succinimidyl carbonate PEG (SC-PEG; see, e.g., Zalipsky, et al. (1992) Biotechnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear PEG-NHS ester.

In some embodiments, a linker can used to join the IFNlambdaR1 bivalent binding molecule and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl CoA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942):834-840.

### Fatty Acid Carriers

In some embodiments an IFNlambdaR1 bivalent binding molecule having an extended duration of action in a mammalian subject and useful in the practice of the present disclosure is achieved by covalent attachment of the IFNlambdaR1 bivalent binding molecule to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the IFNlambdaR1 bivalent binding molecule is acetylated at the N-terminus by enzymatic reaction with N-terminal acetyltransferase and, for example, acetyl CoA. Alternatively, or in addition to N-terminal acetylation, the IFNlambdaR1 bivalent binding molecule is acetylated at one or more lysine residues, e.g., by enzymatic reaction with a lysine acetyltransferase. See, for example Choudhary et al. (2009) Science 325 (5942):834L2 ortho840.

Modifications to Provide Additional Functions

In some embodiments, embodiment, the IFNlambdaR1 bivalent binding molecule may comprise a functional domain of a chimeric polypeptide. IFNlambdaR1 bivalent binding molecule fusion proteins of the present disclosure may be readily produced by recombinant DNA methodology by techniques known in the art by constructing a recombinant vector comprising a nucleic acid sequence comprising a nucleic acid sequence encoding the IFNlambdaR1 bivalent binding molecule in frame with a nucleic acid sequence encoding the fusion partner either at the N-terminus or C-terminus of the IFNlambdaR1 bivalent binding molecule, the sequence optionally further comprising a nucleic acid sequence in frame encoding a linker or spacer polypeptide.

### FLAG Tags

In other embodiments, the IFNlambdaR1 bivalent binding molecule can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

### Chelating Peptides

In some embodiment, the IFNlambdaR1 bivalent binding molecule (including fusion proteins of the IFNlambdaR1 bivalent binding molecule) of the present disclosure are may be covalently bonded via a peptide bond to one or more transition metal chelating polypeptide sequences. The association of the IFNlambdaR1 bivalent binding molecule with chelating peptide provides multiple utilities including: the purification of the IFNlambdaR1 bivalent binding molecule using immobilized metal affinity chromatography (IMAC) as described in Smith, et al. U.S. Pat. No. 4,569,794 issued Feb. 11, 1986; immobilization of the IFNlambdaR1 bivalent binding molecule on nitrilotriacetic acid (NTA) modified surface plasmon resonance sensor chips (e.g., Sensor Chip NTA available from Cytiva Global Life Science Solutions USA LLC, Marlborough MA as catalog number BR100407) as described in Nieba, et al. (1997) Analytical Biochemistry 252(2):217-228, or to form kinetically inert or kinetically labile complexes between the IFNlambdaR1 bivalent binding molecule and a transition metal ion as described in Anderson, et al. (U.S. Pat. No. 5,439,829 issued Aug. 8, 1995 and Hale, J. E (1996) Analytical Biochemistry 231(1):46-49. Examples of transition metal chelating polypeptides useful in the practice of the present disclosure are described in Smith, et al. supra and Dobeli, et al. U.S. Pat. No. 5,320,663 issued May 10, 1995 the entire teachings of which are hereby incorporated by reference. Particular transition metal chelating polypeptides useful in the practice of the present disclosure are peptides comprising 3-6 contiguous histidine residues (SEQ ID NO: 275) such as a six-histidine peptide (His)₆ (SEQ ID NO: 260) anTd are frequently referred to in the art as “His-tags.” In some embodiments, a purification handle is a polypeptide having the sequence Ala-Ser-His-His-His-His-His-His (“ASH6”) (SEQ ID NO: 276) or Gly-Ser-His-His-His-His-His-His-His-His (“GSH8”) (SEQ ID NO: 277).

### Targeting Moieties:

In some embodiments, IFNlambdaR1 bivalent binding molecule is conjugated to molecule which provides (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between IFNlambdaR1 bivalent binding molecule sequence and the sequence of the targeting domain of the fusion protein.

In other embodiments, a chimeric polypeptide including a IFNlambdaR1 bivalent binding molecule and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting moiety is an antibody that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3Ra2, CD19, mesothelin, Her2, EpCam, Muc1, ROR1, CD133, CEA, EGRFRVIII, PSCA, GPC3, Pan-ErbB and FAP.

Recombinant Production

Alternatively, the IFNlambdaR1 binding molecules of the present disclosure are produced by recombinant DNA technology. In the typical practice of recombinant production of polypeptides, a nucleic acid sequence encoding the desired polypeptide is incorporated into an expression vector suitable for the host cell in which expression will be accomplish, the nucleic acid sequence being operably linked to one or more expression control sequences encoding by the vector and functional in the target host cell. The recombinant protein may be recovered through disruption of the host cell or from the cell medium if a secretion leader sequence (signal peptide) is incorporated into the polypeptide.

Construction of Nucleic Acid Sequences Encoding the IFNlambdaR1 Binding Molecule

In some embodiments, the IFNlambdaR1 binding molecule is produced by recombinant methods using a nucleic acid sequence encoding the IFNlambdaR1 binding molecule (or fusion protein comprising the IFNlambdaR1 binding molecule). The nucleic acid sequence encoding the desired αβhIFNlambdaR1 binding molecule can be synthesized by chemical means using an oligonucleotide synthesizer.

The nucleic acid molecules are not limited to sequences that encode polypeptides;

some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the IFNlambdaR1 binding molecule (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IFNlambdaR1 binding molecule may be obtained from various commercial sources that provide custom made nucleic acid sequences. Amino acid sequence variants of the IFNlambdaR1 binding molecules of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding a IFNlambdaR1 binding molecule and expressing those sequences in a suitably transformed host include, but are not limited to, using a PCR-assisted mutagenesis technique. Mutations that consist of deletions or additions of amino acid residues to a IFNlambdaR1 binding molecule can also be made with standard recombinant techniques. In the event of a deletion or addition, the nucleic acid molecule encoding a IFNlambdaR1 binding molecule is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A IFNlambdaR1 binding molecule of the present disclosure may be produced recombinantly not only directly, but also as a fusion polypeptide with a heterologous polypeptide, e.g. a signal sequence or other polypeptide having a specific cleavage site at the N-terminus or C-terminus of the mature IFNlambdaR1 binding molecule. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the IFNlambdaR1 binding molecule from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the IFNlambdaR1 binding molecule into the culture medium as described in Singh, U.S. Pat. No. 7,198,919 B1 issued Apr. 3, 2007.

In the event the IFNlambdaR1 binding molecule to be expressed is to be expressed as a chimera (e.g., a fusion protein comprising a IFNlambdaR1 binding molecule and a heterologous polypeptide sequence), the chimeric protein can be encoded by a hybrid nucleic acid molecule comprising a first sequence that encodes all or part of the IFNlambdaR1 binding molecule and a second sequence that encodes all or part of the heterologous polypeptide. For example, subject IFNlambdaR1 binding molecules described herein may be fused to a hexa-/octa-histidine tag (SEQ ID NOS 260-261, respectively) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the IFNlambdaR1 binding molecule. For example, the N-terminus may be linked to a targeting domain and the C-terminus linked to a hexa-histidine tag (SEQ ID NO: 260) purification handle.

The complete amino acid sequence of the polypeptide (or fusion/chimera) to be expressed can be used to construct a back-translated gene. A DNA oligomer containing a nucleotide sequence coding a IFNlambdaR1 binding molecule can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the IFNlambdaR1 binding molecule may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Expression Vectors:

Once assembled (by synthesis, site-directed mutagenesis or another method), the nucleic acid sequence encoding an a IFNlambdaR1 binding molecule will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker:

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences:

Expression vectors for a IFNlambdaR1 binding molecules of the present disclosure contain a regulatory sequence that is recognized by the host organism and is operably linked to nucleic acid sequence encoding the IFNlambdaR1 binding molecule. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject a IFNlambdaR1 binding molecule, particularly as regards potential secondary structures.

### Promoters:

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers:

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells:

The present disclosure further provides prokaryotic or eukaryotic cells that contain and express a nucleic acid molecule that encodes a a IFNlambdaR1 binding molecule. A cell of the present disclosure is a transfected cell, i.e., a cell into which a nucleic acid molecule, for example a nucleic acid molecule encoding a mutant IL-2 polypeptide, has been introduced by means of recombinant DNA techniques. The progeny of such a cell are also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant IFNlambdaR1 binding molecule can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The IFNlambdaR1 binding molecule may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, a IFNlambdaR1 binding molecule obtained will be glycosylated or unglycosylated depending on the host organism used to produce the mutein. If bacteria are chosen as the host then the a IFNlambdaR1 binding molecule produced will be unglycosylated. Eukaryotic cells, on the other hand, will typically result in glycosylation of the IFNlambdaR1 binding molecule.

In some embodiments, it is possible that an amino acid sequence (particularly a CDR sequence) of an sdAb to be incorporated into a bivalent IFNlambdaR1 binding molecule may contain a glycosylation motif, particularly an N-linked glycosylation motif of the sequence Asn-X-Ser (N—X—S) or Asn-X-Thr (N—X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation motif.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif).

### Transfection:

The expression constructs of the can be introduced into host cells to thereby produce a IFNlambdaR1 binding molecule disclosed herein. The expression vector comprising a nucleic acid sequence encoding IFNlambdaR1 binding molecule is introduced into the prokaryotic or eukaryotic host cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins:

Recombinantly produced IFNlambdaR1 binding molecule polypeptides can be recovered from the culture medium as a secreted polypeptide if a secretion leader sequence is employed. Alternatively, the IFNlambdaR1 binding molecule polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinantly IFNlambdaR1 binding molecule by the transformed host can be purified according to any suitable method. Recombinant IFNlambdaR1 binding molecules can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant a IFNlambdaR1 binding molecule can be purified from the expression system using routine biochemical procedures, and can be used, e.g., as therapeutic agents, as described herein.

In some embodiments, where the IFNlambdaR1 binding molecule is expressed with a purification tag as discussed above, this purification handle may be used for isolation of the IFNlambdaR1 binding molecule from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794.

The biological activity of the IFNlambdaR1 binding molecules recovered can be assayed for activating by any suitable method known in the art and may be evaluated as substantially purified forms or as part of the cell lysate or cell medium when secretion leader sequences are employed for expression.

Pharmaceutical Formulations

In some embodiments, the subject IFNlambdaR1 binding molecule (and/or nucleic acids encoding the IFNlambdaR1 binding molecule or recombinant cells incorporating a nucleic acid sequence and modified to express the IFNlambdaR1 binding molecule) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the IFNlambdaR1 binding molecule is to be administered to the subject in need of treatment or prophyaxis.

### Carriers:

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor ELTM (BASF, Parsippany, N.J.) or phosphate buffered saline (PBS).

### Buffers:

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

### Dispersions:

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives:

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfate; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents:

In many cases, it will be preferable to include isotonic agents, for example, sugars, polyalcohols such as mannitol, sorbitol, sodium chloride in the composition.

Routes of Administration:

In some embodiments of the therapeutic methods of the present disclosure involve the administration of a pharmaceutical formulation comprising a IFNlambdaR1 binding molecule (and/or nucleic acids encoding the IFNlambdaR1 binding molecule or recombinantly modified host cells expressing the IFNlambdaR1 binding molecule) to a subject in need of treatment. The pharmaceutical formulation comprising a IFNlambdaR1 binding molecules of the present disclosure may be administered to a subject in need of treatment or prophyaxis by a variety of routes of administration, including parenteral administration, oral, topical, or inhalation routes.

Parenteral Administration:

In some embodiments, the methods of the present disclosure involve the parenteral administration of a pharmaceutical formulation comprising a IFNlambdaR1 binding molecule (and/or nucleic acids encoding the IFNlambdaR1 binding molecule or recombinantly modified host cells expressing the IFNlambdaR1 binding molecule) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for parenteral administration.

Oral Administration:

In some embodiments, the methods of the present disclosure involve the oral administration of a pharmaceutical formulation comprising a IFNlambdaR1 binding molecule (and/or nucleic acids encoding the IFNlambdaR1 binding molecule or recombinantly modified host cells expressing the IFNlambdaR1 binding molecule) to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

Inhalation Formulations:

In some embodiments, the methods of the present disclosure involve the inhaled administration of a pharmaceutical formulation comprising a IFNlambdaR1 binding molecule (and/or nucleic acids encoding the IFNlambdaR1 binding molecule or recombinantly modified host cells expressing the IFNlambdaR1 binding molecule) to a subject in need of treatment. In the event of administration by inhalation, subject IFNlambdaR1 binding molecules, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

Mucosal and Transdermal Formulations:

In some embodiments, the methods of the present disclosure involve the mucosal or transdermal administration of a pharmaceutical formulation comprising a IFNlambdaR1 binding molecule (and/or nucleic acids encoding the IFNlambdaR1 binding molecule or recombinantly modified host cells expressing the IFNlambdaR1 binding molecule) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

Extended Release and Depot Formulations:

In some embodiments of the method of the present disclosure, the IFNlambdaR1 binding molecule is administered to a subject in need of treatment in a formulation to provide extended release of the IFNlambdaR1 binding molecule agent. Examples of extended release formulations of the injectable compositions can be brought about by including in the composition an agent which delays absorption, for example, aluminum monostearate and gelatin. In one embodiment, the subject IFNlambdaR1 binding molecules or nucleic acids are prepared with carriers that will protect the IFNlambdaR1 binding molecules against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Administration of Nucleic Acids Encoding the IFNlambdaR1 Binding Molecule:

In some embodiments of the method of the present disclosure, delivery of the the IFNlambdaR1 binding molecule to a subject in need of treatment is achieved by the administration of a nucleic acid encoding the IFNlambdaR1 binding molecule. Methods for the administration nucleic acid encoding the IFNlambdaR1 binding molecule to a subject is achieved by transfection or infection using methods known in the art, including but not limited to the methods described in McCaffrey et al. (Nature (2002) 418:6893), Xia et al. (Nature Biotechnol. (2002) 20:1006-1010), or Putnam (Am. J. Health Syst. Pharm. (1996) 53: 151-160 erratum at Am. J. Health Syst. Pharm. (1996) 53:325). In some embodiments, the IFNlambdaR1 binding molecule is administered to a subject by the administration of a pharmaceutically acceptable formulation of recombinant expression vector comprising a nucleic acid sequence encoding the IFNlambdaR1 binding molecule operably linked to one or more expression control sequences operable in a mammalian subject. In some embodiments, the expression control sequence may be selected that is operable in a limited range of cell types (or single cell type) to facilitate the selective expression of the IFNlambdaR1 binding molecule in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the El region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in the target cell type.

In some embodiments, particularly for administration of IFNlambdaR1 binding molecules to the subject, particular for treatment of diseases of the intestinal tract or bacterial infections in a subject, the nucleic acid encoding the IFNlambdaR1 binding molecule may be delivered to the subject by the administration of a recombinantly modified bacteriophage vector encoding the IFNlambdaR1 binding molecule. As used herein, the terms ‘procaryotic virus,” “bacteriophage” and “phage” are used interchangeably hereinto describe any of a variety of bacterial viruses that infect and replicate within a bacterium. Bacteriophage selectively infect procaryotic cells, restricting the expression of the IFNlambdaR1 binding molecule to procaryotic cells in the subject while avoiding expression in mammalian cells. A wide variety of bacteriophages capable of selection a broad range of bacterial cells have been identified and characterized extensively in the scientific literature. In some embodiments, the phage is modified to remove adjacent motifs (PAM). Elimination of the of Cas9 sequences from the phage genome reduces ability of the Cas9 endonuclease of the target procaryotic cell to neutralize the invading phage encoding the IFNlambdaR1 binding molecule.

Administration of Recombinantly Modified Cells Expressing the IFNlambdaR1 Binding Molecule:

In some embodiments of the method of the present disclosure, delivery of the the IFNlambdaR1 binding molecule to a subject in need of treatment is achieved by the administration of recombinant host cells modified to express the IFNlambdaR1 binding molecule may be administered in the therapeutic and prophylactic applications described herein. In some embodiments, the recombinant host cells are mammalian cells, e.g., human cells.

In some embodiments, the nucleic acid sequence encoding the IFNlambdaR1 binding molecule (or vectors comprising same) may be maintained extrachromosomally in the recombinantly modified host cell for administration. In other embodiments, the nucleic acid sequence encoding the IFNlambdaR1 binding molecule may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23(3):967-973, a TALEN-nuclease, a Cas9 endonuclease from CRISPR system as or a modified restriction endonuclease to extended sequence specificity (Eisenschmidt, et al. 2005; 33(22): 7039-7047).

In some embodiments, particularly for administration of IFNlambdaR1 binding molecules to the intestinal tract, the IFNlambdaR1 binding molecule may be delivered to the subject by a recombinantly modified procaryotic cell (e.g., Lactobacillus lacti). The use of engineered procaryotic cells for the delivery of recombinant proteins to the intestinal tract are known in the art. See, e.g. Lin, et al. (2017) Microb Cell Fact 16:148. In some embodiments, the engineered bacterial cell expressing the IFNlambdaR1 binding molecule may be administered orally, typically in aqueous suspension, or rectally (e.g. enema).

Therapeutic Applications

The present disclosure further provides methods of treating a subject suffering from a disease disorder or condition by the administration of a therapeutically effective amount of an IFNlambdaR1 binding molecule (or nucleic acid encoding an IFNlambdaR1 binding molecule including recombinant viruses encoding the IFNlambdaR1 binding molecule) of the present disclosure.

### Use In Combination With Supplementary Agents:

In some embodiments of the therapeutic uses of the compositions of the present disclosure, the administration of a therapeutically effective amount of an IFNlambdaR1 binding molecule (or nucleic acid encoding an IFNlambdaR1 binding molecule including recombinant viruses encoding the IFNlambdaR1 binding molecule) are administered in combination with one or more additional active agents (“supplementary agents”).

As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g., IFNlambdaR1 binding molecule) is considered to be administered in combination with a second agent (e.g. a therapeutic autoimmune antibody such as Humira®) if the biological effect resulting from the administration of the first agent persists in the subject at the time of administration of the second agent such that the therapeutic effects of the first agent and second agent overlap. For example, the therapeutic antibodies are sometimes administered by IV infusion every two weeks while the IFNlambdaR1 binding molecules of the present disclosure may be administered more frequently, e.g. daily, BID, or weekly. However, the administration of the first agent (e.g. entaercept) provides a therapeutic effect over an extended time and the administration of the second agent (e.g. an IFNlambdaR1 binding molecule) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the IFNlambdaR1 binding molecule and the supplementary agent(s) are administered or applied sequentially, e.g., where one agent is administered prior to one or more other agents. In other embodiments, the IFNlambdaR1 binding molecule and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

Supplementary agents may administered or introduced separately, for example, formulated separately for separate administration (e.g., as may be provided in a kit) and/or therapies that can be administered or introduced in combination with the IFNlambdaR1 binding molecules.

### Inflammatory and Autoimmune Disorders

Disorders amenable to treatment with IFNλR binding molecules (including pharmaceutically acceptable formulations comprising IFNλR binding molecules and/or the nucleic acid molecules that encode them including recombinant viruses encoding such IFNλR binding molecules) of the present disclosure include inflammatory or autoimmune diseases including but not limited to, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome (Seronegativity, Enthesopathy, Arthropathy Syndrome).

Other examples of proliferative and/or differentiative disorders amenable to treatment with IFNλR binding molecules (including pharmaceutically acceptable formulations comprising IFNλR binding molecules and/or the nucleic acid molecules that encode them including recombinant viruses encoding such IFNλR binding molecules) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of inflammatory or autoimmune skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising IFNλR binding molecules and/or the nucleic acid molecules that encode them including recombinant viruses encoding such IFNλR binding molecules) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

Supplemental Agents Useful in the Treatment of Inflammatory or Autoimmune Disorders

In some embodiments, the method further comprises administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody. In certain embodiments, the therapeutic antibody is an antibody that binds a protein selected from the group consisting of BLyS, CD11a, CD20, CD25, CD3, CD52, IgEIL12/IL23, IL17a, IL1β, IL4Rα, IL5, IL6R, integrin-α4β7, RANKL, TNFα, VEGF-A, and VLA-4.

In some embodiments, the supplementary agent is one or more agents selected from the group consisting of corticosteroids (including but not limited to prednisone, budesonide, prednisone), Janus kinase inhibitors (including but not limited to tofacitinib (Xeljanz®), calcineurin inhibitors (including but not limited to cyclosporine and tacrolimus), mTor inhibitors (including but not limited to sirolimus and everolimus), IMDH inhibitors (including but not limited to azathioprine, leflunomide and mycophenolate), biologics such as abatcept (Orencia®) or etanercept (Enbrel®), and therapeutic antibodies.

Examples of therapeutic antibodies that may be administered as supplementary agents in combination with the IFNλR binding molecules of the present disclosure in the treatment of autoimmune disease include but are not limited to anti-CD25 antibodies (e.g. daclizumab and basiliximab), anti-VLA-4 antibodies (e.g. natalizumab), anti-CD52 antibodies (e.g. alemtuzumab), anti-CD20 antibodies (e.g. rituximab, ocrelizumab), anti-TNF antibodies (e.g. infliximab, and adalimumab), anti-IL6R antibodies (e.g. tocilizumab), anti-TNFα antibodies (e.g. adalimumab (Humira®), golimumab, and infliximab), anti-integrin-α4β7 antibodies (e.g. vedolizumab), anti-IL17a antibodies (e.g. brodalumab or secukinumab), anti-IL4Rα antibodies (e.g. dupilumab), anti-RANKL antibodies, IL6R antibodies, anti-IL1B antibodies (e.g. canakinumab), anti-CD11a antibodies (e.g. efalizumab), anti-CD3 antibodies (e.g. muramonab), anti-IL5 antibodies (e.g. mepolizumab, reslizumab), anti-BLyS antibodies (e.g. belimumab); and anti-IL12/IL23 antibodies (e.g ustekinumab).

Many therapeutic antibodies have been approved for clinical use against autoimmune disease. Examples of antibodies approved by the United States Food and Drug Administration (FDA) for use in the treatment of autoimmune diseases in a subject suffering therefrom that may be administered as supplementary agents in combination with the IFNλR binding molecules of the present disclosure (and optionally additional supplementary agents) for the treatment of the indicated autoimmune disease are provided in Table X below.

TABLE X
Supplementary Agents for the Treatment of Autoimmune and Inflammatory Disease

| Name | Target | Indication |
|---|---|---|
| belimumab | BLyS | Systemic lupus erythematosus |
| efalizumab | CD11a | Psoriasis |
| ocrelizumab | CD20 | Multiple sclerosis |
| rituximab | CD20 | Multiple sclerosis |
| basiliximab | CD25 | Transplantation rejection |
| daclizumab | CD25 | Transplantation rejection |
| muromonab | CD3 | Transplantation rejection |
| alemtuzumab | CD52 | Multiple sclerosis |
| omalizumab | IgE | Asthma |
| ustekinumab | IL12/IL23 | Plaque psoriasis |
| brodalumab | IL17a | Psoriasis, psoriatic arthritis, ankylosing spondylitis |
| secukinumab | IL17a | Psoriasis, psoriatic arthritis, ankylosing spondylitis |
| ixekizumab | IL17a | Psoriasis, psoriatic arthritis, ankylosing spondylitis |
| canakinumab | IL1β | Cryopyrin-associated periodic syndrome, tumor necrosis factor receptor |
| | | associated periodic syndrome, hyperimmunoglobulin D syndrome, |
| | | mevalonate kinase deficiency, familial Mediterranean fever, rheumatoid |
| | | arthritis |
| dupilumab | IL4Rα | Asthma, dermatitis |
| mepolizumab | IL5 | Asthma |
| reslizumab | IL5 | Asthma |
| tocilizumab | IL6R | Rheumatoid arthritis |
| vedolizumab | Integrin-α4β7 | Ulcerative colitis, Crohn's disease |
| denosumab | RANKL | Osteoporosis |
| certolizumab | TNFa | Chron's disease, rheumatoid arthritis |
| golimumab | TNFa | Rheumatoid arthritis, psoriatic arthritis, ankylosing spondylitis |
| adalimumab | TNFα | Rheumatoid arthritis, juvenile idiopathic arthritis, psoriatic arthritis, |
| | | ankylosing spondylitis, Crohn's disease, plaque psoriasis |
| infliximab | TNFα | Crohn's disease, ulcerative colitis, rheumatoid arthritis, ankylosing |
| | | spondylitis, psoriatic arthritis, plaque psoriasis |
| ranibizumab | VEGF-A | Neovascular age-related macular degeneration, macular edema |
| natalizumab | VLA-4 | Multiple sclerosis, relapsing rultiple sclerosis, Crohn's disease |

The foregoing antibodies useful as supplementary agents in the practice of the methods of the present disclosure may be administered alone or in the form of any antibody drug conjugate (ADC) comprising the antibody, linker, and one or more drugs (e.g. 1, 2, 3, 4, 5, 6, 7, or 8 drugs) or in modified form (e.g. PEGylated).

Prophylactic Applications

In some embodiments where the IFNlambdaR1 binding molecule is used in prophylaxis of disease, the supplementary agent may be a vaccine. The IFNlambdaR1 binding molecule of the present invention may be administered to a subject in combination with vaccines as an adjuvant to enhance the immune response to the vaccine in accordance with the teaching of Doyle, et al U.S. Pat. No. 5,800,819 issued Sep. 1, 1998. Examples of vaccines that may be combined with the IFNlambdaR1 binding molecule of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

### EXAMPLES

### Example 1—V_{H}H Generation

Camels were acclimated at research facility for at least 7 days before immunization. Antigen was diluted with 1×PBS (antigen total about 1 mg). The quality of the antigen was assessed by SDS-PAGE to ensure purity (e.g., >80%). For the first time, 10 mL CFA (then followed 6 times using IFA) was added into mortar, then 10 mL antigen in 1×PBS was slowly added into the mortar with the pestle grinding. The antigen and CFA/IFA were ground until the component showed milky white color and appeared hard to disperse. Camels were injected with antigen emulsified in CFA subcutaneously at at least six sites on the body, injecting about 2 mL at each site (total of 10 mL per camel). A stronger immune response was generated by injecting more sites and in larger volumes. The immunization was conducted every week (7 days), for 7 times. The needle was inserted into the subcutaneous space for 10 to 15 seconds after each injection to avoid leakage of the emulsion. Alternatively, a light pull on the syringe plunger also prevented leakage. The blood sample was collected three days later after 7th immunization.

After immunization, the library was constructed. Briefly, RNA was extracted from blood and transcribed to cDNA. The VHH regions were obtained via two-step PCR, which fragment about 400 bp. The PCR outcomes and the vector of pMECS phagemid were digested with Pst I and Not I, subsequently, ligated to pMECS/Nb recombinant. After ligation, the products were transformed into Escherichia coli (E. coli) TG1 cells by electroporation. Then, the transformants were enriched in growth medium and planted on plates. Finally, the library size was estimated by counting the number of colonies.

Bio-panning of the phage library was conducted to identify VHHs that bind. A 96-well plate was coated with antigen and the phage library was incubated in each well to allow phage-expressing antigen reactive VHH to bind to the antigen on the plate. Non-specifically bound phage were washed off and the specifically bound phage isolated. After the selection, the enriched phage library expressing antigen reactive VHH were amplified in TG1 cells. The aforementioned bio-panning process was repeated for 2-3 rounds to enrich the library for VHH selective for antigen. Once biopanning was complete, three 96-well plates of individual phage clones were isolated in order to perform periplasmic extract ELISA PE-ELISA) on antigen coated plates to identify positive VHH binders. Briefly, A 96-well plate was coated with antigen and PBS under the same conditions. Next, wells were blocked at 37° C. for 1 h. Then, 100 μl of extracted antibodies was added to each well and incubated for 1 h. Subsequently, 100 μl of anti-tag polyclonal antibody conjugated to HRP was added to each well and incubated at 37° C. for 1 h. Plates were developed with TMB substrate. The reaction was stopped by the addition of H2SO4. Absorbance at 450 nm was read on a microtiter plate reader and antibodies with absorbance of the antigen-coated well at least threefold greater than PBS-coated control were considered positive binding molecules and subjected to sequence analysis.

### Example 2—Recombinant Production and Purification

Codon optimized DNA inserts were cloned into modified pcDNA3.4 (Genscript) for small scale expression in HEK293 cells in 24 well plates. The binding molecules were purified in substantial accordance with the following procedure. Using a Hamilton Star automated system, 96×4 mL of supernatants in 4×24-well blocks were re-arrayed into 4×96-well, 1 mL blocks. PhyNexus micropipette tips (Biotage, San Jose CA) holding 80 μL of Ni-Excel IMAC resin (Cytiva) are equilibrated wash buffer: PBS pH 7.4, 30 mM imidazole. PhyNexus tips were dipped and cycled through 14 cycles of 1 mL pipetting across all 4×96-well blocks. PhyNexus tips were washed in 2×1 mL blocks holding wash buffer. PhyNexus tips were eluted in 3×0.36 mL blocks holding elution buffer: PBS pH 7.4, 400 mM imidazole. PhyNexus tips were regenerated in 3×1 mL blocks of 0.5 M sodium hydroxide.

The purified protein eluates were quantified using a Biacore® T200 as in substantial accordance with the following procedure. 10 uL of the first 96×0.36 mL eluates were transferred to a Biacore® 96-well microplate and diluted to 60 uL in HBS-EP+buffer (10 mM Hepes pH 7.4, 150 mM NaCl, 1 mM EDTA, 0.05% Tween 20). Each of the 96 samples was injected on a CM5 series S chip previously functionalized with anti-histidine capture antibody (Cytiva): injection is performed for 18 seconds at 5 μL/min. Capture levels were recorded 60 seconds after buffer wash. A standard curve of known VHH concentrations (270, 90, 30, 10, 3.3, 1.1 μg/mL) was acquired in each of the 4 Biacore chip flow cells to eliminate cell-to-cell surface variability. The 96 captures were interpolated against the standard curve using a non-linear model including specific and unspecific, one-site binding. Concentrations in the first elution block varied from 12 to 452 μg/mL corresponding to a 4-149 μg. SDS-PAGE analysis of 5 randomly picked samples was performed to ensure molecular weight of eluates corresponded to expected values (˜30 kDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent. The final, normalized plate was sterile filtered using 0.22 μm filter plates (Corning).

The IL10Rb binding molecules provided in Tables 5 and 6 generated in accordance with the teaching of present disclosure exhibit specific binding and provided a range of affinities to the extracellular domain of IL10Rb. The IL28RA binding molecules provided in Table 7 generated in accordance with the teaching of present disclosure exhibit specific binding and provided a range of affinities to the extracellular domain of IL28RA.

It is understood that the embodiments described herein are for illustrative purposes only and that various modifications or changes in light thereof will be suggested to persons skilled in the art and are to be included within the spirit and purview of this application and scope of the appended claims. All publications, patents, patent applications, and the sequences of the sequence accession numbers cited herein are hereby incorporated by reference.

### Tables

TABLE 2
anti-hIL 10Rb sdAb CDRs

| | | SEQ ID | | SEQ ID | | SEQ ID |
|---|---|---|---|---|---|---|
| Name | CDR1 | NO: | CDR2 | NO: | CDR3 | NO: |
| hIL10Rb_ | YTYSSGCMG | 25. | AINSDGSTSYADSVKG | 52. | EPYCSGGYPRWS | 79. |
| VHH-1 | | | | | VAEFGY | |
| hIL10Rb_ | YTYSSYCMG | 26. | AIDSDGSTRYADSVKG | 53. | EPYCSGGYKRTM | 80. |
| VHH-2 | | | | | VAEFGY | |
| hIL10Rb_ | YTYNSYCMG | 27. | TIDSDGMTRYADSVKG | 54. | DADCTIAAMTTN | 81. |
| VHH-3 | | | | | P | |
| hIL10Rb_ | YLYSIDYMA | 28. | VIYTASGATFYPDSVKG | 55. | VRKTDSYLFDAQ | 82. |
| VHH-4 | | | | | SFTY | |
| hIL10Rb_ | YTYSSYCMG | 29. | HIDSDGSTTYADSVKG | 56. | DPIPGPGYCDGG | 83. |
| VHH-5 | | | | | PNKY | |
| hIL10Rb_ | DLYSTNYVA | 30. | VIYTASGATLYSDSVKG | 57. | VRKTGHYLFDAQ | 84. |
| VHH-6 | | | | | SFTY | |
| hIL10Rb_ | YTYSSGCMG | 31. | TINSDGSTNYADSVKG | 58. | EPYCSGGYPRWS | 85. |
| VHH-7 | | | | | VAEFGY | |
| hIL10Rb_ | YSYSSYCMG | 32. | AIASDGSTSYADSVKG | 59. | EPWCTGGYSRLT | 86. |
| VHH-8 | | | | | PAEYGY | |
| hIL10Rb_ | YTYSSGCMG | 33. | TINSDGSTNYADSVKG | 60. | EPYCSGGYPRWS | 87. |
| VHH-9 | | | | | VAEFGY | |
| hIL10Rb_ | YTYSSYCMG | 34. | HIDSDGSTTYADSVKG | 61. | DPIPGPGYCDGG | 88. |
| VHH-10 | | | | | PNKY | |
| hTL10Rb | YTYSSYCMG | 35. | AIDSDGSTRYADSVKG | 62. | EPYCSGGYKRTM | 89. |
| VHH-11 | | | | | VAEFGY | |
| hIL10Rb_ | YTYSSYCMG | 36. | HIDSDGSTSYADSVKG | 63 | DPIPGPGYCDGG | 90. |
| VHH-12 | | | | | PNKY | |
| hIL10Rb_ | YTYSSYCMG | 37. | HIDSDGSTSYADSVKG | 64 | DPIPGPGYCDGG | 91. |
| VHH-13 | | | | | PNNY | |
| hIL10Rb_ | YTYSSGCMG | 38. | TINSDGSTNYADSVKG | 65. | EPYCSGGYPRWS | 92. |
| VHH-14 | | | | | VAEFGY | |
| hIL10Rb_ | YTASVNYMG | 39. | TIFTGAGTTYYANSVKG | 66. | DFRGGLLYRPAY | 93. |
| VHH-15 | | | | | EYTY | |
| hIL10Rb_ | YTHSSYCMG | 40. | AIDVDGSTTYADSVKG | 67. | EFADCSSNYFLP | 94. |
| VHH-16 | | | | | PGAVRY | |
| hIL10Rb_ | YTASVNYMG | 41. | TIFTGAGTTYYANSVKG | 68. | DFRGGLLYRPAY | 95. |
| VHH-17 | | | | | EYTY | |
| hIL10Rb_ | DTYSSYCMG | 42. | FIDSDGSTRYADSVEG | 69. | EPYCSGGYHRKE | 96. |
| VHH-18 | | | | | MAEFGY | |
| hIL10Rb_ | YTYSSYCMG | 43. | HIDSDGSTSYADSVKG | 70. | DPIPGPGYCDGG | 97. |
| VHH-19 | | | | | PNKY | |
| hIL10Rb_ | YTYSSYCMG | 44. | HIDSDGSTTYADSVKG | 71. | DPIPGPGYCDGG | 98. |
| VHH-20 | | | | | PNKY | |
| hIL10Rb_ | YTASNNCMG | 45. | VIFTGAGTSYYDSSVG | 72. | EDDCTLLLMTPN | 99. |
| VHH-21 | | | | | PDDQ | |
| hIL10Rb_ | YTDSRYCMG | 46. | HIDSDGSTSYADSVKG | 73. | DPIPGPGYCDGG | 100. |
| VHH-22 | | | | | PNKY | |
| hIL10Rb_ | YTYSSYCMG | 47. | AIDSDGSTRYADSVKG | 74. | EPYCSGGYKRTM | 101. |
| VHH-23 | | | | | VAEFGF | |
| hIL10Rb_ | YTYSSYCMG | 48. | HIDSDGSTTYADSVKG | 75. | DPIPGPGYCDGG | 102. |
| VHH-24 | | | | | PNNY | |
| hIL10Rb_ | YTYSSYCMG | 49. | HIDSDGSTTYADSVKG | 76. | DPIPGPGYCDGG | 103. |
| VHH-25 | | | | | PNNY | |
| hIL10Rb_ | YSYSSYCMG | 50. | TIDSDGMTRYADSVKG | 77. | PLYDCDSGAVGR | 104. |
| VHH-26 | | | | | NPPY | |
| hIL10Rb_ | YTYLRGCMG | 51. | VMDVVGDRRSYIDSVKG | 78. | GPNCVGWRSGLD | 105. |
| VHH-27 | | | | | Y | |

TABLE 3
anti-mIL 10Rb sdAb CDRs

| | | SEQ ID | | SEQ ID | | SEQ ID |
|---|---|---|---|---|---|---|
| Name | CDR1 | NO: | CDR2 | NO: | CDR3 | NO: |
| DR1322 | YTASSICMG | 106. | VITTAASGTY | 112. | TRRGGDCLDPLQT | 118. |
| | | | YADSVNG | | PAYNT | |
| DR1323 | DTYSRKYIA | 107. | VMYTPGSATY | 113. | KASGSMENFRDYT | 119. |
| | | | YTDTVMG | | Y | |
| DR1324 | YASCSRAMR | 108. | YIDGVGSTGY | 114. | GCRADGSNSLDNY | 120. |
| | | | ADSVKG | | | |
| DR1325 | YTYNRREMG | 109. | IIYTPNSSTF | 115. | ARIASMTELSVRD | 121. |
| | | | YADSVTG | | MDY | |
| DR1326 | YIALNACMA | 110. | TIVTDGSRTY | 116. | DRRCPVSRAPYEY | 122. |
| | | | YADSVKG | | ELRY | |
| DR1327 | YTYNGKCMA | 111. | GIYTGGSSTY | 117. | SRSCSDLRRRSIA | 123. |
| | | | YADSVKG | | Y | |

TABLE 4
anti-hIL28RA sdAb CDRs

| | | SEQ ID | | SEQ ID | | SEQ ID |
|---|---|---|---|---|---|---|
| Name | CDR1 | NO: | CDR2 | NO: | CDR3 | NO: |
| hIL28R_ | YISSSYCMA | 124. | GVTRDGKTYYG | 138. | GPPPCITSMPAGGDYG | 152. |
| VHH1 | | | DSVKG | | YRY | |
| hIL28R_ | FTFSNYGMS | 125. | GINSGGDDTFY | 139. | GASGMIP | 153. |
| VHH2 | | | TDSVKG | | | |
| hIL28R_ | FTFSDYAMS | 126. | AIGRDGSTFYP | 140. | EEPGSSS | 154. |
| VHH3 | | | DSVKG | | | |
| hIL28R_ | STDNIKYMG | 127. | AVYTSGGAVVY | 141. | SRAPAPPRLLLQRALV | 155. |
| VHH4 | | | ADSVKG | | EY | |
| hIL28R_ | FTFSNATMS | 128. | AISNSRGTKYY | 142. | DWKTSYSDYDLS | 156. |
| VHH5 | | | AAFVKG | | | |
| hIL28R_ | FTFSDYAMS | 129. | AIGRDGSTFYP | 143. | EEPGSSS | 157. |
| VHH6 | | | DSVKG | | | |
| hIL28R_ | FTFSNYGMS | 130. | GINSGGDDTFY | 144. | GASGMIP | 158. |
| VHH7 | | | TDSVKG | | | |
| hIL28R_ | YTISRSDCMG | 131. | RIGSDGTTSYA | 145. | TALLLGRGSACHKEVS | 159. |
| VHH8 | | | DSVKE | | VFSW | |
| hIL28R_ | FTFSNYGMS | 132. | GINSGGDDTFY | 146. | GASGMIP | 160. |
| VHH9 | | | TDSVKG | | | |
| hIL28R_ | YISSRSTYCM | 133. | VVTGDSRTYYG | 147. | GPPPCITTMPAGGDYG | 161. |
| VHH10 | G | | DSVKG | | YRY | |
| hIL28R_ | FTYSSYCMG | 134. | AIDSDGSTSYA | 148. | DGEYNDYVCWSTGLRY | 162. |
| VHH11 | | | DSVKG | | | |
| hIL28R_ | YISSRSTYCM | 135. | IVTGDSRTYYG | 149. | GPPPCITSMPAGGDYG | 163. |
| VHH12 | G | | DSVRG | | YRY | |
| hIL28R_ | FTFSNATMS | 136. | AISNSRGTKYY | 150. | DWKTSYSDYDLS | 164. |
| VHH13 | | | AAFVKG | | | |
| hIL28R_ | YISRSSYCMG | 137. | IVTGDGRTYYG | 151. | GPPPCITTMPAGGDYG | 165. |
| VHH14 | | | DSVKG | | YRY | |

TABLE 5
anti-hIL10Rb sdAb VHH AMINO ACID SEQUENCE

| Name | Sequence | SEQ ID NO: |
|---|---|---|
| hIL10Rb_VHH-1 | QVQLQESGGGSVQAGGSLRLSCAASGYTYSSGCMGWFRQAPGKEREAVAAIN | 166. |
| | SDGSTSYADSVKGRFTISKDNAKNTLYLQMNSLKPEDTAMYYCAAEPYCSGG | |
| | YPRWSVAEFGYWGQGTQVTVSS | |
| hIL10Rb_VHH-2 | QVQLQESGGGSVQAGGSLRLSCAASGYTYSSYCMGWFRQAPGKEREGVAAID | 167. |
| | SDGSTRYADSVKGRFTISKDNAKNTLYLQMNSLKPEDTAMYYCAAEPYCSGG | |
| | YKRTMVAEFGYWGQGTQVTVSS | |
| hIL10Rb_VHH-3 | QVQLQESGGGSVQAGGSLRLSCAASRYTYNSYCMGWFRQAPGKEREGVATID | 168. |
| | SDGMTRYADSVKGRFTISKDNAKNTLYLQMNSLKPEDTAMYYCAADADCTIA | |
| | AMTTNPLGQGTQVTVSS | |
| hIL10Rb_VHH-4 | QVQLQESGGGSIQAGGSLRLSCAASRYLYSIDYMAWFRQSPGKEREPVAVIY | 169. |
| | TASGATFYPDSVKGRFTISQDNAKMTVYLQMNSLKSEDTAMYYCAAVRKTDS | |
| | YLFDAQSFTYWGQGTQVTVSS | |
| hIL10Rb_VHH-5 | QVQLQESGGGLVQPGGSLRLSCAASGYTYSSYCMGWFRQAPGKEREGVAHID | 170. |
| | SDGSTTYADSVKGRFTISKDNAKNTLYLQMNSLKPEDTAMYYCAADPIPGPG | |
| | YCDGGPNKYWGQGTQVTVSS | |
| hIL10Rb_VHH-6 | QVQLQESGGGSIQAGGSLTLSCAASRDLYSTNYVAWFRQSPGKEREAVAVIY | 171. |
| | TASGATLYSDSVKGRFTISQDNAKMTVYLQMNSLKSEDTAMYYCAAVRKTGH | |
| | YLFDAQSFTYWGQGTQVTVSS | |
| hIL10Rb_VHH-7 | QVQLQESGGGSVQAGGSLRLSCAASGYTYSSGCMGWFRQAPGKEREGVATIN | 172. |
| | SDGSTNYADSVKGRFTISKDNAKNTLYLQMNSLKPEDTAMYYCAAEPYCSGG | |
| | YPRWSVAEFGYWGQGTQVTVSS | |
| hIL10Rb_VHH-8 | QVQLQESGGGSVQAGGSLRLSCAASGYSYSSYCMGWFRQAPGKEREGVAAIA | 173. |
| | SDGSTSYADSVKGRFAISKDNAKNTLYLQMASLKPEDTAMYYCAAEPWCTGG | |
| | YSRLTPAEYGYWGQGTQVTVSS | |
| hIL10Rb_VHH-9 | QVQLQESGGGLVQPGGSLRLSCAASGYTYSSGCMGWFRQAPGKEREGVATIN | 174. |
| | SDGSTNYADSVKGRFTISKDNAKNTLYLQMNSLKPEDTAMYYCAAEPYCSGG | |
| | YPRWSVAEFGYWGQGTQVTVSS | |
| hIL10Rb_VHH-10 | QVQLQESGGGLVQPGGSLRLSCAASGYTYSSYCMGWFRQAPGKEREGVAHID | 175. |
| | SDGSTTYADSVKGRFAISKDNAKNTLYLQMNSLKPEDTAMYYCAADPIPGPG | |
| | YCDGGPNKYWGQGTQVTVSS | |
| hIL10Rb_VHH-11 | QVQLQESGGGSVQAGGSLRLSCAASGYTYSSYCMGWFRQAPGKGREGVAAID | 176. |
| | SDGSTRYADSVKGRFTISKDNAKNTLYLQMNSLKPEDTAMYYCAAEPYCSGG | |
| | YKRTMVAEFGYWGQGTQVTVSS | |
| hIL10Rb_VHH-12 | QVQLQESGGGSVQAGGSLRLSCAASGYTYSSYCMGWFRQAPGKEREGVAHID | 177. |
| | SDGSTSYADSVKGRFTISKDNAKNTLYLQMNSLKPEDTAMYYCAADPIPGPG | |
| | YCDGGPNKYWGQGTQVTVSS | |
| hIL10Rb_VHH-13 | QVQLQESGGGSVQAGGSLRLSCAASGYTYSSYCMGWFRQAPGKEREGVAHID | 178. |
| | SDGSTSYADSVKGRFTISKDNAKNTLYLQMNSLKPEDTAMYYCAADPIPGPG | |
| | YCDGGPNNYWGQGTQVTVSS) | |
| hIL10Rb_VHH-14 | QVQLQESGGGSVQAGGSLRLSCAASGYTYSSGCMGWFRQAPGKEREGVATIN | 179. |
| | SDGSTNYADSVKGRFTISKDNAKNTLYLQMNSLKPEDTGMYYCAAEPYCSGG | |
| | YPRWSVAEFGYWGQGTQVTVSS | |
| hIL10Rb_VHH-15 | QVQLQESGGGSVQAGGSLRLSCTVSRYTASVNYMGWFRQAPGKEREGVATIF | 180. |
| | TGAGTTYYANSVKGRFTISRDNAKNTAYLQMNSLKPEDTAIYYCAVDFRGGL | |
| | LYRPAYEYTYRGQGTQVTVSS | |
| hIL10Rb_VHH-16 | QVQLQESGGGSVEAGGSLRLSCAASGYTHSSYCMGWFRQAPGKEREGVAAID | 181. |
| | VDGSTTYADSVKGRFTISKDNAKNTLYLQMNSLKPEDTGMYYCAAEFADCSS | |
| | NYFLPPGAVRYWGQGTQVTVSS | |
| hIL10Rb_VHH-17 | QVQLQESGGGSVQAGGSLRLSCTVSRYTASVNYMGWFRQAPGKEREGVATIF | 182. |
| | TGAGTTYYANSVKGRFTISRDNAKNTAYLQMNSLKPEDTAMYYCAVDFRGGL | |
| | LYRPAYEYTYRGQGTQVTVSS | |
| hIL10Rb_VHH-18 | QVQLQESGGGSVQAGGSLRLSCAASGDTYSSYCMGWFRQAPGKEREGVAFID | 183. |
| | SDGSTRYADSVEGRFTISKDNAKNTLYLQMNSLKPEDTAMYYCAAEPYCSGG | |
| | YHRKEMAEFGYWGQGTQVTVSS | |
| hIL10Rb_VHH-19 | QVQLQESGGGSVQAGGSLRLSCAASGYTYSSYCMGWFRQAPGKEREGVAHID | 184. |
| | SDGSTSYADSVKGRFTISKDNAKNTLYLQMNSLKPEDTAMYYCAADPIPGPG | |
| | YCDGGPNKYRGQGTQVTVSS | |
| hIL10Rb_VHH-20 | QVQLQESGGGSVQAGGSLRLSCAASGYTYSSYCMGWFRQAPGKEREGVAHID | 185. |
| | SDGSTTYADSVKGRFAISKDNAKNTLYLQMNSLKPEDTAMYYCAADPIPGPG | |
| | YCDGGPNKYWGQGTQVTVSS | |
| hIL10Rb_VHH-21 | QVQLQESGGGSVQAGGSLRLSCTGSGYTASNNCMGWFRQAPGKEREGVAVIF | 186. |
| | TGAGTSYYDSSVGRLFISSQDAASTLDQLLMSLLPDDTAVMYCGAEDDCTLL | |
| | LMTPNPDDQWSRLSVSS | |
| hIL10Rb_VHH-22 | QVQLQESGGGSVQAGGSLRLSCAASGYTDSRYCMGWFRKAPGKEREGVAHID | 187. |
| | SDGSTSYADSVKGRFTISKDNAKNTLYLQMNSLKPEDTAMYYCAADPIPGPG | |
| | YCDGGPNKYWGQGTQVTVSS | |
| hIL10Rb_VHH-23 | QVQLQESGGGSVQAGGSLRLSCAASGYTYSSYCMGWFRQAPGKEREGVAAID | 188. |
| | SDGSTRYADSVKGRFTISKDNAKKILYLQMNSLKVEDTAMYYCAAEPYCSGG | |
| | YKRTMVAEFGFWGQGTQVTVSS | |
| hIL10Rb_VHH-24 | QVQLQESGGGSVQAGGSLKLSCAASGYTYSSYCMGWFRQAPGKEREGVAHID | 189. |
| | SDGSTTYADSVKGRFTISKDNAKNTLYLQMNSLKPEDTAMYYCAADPIPGPG | |
| | YCDGGPNNYWGQGTQVTVSS | |
| hIL10Rb_VHH-25 | QVQLQESGGGSVQAGGSLRLSCAASGYTYSSYCMGWFRQAPGKEREGIAHID | 190. |
| | SDGSTTYADSVKGRFTISKDNAKNTLYLQMNSLKPEDTAMYYCAADPIPGPG | |
| | YCDGGPNNYWGQGTQVTVSS | |
| hIL10Rb_VHH-26 | QVQLQESGGGSVQAGGSLRLSCAASGYSYSSYCMGWFRQAPGKEREGVATID | 191. |
| | SDGMTRYADSVKGRFTISKDNAKNTLYLQMNSLKPEDTAMYYCAAPLYDCDS | |
| | GAVGRNPPYWGQGTQVTVSS | |
| hIL10Rb_VHH-27 | QVQLQESGGGSVQTGGSLRLSCAASGYTYLRGCMGWFRQAPGKEREGVAVMD | 192. |
| | VVGDRRSYIDSVKGRFTISRDNAANSVYLQMDNLKPEDTAMYYCTAGPNCVG | |
| | WRSGLDYWGQGTQVTVSS | |

TABLE 6
anti-mIL 10Rb sdAb VHH AMINO ACID SEQUENCE

| | | SEQ ID |
|---|---|---|
| Name | Sequence | NO: |
| DR1322 | QVQLQESGGGSVQAGGALRLSCTASGYTASSICMGWFRQAPGKERERVAVITTAASGTY | 193. |
| | YADSVNGRESISQNNAKNTVYLQMNSLKPDDTAMYYCAATRRGGDCLDPLQTPAYNTWG | |
| | QGTQVTVSS | |
| DR1323 | QVQLQESGGGSVQAGGSLRLSCVASGDTYSRKYIAWVRQVPGKEREGVAVMYTPGSATY | 194. |
| | YTDTVMGRFTISQDNAKNTVYLQMNSLKPEDTAMYFCAAKASGSMENFRDYTYWGQGTQ | |
| | VTVSS | |
| DR1324 | QVQLQESGGGSVQAGGSLRLSCATSGYASCSRAMRWYRQAPGKEREFVAYIDGVGSTGY | 195. |
| | ADSVKGRFTISQDNAKYTAYLQMNSLKPEDTAMYYCNRGCRADGSNSLDNYWGQGTQVT | |
| | VSS | |
| DR1325 | QVQLQESGGGSVQAGGSLRLSCAASGYTYNRREMGWFRQAPGKEREGLAIIYTPNSSTF | 196. |
| | YADSVTGRFTISQDSARNTVYLQMNSLKPEDTAMYYCAAARIASMTELSVRDMDYWGKG | |
| | TQVTVSS | |
| DR1326 | QVQLQESGGGSVQAGGSLRLSCTASRYIALNACMAWIRQAPGSEREVVATIVTDGSRTY | 197. |
| | YADSVKGRFTISQDNAKNTMYLQMNGLKPEDTAMYYCAADRRCPVSRAPYEYELRYWGQ | |
| | GTQVTVSS | |
| DR1327 | QVQLQESGGGSVQAGGSLRLSCAASGYTYNGKCMAWFRQAPGKEREVVAGIYTGGSSTY | 198. |
| | YADSVKGRFTISQDNAKNTVYLQMDSLKPEDTAMYYCATSRSCSDLRRRSIAYWGQGTQ | |
| | VTVSS | |

TABLE 7
anti-hIL28RA sdAb is a VHH AMINO ACID SEQUENCE

| | | SEQ ID |
|---|---|---|
| Name | Sequence | NO: |
| hIL28R_ | QVQLQESGGGSVQAGGSLRLSCASSGYISSSYCMAWFRQAPGKEREGAAGVTRDGKT | 199. |
| VHH1 | YYGDSVKGRFAISRDNAKNTLYLQMNSLKPEDTAMYYCAAGPPPCITSMPAGGDYGY | |
| | RYWGQGTQVTVSS | |
| hIL28R_ | QVQLQESGGGLVQPGGSLRLSCAASGFTFSNYGMSWVRQAPGKGLEWVSGINSGGDD | 200. |
| VHH2 | TFYTDSVKGRFTISRDNAKNTLYLQMNSLKTEDTAMYYCAMGASGMIPRGQGTQITV | |
| | SS | |
| hIL28R_ | QVQLQESGGGLVQPGGSLRLSCVASGFTFSDYAMSWVRQAPGMGLERVSAIGRDGST | 201. |
| VHH3 | FYPDSVKGRFTISRDNAKNTLYLQLNSLKTEDTAMYYCAKEEPGSSSRGQGTQVTVS | |
| | S | |
| hIL28R_ | QVQLQESGGGSVQLGGSLRLSCLVSGSTDNIKYMGWFRQAPGKEREGVAAVYTSGGA | 202. |
| VHH4 | VVYADSVKGRFTISQDDAKNTMYLQMNSLKPEDTAMYYCAASRAPAPPRLLLQRALV | |
| | EYWGQGTQVTVSS | |
| hIL28R_ | QVQLQESGGGLVQPGGSLRLSCAASGFTFSNATMSWVRQAPGKEIEWVSAISNSRGT | 203. |
| VHH5 | KYYAAFVKGRFTISRDNAKNTLYLQLNNLKTEDTAMYYCTKDWKTSYSDYDLSDGQG | |
| | TQVTVSS | |
| hIL28R_ | QVQLQESGGGLVQPGGSLRLSCAASGFTFSDYAMSWVRQAPGMGLERVSAIGRDGST | 204. |
| VHH6 | FYPDSVKGRFTISRDNAKNTLYLQLNSLKTEDTAMYYCAKEEPGSSSRGQGTQVTVS | |
| | S | |
| hIL28R_ | QVQLQESGGGLVQPGGSLRLSCAASGFTFSNYGMSWVRQAPGKGLEWVSGINSGGDD | 205. |
| VHH7 | TFYTDSVKGRFTISRDNAKNTLYLQMNSLKTEDTAMYYCAMGASGMIPRGQGTQVTV | |
| | SS | |
| hIL28R_ | QVQLQESGGGSVQAGGSLRLSCAVSRYTISRSDCMGWFRQAPGKEREGVARIGSDGT | 206. |
| VHH8 | TSYADSVKERFTISKDNAKNILYLQMNSLKPEDTARYYCAATALLLGRGSACHKEVS | |
| | VFSWWGQGTQVTVSS | |
| hIL28R_ | QVQLQESGGGLVQPGGSLRLSCAASGFTFSNYGMSWVRQAPGKGLEWVSGINSGGDD | 207. |
| VHH9 | TFYTDSVKGRFTISRDNVKNTLYLQMNSLKTEDTAMYYCAMGASGMIPRGQGTQVTV | |
| | SS | |
| hIL28R | QVQLQESGGGSVQAGGSLRLSCASSGYISSRSTYCMGWFRQAPGKEREVAAVVTGDS | 208. |
| VHH10_ | RTYYGDSVKGRFAISRDNAKNTLYLQMNSLKPEDTAMYYCAAGPPPCITTMPAGGDY | |
| | GYRYWGQGTQVTVSS | |
| hIL28R_ | QVQLQESGGGSVQSGGSLRLSCAASGFTYSSYCMGWFRQAPGKEREGVAAIDSDGST | 209. |
| VHH11 | SYADSVKGRFTISKDNAKNTLYLQMNSLRPEDTAMYYCAADGEYNDYVCWSTGLRYR | |
| | GQGTQVTVSS | |
| hIL28R_ | QVQLQESGGGSVQAGGSLRLSCASSGYISSRSTYCMGWFRQAPGKEREVAAIVTGDS | 210. |
| VHH12 | RTYYGDSVRGRFAISRDNAKNTLYLQMNSLKPEDTAMYYCAAGPPPCITSMPAGGDY | |
| | GYRYWGQGTQVTVSS | |
| hIL28R_ | QVQLQESGGGLVQPGSSLRLSCAASGFTFSNATMSWVRQAPGKEIEWVSAISNSRGT | 211. |
| VHH13 | KYYAAFVKGRFTISRDNAKNTLYLQLNNLKTEDTAMYYCTKDWKTSYSDYDLSDGQG | |
| | TQVTVSS | |
| hIL28R_ | QVQLQESGGGSVQAGGSLRLSCASSGYISRSSYCMGWFRQAPGKEREVAAIVTGDGR | 212. |
| VHH14 | TYYGDSVKGRFAISRDNAKNTLYLQMNSLKPEDTAMYYCVAGPPPCITTMPAGGDYG | |
| | YRYWGRGTQVTVSS | |

TABLE 8
anti-hIL10Rb sdAb VHH DNA SEQUENCE

| | | SEQ ID |
|---|---|---|
| Name | Sequence | NO: |
| hIL10Rb_VHH- | CAGGTGCAGCTTCAGGAATCAGGCGGAGGCAGCGTGCAGGCAGGGGGTAGCCT | 213. |
| 1 | GCGTCTGTCTTGCGCAGCCAGCGGGTACACCTACAGCTCTGGCTGTATGGGCT | |
| | GGTTTCGCCAAGCCCCAGGAAAAGAACGGGAAGCCGTGGCGGCTATCAATAGC | |
| | GACGGCTCCACCTCCTATGCTGACTCCGTCAAAGGACGCTTCACCATTAGTAA | |
| | AGATAACGCCAAGAACACCTTGTACCTTCAGATGAACTCCTTGAAACCGGAGG | |
| | ACACCGCAATGTATTACTGTGCGGCTGAGCCCTACTGCTCAGGAGGCTACCCA | |
| | CGGTGGTCAGTGGCCGAGTTTGGTTATTGGGGCCAGGGCACCCAAGTGACTGT | |
| | GTCCTCC | |
| hIL10Rb_VHH- | CAGGTGCAACTCCAGGAGTCAGGGGGAGGTTCCGTGCAGGCTGGCGGTTCTCT | 214. |
| 2 | CAGGTTGTCTTGCGCGGCCAGCGGCTATACGTACAGTAGCTACTGCATGGGCT | |
| | GGTTCCGGCAAGCCCCCGGCAAGGAGCGCGAAGGCGTGGCTGCCATTGATTCC | |
| | GATGGATCTACTAGGTATGCTGATAGTGTAAAGGGCCGCTTCACAATCTCCAA | |
| | GGACAATGCCAAGAACACACTGTATTTGCAAATGAACTCCCTCAAGCCCGAGG | |
| | ATACCGCTATGTACTATTGCGCTGCCGAACCATACTGTTCCGGTGGCTATAAG | |
| | CGCACTATGGTGGCCGAGTTCGGATACTGGGGTCAAGGCACACAGGTCACAGT | |
| | GTCCTCT | |
| hIL10Rb_VHH- | CAGGTGCAGTTGCAGGAGTCCGGGGGCGGTAGCGTTCAGGCTGGAGGGTCCCT | 215. |
| 3 | GCGTCTGAGTTGTGCGGCATCTCGGTATACTTATAACAGTTACTGTATGGGTT | |
| | GGTTCCGCCAGGCACCTGGAAAGGAGCGGGAGGGGGTGGCGACTATTGATAGC | |
| | GACGGAATGACCAGATATGCCGACTCTGTGAAGGGAAGATTTACTATCTCAAA | |
| | AGATAATGCCAAGAACACACTCTATTTGCAGATGAACAGCCTCAAGCCAGAGG | |
| | ATACCGCTATGTATTACTGTGCTGCCGACGCTGATTGCACCATCGCTGCCATG | |
| | ACGACCAACCCCTTGGGCCAGGGAACCCAAGTAACCGTCTCTAGC | |
| hIL10Rb_VHH- | CAGGTCCAGCTCCAGGAATCTGGTGGCGGGTCTATCCAGGCGGGTGGCAGCCT | 216. |
| 4 | GCGGCTGAGTTGCGCCGCTTCCCGCTACCTGTATAGTATTGATTATATGGCCT | |
| | GGTTCAGGCAGTCACCGGGCAAAGAGCGCGAACCCGTCGCTGTGATTTACACA | |
| | GCCTCTGGTGCCACCTTCTATCCCGATAGTGTGAAGGGCCGGTTCACTATCTC | |
| | TCAAGACAACGCGAAGATGACTGTCTATCTTCAGATGAACTCTCTGAAGTCCG | |
| | AGGACACTGCCATGTATTACTGTGCCGCTGTGCGCAAGACGGACTCTTATCTG | |
| | TTCGATGCCCAGAGTTTCACTTACTGGGGTCAGGGTACTCAGGTGACCGTATC | |
| | CTCC | |
| hIL10Rb_VHH- | CAGGTGCAGCTCCAGGAGTCTGGTGGCGGGCTGGTTCAGCCTGGGGGTTCACT | 217. |
| 5 | CCGGTTGTCCTGCGCTGCGTCTGGTTATACCTACTCCAGCTACTGTATGGGTT | |
| | GGTTCCGCCAGGCACCGGGGAAGGAGAGGGAGGGCGTGGCTCACATTGATTCT | |
| | GATGGCTCTACGACCTACGCTGATAGCGTTAAGGGGCGCTTCACTATCTCCAA | |
| | GGATAACGCCAAGAACACCCTGTATCTGCAAATGAACAGCCTGAAGCCAGAAG | |
| | ACACTGCCATGTACTATTGCGCTGCCGATCCTATTCCCGGTCCTGGCTATTGT | |
| | GACGGCGGTCCTAACAAGTACTGGGGCCAAGGCACACAGGTGACTGTCAGTTC | |
| | C | |
| hIL10Rb_VHH- | CAGGTTCAACTCCAGGAATCCGGCGGTGGAAGCATTCAGGCGGGCGGTTCTTT | 218. |
| 6 | GACTCTGAGCTGTGCGGCATCTCGGGACCTTTACAGCACTAACTATGTTGCCT | |
| | GGTTCCGGCAGTCCCCCGGCAAGGAACGCGAAGCTGTGGCCGTGATTTATACA | |
| | GCCAGCGGCGCAACCCTGTATAGCGATTCAGTCAAAGGCCGGTTCACCATCTC | |
| | CCAGGACAACGCGAAGATGACCGTGTACCTGCAAATGAACAGCCTGAAGTCTG | |
| | AGGACACTGCCATGTATTACTGCGCAGCTGTGAGAAAGACCGGACATTACCTC | |
| | TTCGACGCCCAATCTTTCACCTACTGGGGCCAGGGAACCCAGGTCACCGTCTC | |
| | CTCT | |
| hIL10Rb_VHH- | CAGGTGCAACTCCAGGAGTCAGGCGGTGGGTCCGTCCAGGCCGGTGGCTCCCT | 219. |
| 7 | GAGGCTGAGTTGCGCCGCTTCCGGCTATACTTACTCCAGCGGTTGCATGGGGT | |
| | GGTTCCGCCAAGCCCCCGGTAAAGAACGCGAGGGAGTGGCTACAATTAACTCC | |
| | GATGGAAGCACTAACTACGCCGACTCTGTGAAGGGACGCTTCACCATTAGCAA | |
| | AGACAATGCTAAGAACACCCTTTACCTTCAAATGAACAGCCTGAAGCCTGAGG | |
| | ATACCGCTATGTATTACTGTGCCGCAGAACCGTATTGTAGCGGTGGCTACCCT | |
| | CGCTGGTCCGTCGCCGAGTTCGGTTATTGGGGCCAGGGGACCCAAGTGACTGT | |
| | TTCTAGC | |
| hIL10Rb_VHH- | CAGGTGCAACTTCAGGAGAGCGGCGGGGGCTCTGTGCAAGCTGGTGGCTCCCT | 220. |
| 8 | GCGGCTCAGCTGTGCTGCCTCTGGGTATTCTTACAGTAGCTACTGTATGGGCT | |
| | GGTTCAGACAGGCACCAGGCAAGGAGCGCGAGGGTGTGGCGGCCATCGCTTCC | |
| | GACGGGAGTACCAGCTACGCCGACAGCGTTAAAGGTAGGTTTGCCATCTCTAA | |
| | GGATAATGCGAAGAATACACTCTACCTTCAGATGGCTAGTCTGAAGCCAGAGG | |
| | ATACCGCCATGTATTACTGCGCGGCAGAGCCCTGGTGCACGGGAGGGTATTCA | |
| | CGCCTGACCCCGGCTGAGTATGGATACTGGGGGCAGGGCACCCAGGTGACCGT | |
| | TAGCTCC | |
| hIL10Rb_VHH- | CAGGTCCAGTTGCAGGAAAGCGGAGGGGGCCTGGTGCAGCCAGGAGGTTCTCT | 221. |
| 9 | GAGACTGAGCTGTGCCGCTTCTGGTTACACATATTCTAGCGGGTGCATGGGCT | |
| | GGTTCCGCCAGGCTCCCGGCAAGGAACGTGAGGGTGTGGCAACTATCAATTCC | |
| | GACGGCTCTACAAACTACGCAGATTCTGTTAAAGGCCGCTTCACAATCTCTAA | |
| | GGACAACGCCAAAAACACTCTGTACTTGCAGATGAATAGCCTGAAGCCTGAAG | |
| | ACACTGCCATGTACTATTGCGCAGCTGAGCCCTACTGTTCTGGAGGCTACCCC | |
| | CGCTGGTCTGTGGCCGAGTTCGGTTACTGGGGACAAGGAACCCAGGTCACAGT | |
| | GTCCAGT | |
| hIL10Rb_VHH- | CAGGTTCAGCTCCAGGAGTCAGGCGGGGGTCTTGTCCAGCCTGGTGGCTCCCT | 222. |
| 10 | GCGCCTGTCCTGTGCTGCCTCCGGTTACACCTACTCCAGCTATTGCATGGGAT | |
| | GGTTCAGACAAGCGCCAGGCAAGGAACGTGAGGGGGTCGCCCACATTGACTCC | |
| | GACGGTTCCACTACCTACGCCGACAGCGTCAAAGGCCGCTTCGCGATTTCTAA | |
| | GGATAACGCTAAGAATACTCTGTACTTGCAGATGAACTCACTGAAGCCAGAGG | |
| | ACACGGCCATGTATTACTGCGCAGCCGATCCGATCCCCGGCCCCGGCTATTGT | |
| | GACGGTGGCCCGAACAAGTACTGGGGACAGGGCACCCAAGTGACGGTGTCCTC | |
| | T | |
| hIL10Rb_VHH- | CAGGTACAGTTGCAGGAGAGCGGAGGCGGTTCCGTGCAGGCAGGTGGCTCTCT | 223. |
| 11 | TAGACTGTCCTGCGCCGCGAGCGGGTACACCTACAGTAGCTATTGTATGGGCT | |
| | GGTTCCGCCAGGCTCCTGGTAAGGGTCGCGAGGGCGTCGCTGCCATCGACTCC | |
| | GATGGCTCTACTCGCTACGCAGATTCTGTCAAGGGGCGCTTCACAATTTCCAA | |
| | GGACAACGCCAAGAACACGCTTTACTTGCAGATGAACTCACTGAAGCCGGAGG | |
| | ACACCGCTATGTATTACTGCGCTGCCGAGCCCTACTGTTCTGGGGGCTACAAG | |
| | CGCACTATGGTGGCCGAGTTCGGATATTGGGGCCAGGGTACACAGGTGACCGT | |
| | CAGTTCT | |
| hIL10Rb_VHH- | CAGGTGCAGTTGCAGGAGTCTGGCGGTGGCTCTGTGCAGGCTGGGGGCTCTCT | 224. |
| 12 | GCGCCTGAGTTGCGCTGCCAGCGGTTACACCTACTCCAGCTATTGTATGGGAT | |
| | GGTTCCGCCAGGCTCCGGGGAAGGAGAGGGAGGGCGTGGCCCATATCGACTCT | |
| | GATGGCTCCACATCCTACGCCGACAGCGTGAAGGGACGTTTCACCATTAGCAA | |
| | GGACAATGCGAAGAATACCCTCTACTTGCAGATGAACTCCCTGAAGCCGGAGG | |
| | ATACTGCCATGTATTACTGCGCCGCTGATCCCATCCCAGGGCCTGGGTACTGT | |
| | GACGGAGGCCCGAACAAGTATTGGGGACAAGGAACGCAGGTCACAGTGTCATC | |
| | T | |
| hIL10Rb_VHH- | CAGGTACAACTCCAGGAGAGTGGTGGAGGCTCCGTTCAAGCCGGGGGCTCCCT | 225. |
| 13 | GCGGCTGTCCTGTGCGGCCAGCGGTTACACCTATTCATCTTACTGTATGGGCT | |
| | GGTTCCGGCAGGCCCCTGGTAAGGAAAGAGAGGGTGTCGCTCACATTGATTCC | |
| | GACGGTAGTACCTCTTACGCAGACTCTGTCAAGGGCAGGTTCACCATCTCTAA | |
| | GGACAATGCCAAGAACACCTTGTACCTCCAGATGAACTCTCTGAAGCCCGAGG | |
| | ACACTGCAATGTACTATTGTGCGGCTGACCCTATTCCCGGCCCTGGATATTGC | |
| | GACGGCGGACCTAACAATTACTGGGGACAGGGCACCCAGGTCACCGTCAGCTC | |
| | C | |
| hIL10Rb_VHH- | CAGGTTCAGCTCCAAGAATCCGGCGGGGGCTCTGTGCAGGCGGGCGGAAGTCT | 226. |
| 14 | GCGTCTGTCATGCGCTGCCAGCGGGTACACTTACTCTTCCGGTTGTATGGGCT | |
| | GGTTTAGGCAGGCTCCGGGAAAGGAAAGGGAGGGCGTCGCAACTATCAACAGC | |
| | GACGGCTCTACGAACTACGCTGACTCTGTGAAAGGCCGCTTCACCATCAGCAA | |
| | AGACAACGCCAAAAATACACTGTATCTCCAGATGAATAGCTTGAAACCCGAGG | |
| | ACACCGGAATGTATTACTGCGCGGCAGAGCCATACTGTTCAGGCGGTTACCCA | |
| | AGATGGTCCGTGGCTGAGTTCGGTTATTGGGGGCAGGGCACTCAGGTTACTGT | |
| | GTCTTCC | |
| hIL10Rb_VHH- | CAGGTGCAGCTCCAGGAATCCGGGGGCGGTTCTGTGCAGGCTGGTGGCTCTCT | 227. |
| 15 | GCGCCTGTCTTGCACTGTTTCCAGGTACACTGCCTCTGTAAACTATATGGGCT | |
| | GGTTTAGACAAGCTCCGGGCAAGGAACGCGAAGGCGTCGCTACCATCTTTACA | |
| | GGTGCAGGTACGACCTATTACGCCAATAGCGTTAAAGGGAGGTTCACCATCTC | |
| | CAGGGACAATGCCAAAAACACAGCCTATCTCCAGATGAACTCCCTCAAACCTG | |
| | AAGACACAGCCATCTACTATTGCGCGGTTGACTTCCGTGGTGGCCTGCTCTAT | |
| | AGACCGGCGTATGAGTACACCTACCGTGGACAAGGCACCCAAGTCACAGTGAG | |
| | CAGC | |
| hIL10Rb_VHH- | CAGGTGCAGCTCCAAGAGTCCGGCGGAGGGAGTGTAGAGGCTGGCGGGTCCCT | 228. |
| 16 | GCGCCTTAGCTGCGCGGCCAGCGGCTATACACACAGTTCTTATTGTATGGGTT | |
| | GGTTCCGCCAAGCTCCGGGAAAGGAGCGTGAGGGCGTGGCTGCCATCGACGTG | |
| | GATGGCTCCACAACCTACGCCGACAGCGTGAAGGGCAGGTTTACGATCTCTAA | |
| | GGATAACGCTAAGAATACTCTCTATTTGCAGATGAACTCCCTCAAACCCGAGG | |
| | ATACAGGAATGTACTATTGCGCTGCCGAGTTCGCCGACTGCTCAAGCAATTAT | |
| | TTCCTGCCTCCAGGAGCCGTTAGGTACTGGGGCCAGGGGACTCAGGTCACAGT | |
| | AAGCAGC | |
| hIL10Rb_VHH- | CAGGTGCAGCTCCAGGAGAGCGGTGGCGGATCAGTGCAGGCTGGAGGCTCCCT | 229. |
| 17 | CAGACTGTCCTGCACCGTGAGCCGCTATACCGCCTCCGTCAACTATATGGGAT | |
| | GGTTTAGGCAGGCTCCGGGCAAGGAGCGCGAGGGGGTCGCGACTATCTTCACC | |
| | GGAGCCGGTACTACCTATTACGCTAATTCTGTTAAAGGCCGCTTTACCATTAG | |
| | TCGCGACAACGCTAAGAACACAGCTTACCTCCAGATGAACTCTCTGAAGCCAG | |
| | AGGATACCGCCATGTATTACTGCGCCGTGGACTTCCGGGGCGGTTTGCTCTAC | |
| | CGCCCGGCCTACGAATACACCTATCGCGGCCAGGGCACGCAGGTCACGGTGTC | |
| | CTCA | |
| hIL10Rb_VHH- | CAGGTGCAGCTCCAAGAGTCCGGTGGAGGCAGCGTCCAGGCCGGGGGTAGTCT | 230. |
| 18 | TAGGCTCAGCTGTGCTGCCAGTGGAGACACCTACTCTTCCTATTGCATGGGAT | |
| | GGTTCAGACAGGCCCCCGGCAAAGAGCGCGAGGGCGTTGCATTCATCGACTCC | |
| | GACGGCTCCACTCGCTACGCCGATAGCGTGGAGGGCCGTTTTACCATCTCCAA | |
| | GGACAACGCGAAGAACACTCTGTATCTGCAAATGAACTCCCTGAAGCCCGAAG | |
| | ACACCGCCATGTACTATTGCGCGGCTGAGCCATACTGTAGTGGCGGATATCAT | |
| | CGTAAGGAAATGGCAGAGTTCGGCTATTGGGGCCAGGGCACCCAGGTCACTGT | |
| | GAGTTCC | |
| hIL10Rb_VHH- | CAGGTGCAGTTGCAGGAATCCGGCGGAGGCTCTGTGCAGGCGGGCGGTTCCCT | 231. |
| 19 | CCGCCTGAGTTGTGCCGCGTCTGGCTATACTTACTCTTCCTATTGTATGGGAT | |
| | GGTTCCGGCAAGCGCCCGGCAAAGAGCGGGAGGGCGTTGCGCATATCGACAGT | |
| | GATGGTAGCACCAGTTACGCTGATAGCGTGAAAGGCAGATTCACTATCTCAAA | |
| | GGATAACGCGAAGAACACTCTTTACCTCCAGATGAACTCCCTTAAACCTGAGG | |
| | ATACCGCGATGTATTACTGTGCTGCCGACCCCATTCCCGGCCCTGGATACTGT | |
| | GACGGAGGCCCTAACAAGTACCGTGGGCAAGGAACACAGGTCACAGTGTCCAG | |
| | C | |
| hIL10Rb_VHH- | CAGGTGCAACTCCAGGAGTCTGGCGGGGGCAGCGTCCAGGCAGGTGGAAGTCT | 232. |
| 20 | CCGTCTCTCATGTGCTGCCAGCGGCTATACATACTCCAGCTACTGTATGGGAT | |
| | GGTTTAGACAGGCACCCGGCAAGGAGCGCGAAGGGGTGGCCCATATCGACTCC | |
| | GATGGCAGCACAACCTATGCCGACTCTGTGAAAGGGCGGTTCGCCATCTCCAA | |
| | GGACAACGCTAAGAATACCCTGTACCTCCAGATGAACTCTCTGAAGCCTGAGG | |
| | ACACCGCCATGTATTACTGCGCTGCCGACCCAATCCCTGGCCCAGGTTACTGC | |
| | GATGGGGGACCAAACAAATATTGGGGACAGGGCACGCAGGTTACAGTCTCCAG | |
| | C | |
| hIL10Rb_VHH- | CAGGTCCAACTCCAGGAAAGTGGAGGTGGCTCTGTTCAGGCCGGGGGCAGCCT | 233. |
| 21 | GAGGCTGAGCTGCACCGGCTCAGGCTATACAGCCAGTAATAACTGCATGGGCT | |
| | GGTTCCGTCAAGCGCCCGGCAAAGAGCGTGAAGGTGTGGCCGTAATTTTTACC | |
| | GGCGCTGGCACCAGCTATTACGACAGTTCCGTGGGCCGTCTGTTCATCAGCTC | |
| | ACAGGACGCCGCTTCCACCCTCGATCAGTTGCTGATGAGCCTTCTGCCCGATG | |
| | ACACCGCAGTAATGTACTGTGGAGCCGAAGATGACTGCACACTGCTCCTGATG | |
| | ACGCCAAACCCCGATGACCAATGGTCCCGCCTGAGTGTGTCCTCC | |
| hIL10Rb_VHH- | CAGGTGCAGCTCCAGGAGAGCGGGGGCGGTTCTGTTCAGGCGGGAGGCAGCCT | 234. |
| 22 | GCGTCTGTCCTGTGCAGCCTCTGGTTACACAGACAGTCGTTACTGCATGGGCT | |
| | GGTTCCGCAAGGCACCTGGAAAGGAGCGCGAGGGTGTTGCGCACATCGACTCC | |
| | GACGGGAGCACTAGCTATGCTGACAGCGTGAAGGGGCGCTTCACTATCAGCAA | |
| | GGATAACGCGAAAAACACCTTGTACCTTCAGATGAACTCCCTCAAACCCGAAG | |
| | ACACAGCGATGTACTATTGTGCCGCTGATCCGATCCCAGGGCCTGGCTACTGT | |
| | GATGGTGGACCTAATAAGTACTGGGGGCAGGGAACTCAGGTGACCGTGTCATC | |
| | A | |
| hIL10Rb_VHH- | CAGGTCCAGTTGCAGGAATCTGGAGGCGGTTCCGTGCAAGCAGGGGGCTCACT | 235. |
| 23 | CAGACTGTCCTGCGCTGCCAGCGGCTACACTTACTCTTCATATTGCATGGGCT | |
| | GGTTCCGCCAGGCACCGGGCAAGGAGCGGGAAGGCGTGGCCGCTATTGATAGC | |
| | GATGGCTCTACGCGCTACGCAGATAGCGTGAAAGGGAGGTTCACGATCTCCAA | |
| | AGATAATGCCAAGAAAATTCTGTATCTCCAGATGAACTCTCTGAAGGTCGAGG | |
| | ACACCGCCATGTACTATTGTGCAGCCGAACCCTACTGTTCTGGTGGCTACAAG | |
| | AGGACTATGGTGGCCGAGTTCGGCTTCTGGGGCCAGGGGACCCAAGTGACTGT | |
| | CAGTAGC | |
| hIL10Rb_VHH- | CAGGTGCAACTTCAGGAGAGCGGTGGCGGATCTGTGCAGGCTGGAGGGTCTCT | 236. |
| 24 | GAAGCTGTCCTGCGCGGCCAGCGGTTACACATACAGTAGCTACTGCATGGGAT | |
| | GGTTTCGTCAGGCCCCAGGCAAGGAGCGCGAAGGAGTGGCGCACATCGACTCC | |
| | GATGGGTCCACCACATACGCCGACTCCGTGAAGGGCCGTTTCACAATCAGCAA | |
| | GGATAACGCGAAGAACACGCTGTACTTGCAGATGAACTCTCTCAAACCAGAGG | |
| | ACACTGCAATGTACTATTGCGCGGCTGACCCCATCCCTGGCCCTGGTTACTGT | |
| | GACGGTGGCCCCAACAATTACTGGGGGCAAGGGACCCAAGTCACCGTGTCCTC | |
| | C | |
| hIL10Rb_VHH- | CAGGTCCAGCTCCAGGAGTCCGGCGGGGGCTCCGTCCAGGCAGGGGGCTCCCT | 237. |
| 25 | GCGTCTGTCATGCGCCGCTTCTGGGTATACCTACAGTTCCTATTGTATGGGTT | |
| | GGTTTCGCCAAGCACCCGGTAAGGAGCGCGAAGGTATTGCGCACATTGATAGC | |
| | GATGGCTCCACAACCTATGCTGACAGTGTGAAAGGACGCTTCACTATTTCCAA | |
| | GGATAACGCTAAGAACACACTCTACCTTCAGATGAACAGCCTGAAGCCGGAAG | |
| | ACACCGCAATGTACTATTGTGCAGCTGACCCCATTCCTGGACCCGGTTACTGT | |
| | GATGGAGGTCCTAATAACTATTGGGGACAGGGCACTCAAGTGACCGTCTCAAG | |
| | C | |
| hIL10Rb_VHH- | CAGGTGCAGTTGCAGGAGAGCGGGGGTGGCTCTGTGCAGGCCGGGGGCTCCCT | 238. |
| 26 | GAGGCTGAGCTGCGCGGCCAGCGGGTACAGCTACTCTAGCTATTGCATGGGTT | |
| | GGTTCCGCCAGGCCCCTGGCAAGGAGCGCGAGGGAGTGGCCACGATTGACTCA | |
| | GATGGCATGACCCGTTATGCGGATTCCGTCAAGGGGCGCTTCACCATCAGCAA | |
| | AGATAACGCCAAAAATACCCTGTACTTGCAGATGAACTCACTGAAACCTGAGG | |
| | ATACAGCCATGTATTACTGCGCAGCTCCGCTCTATGACTGTGACTCTGGTGCC | |
| | GTGGGTAGAAACCCACCTTACTGGGGGCAGGGAACCCAGGTGACCGTGTCCTC | |
| | A | |
| hIL10Rb_VHH- | CAGGTCCAGCTCCAGGAAAGCGGTGGGGGCAGCGTCCAAACAGGGGGTAGCCT | 239. |
| 27 | GCGCCTCTCTTGCGCAGCCAGCGGCTACACATATCTGCGCGGATGTATGGGCT | |
| | GGTTCCGCCAGGCCCCTGGTAAGGAAAGAGAGGGGGTGGCCGTGATGGACGTG | |
| | GTTGGAGACAGACGTTCCTACATTGATTCCGTGAAGGGCCGCTTTACTATCTC | |
| | ACGCGATAACGCGGCTAACTCTGTGTATTTGCAGATGGATAACCTGAAGCCCG | |
| | AGGACACCGCTATGTACTATTGCACAGCTGGTCCCAACTGTGTCGGTTGGCGC | |
| | TCCGGCCTGGACTATTGGGGTCAGGGAACCCAGGTTACAGTTAGCAGT | |

TABLE 9
anti-mIL 10Rb sdAb VHH DNA SEQUENCE

| | | SEQ ID |
|---|---|---|
| Name | Sequence | NO: |
| DR132 | CAGGTGCAGCTCCAGGAGAGTGGTGGCGGTTCTGTCCAAGCTGGCGGAGCCCTGCGCC | 240. |
| 2 aa | TGTCCTGCACAGCAAGCGGCTACACCGCCTCTAGCATTTGCATGGGATGGTTCCGTCA | |
| | GGCCCCAGGCAAGGAGAGGGAGAGAGTGGCTGTGATTACCACGGCAGCCTCCGGTACT | |
| | TACTATGCCGACTCTGTGAATGGCCGCTTCTCAATCTCTCAGAATAACGCCAAAAATA | |
| | CTGTGTACCTCCAGATGAACTCCCTGAAACCTGACGATACCGCGATGTATTACTGCGC | |
| | AGCCACCCGGCGCGGCGGTGACTGCCTGGACCCATTGCAGACCCCAGCCTATAATACC | |
| | TGGGGCCAGGGAACCCAGGTCACCGTCTCTTCT | |
| DR132 | CAGGTGCAGCTCCAGGAAAGCGGCGGTGGCTCCGTCCAGGCCGGTGGCTCCCTGAGGC | 241. |
| aa | TGAGCTGTGTGGCTTCCGGCGATACTTATTCTCGCAAGTACATCGCATGGGTGCGTCA | |
| | GGTGCCCGGTAAAGAACGTGAGGGAGTGGCAGTGATGTATACCCCAGGCTCCGCTACT | |
| | TACTATACAGACACAGTGATGGGTCGTTTCACCATCTCCCAGGACAACGCCAAGAACA | |
| | CTGTGTACCTTCAAATGAACAGCCTCAAACCTGAAGACACCGCCATGTACTTTTGCGC | |
| | GGCCAAGGCCAGCGGCTCCATGTTTAACTTCCGCGATTACACTTATTGGGGACAGGGC | |
| | ACTCAGGTGACCGTAAGCTCT | |
| DR132 | CAGGTGCAGCTGCAAGAAAGCGGAGGTGGCTCTGTCCAGGCAGGAGGCTCCCTCCGGC | 242. |
| 4 aa | TTAGCTGCGCTACCAGCGGGTATGCTTCCTGTTCCCGCGCCATGAGGTGGTACAGGCA | |
| | GGCACCGGGCAAGGAGCGCGAATTTGTGGCGTACATCGACGGGGTGGGCAGTACTGGT | |
| | TATGCGGACAGCGTTAAAGGCCGGTTTACCATCTCCCAAGATAATGCAAAGTACACGG | |
| | CTTACTTGCAGATGAACTCCCTCAAGCCTGAGGATACCGCGATGTATTACTGTAATCG | |
| | GGGCTGTAGAGCCGATGGTAGCAATAGTCTGGACAACTACTGGGGCCAGGGCACACAG | |
| | GTGACTGTCTCTTCA | |
| DR132 | CAGGTGCAGTTGCAGGAGTCCGGCGGTGGCAGCGTTCAGGCGGGCGGTAGCCTGCGTC | 243. |
| 5 aa | TGAGCTGCGCCGCGTCCGGCTACACCTATAACCGTCGCTTCATGGGTTGGTTCCGTCA | |
| | AGCGCCCGGCAAGGAGAGAGAGGGCCTCGCCATTATCTACACCCCCAACAGCTCCACC | |
| | TTCTACGCCGACTCTGTGACGGGCCGCTTTACAATCTCACAGGATTCTGCCCGCAACA | |
| | CCGTCTATTTGCAGATGAACTCCCTGAAACCTGAGGACACCGCTATGTACTATTGTGC | |
| | AGCCGCTCGCATCGCTTCTATGACTGAGCTTTCAGTGAGAGATATGGACTATTGGGGC | |
| | AAGGGCACCCAGGTGACCGTTTCCTCC | |
| DR132 | CAGGTACAACTCCAGGAGAGCGGGGGAGGTAGCGTACAGGCTGGCGGGTCCTTGCGTC | 244. |
| 6 aa | TGAGCTGCACTGCATCTCGTTACATCGCTCTTAATGCGTGTATGGCTTGGATTCGGCA | |
| | GGCCCCCGGCTCCGAAAGGGAGGTCGTGGCCACAATCGTGACTGATGGCTCCAGAACC | |
| | TATTACGCAGACTCTGTCAAGGGCCGGTTTACTATCTCTCAAGACAACGCCAAGAACA | |
| | CCATGTACCTCCAGATGAACGGTTTGAAACCCGAAGACACCGCCATGTATTACTGTGC | |
| | AGCCGACAGGCGCTGCCCCGTGTCCAGAGCCCCATACGAATACGAACTGCGCTACTGG | |
| | GGTCAGGGCACCCAGGTGACTGTCAGCAGC | |
| DR132 | CAAGTCCAGCTTCAAGAAAGCGGAGGGGGCTCTGTTCAGGCAGGCGGGTCCCTCCGGC | 245. |
| 7 aa | TGTCCTGCGCTGCCTCCGGCTACACATACAACGGAAAGTGCATGGCTTGGTTCCGCCA | |
| | GGCTCCCGGCAAGGAGCGCGAAGTCGTGGCTGGCATTTACACCGGGGGCTCCAGCACA | |
| | TATTACGCCGATAGTGTGAAGGGACGCTTTACGATTTCCCAAGACAATGCTAAAAATA | |
| | CAGTCTATCTCCAGATGGACAGCCTGAAGCCCGAAGACACTGCCATGTATTACTGCGC | |
| | CACCAGCAGAAGCTGTAGCGACCTGCGCAGACGCTCCATCGCCTACTGGGGACAGGGG | |
| | ACTCAGGTCACCGTCAGCTCT | |

TABLE 10
anti-hIL28RA sdAb is a VHH DNA SEQUENCE

| | | SEQ ID |
|---|---|---|
| Name | Sequence | NO: |
| hIL28R_ | CAGGTCCAGTTGCAAGAGAGTGGTGGCGGATCAGTACAGGCTGGGGGCAGTCTGCGCC | 246. |
| VHH | TCTCTTGTGCTTCCTCTGGCTATATCTCCTCTAGTTACTGTATGGCCTGGTTCCGTCA | |
| 1 | GGCTCCCGGTAAAGAGCGTGAGGGTGCTGCGGGCGTGACCAGAGACGGCAAGACCTAT | |
| | TACGGCGACTCTGTAAAGGGCCGGTTCGCGATCTCTCGCGACAACGCTAAGAACACTT | |
| | TGTATCTCCAGATGAACAGCCTGAAACCCGAGGACACCGCTATGTATTACTGTGCCGC | |
| | AGGCCCTCCGCCTTGCATCACCTCCATGCCTGCGGGCGGAGACTATGGTTACCGCTAC | |
| | TGGGGCCAGGGAACACAGGTGACTGTGTCCTCC | |
| hIL28R_ | CAGGTGCAACTTCAGGAGAGCGGTGGAGGTCTGGTCCAACCAGGAGGCTCACTCCGCC | 247. |
| VHH | TGTCCTGCGCGGCCAGTGGTTTTACTTTTTCTAACTACGGCATGTCTTGGGTGCGCCA | |
| 2 | GGCACCAGGCAAGGGCCTGGAGTGGGTAAGCGGGATCAATAGTGGCGGAGATGACACC | |
| | TTCTACACGGACAGCGTGAAGGGCCGCTTCACTATCAGTAGGGATAACGCTAAGAACA | |
| | CTCTCTACTTGCAGATGAACTCCCTGAAGACCGAAGACACCGCCATGTATTACTGTGC | |
| | TATGGGAGCCAGTGGGATGATCCCTCGCGGTCAGGGCACCCAAATCACTGTCAGTTCT | |
| hIL28R_ | CAAGTACAGCTCCAGGAGAGTGGCGGTGGGCTCGTGCAACCCGGAGGTTCCCTGAGAT | 248. |
| VHH | TGTCCTGTGTGGCCTCTGGTTTTACCTTCTCTGATTACGCCATGAGCTGGGTGCGCCA | |
| 3 | AGCTCCAGGAATGGGATTGGAACGGGTCTCTGCAATCGGTCGCGACGGCTCCACTTTC | |
| | TACCCTGACTCTGTGAAAGGCCGCTTCACAATCTCTCGCGATAACGCCAAGAATACCC | |
| | TGTACCTCCAGCTGAACAGTTTGAAGACCGAAGATACTGCTATGTATTACTGTGCCAA | |
| | AGAAGAGCCAGGTTCCTCTTCACGCGGACAGGGAACCCAGGTGACAGTCTCTTCC | |
| hIL28R_ | CAGGTGCAGCTTCAGGAGTCTGGTGGGGGCAGCGTGCAGCTGGGTGGCAGTCTGCGTC | 249. |
| VHH | TGAGTTGTCTGGTGAGTGGCAGTACTGACAACATCAAGTACATGGGCTGGTTTCGCCA | |
| 4 | GGCCCCTGGCAAAGAGCGCGAAGGAGTGGCCGCTGTGTATACGTCCGGCGGTGCGGTT | |
| | GTGTACGCCGATAGTGTGAAGGGCAGGTTCACCATTAGTCAGGATGACGCTAAGAACA | |
| | CCATGTACCTCCAGATGAACTCCCTGAAGCCAGAAGATACCGCTATGTACTATTGCGC | |
| | TGCGTCCCGTGCTCCCGCACCCCCTCGCCTTCTGTTGCAGCGGGCGCTGGTGGAATAT | |
| | TGGGGCCAGGGGACCCAGGTGACCGTCAGTAGC | |
| hIL28R_ | CAGGTCCAGTTGCAAGAGAGCGGTGGGGGCCTGGTTCAGCCTGGTGGGAGCCTGCGTC | 250. |
| VHH | TCTCCTGTGCCGCTTCTGGCTTCACCTTTTCTAACGCCACAATGAGCTGGGTCCGCCA | |
| 5 | AGCGCCGGGTAAAGAAATCGAATGGGTCAGTGCAATCTCAAACAGCAGAGGCACGAAA | |
| | TATTACGCAGCCTTCGTCAAGGGGCGCTTCACGATTTCCCGCGATAATGCTAAAAATA | |
| | CACTGTACCTCCAGCTTAATAACCTGAAGACCGAGGACACCGCAATGTACTATTGTAC | |
| | TAAGGACTGGAAAACAAGCTATTCTGACTATGACCTGTCTGATGGCCAAGGCACTCAG | |
| | GTGACCGTCAGTAGC | |
| hIL28R_ | CAGGTCCAGCTCCAGGAAAGCGGGGGCGGTTTGGTGCAGCCCGGTGGGAGCCTGAGAC | 251. |
| VHH | TGAGCTGTGCTGCCTCTGGCTTTACCTTTTCCGACTACGCCATGTCCTGGGTCCGTCA | |
| 6 | GGCCCCCGGAATGGGTCTGGAGAGAGTGTCTGCCATCGGCAGGGACGGCTCTACCTTC | |
| | TACCCGGATTCCGTAAAAGGCCGCTTCACCATCTCCCGTGACAACGCGAAGAACACAC | |
| | TGTACTTGCAGCTCAACTCCCTGAAGACTGAAGACACCGCGATGTATTACTGCGCTAA | |
| | GGAAGAGCCAGGCTCTTCAAGTAGAGGCCAGGGTACTCAGGTGACAGTGTCTAGC | |
| hIL28R_ | CAGGTGCAGCTTCAGGAGTCCGGGGGAGGCCTGGTCCAGCCCGGTGGCTCACTGCGCC | 252. |
| VHH | TGTCTTGTGCCGCTTCCGGCTTTACCTTCTCTAACTACGGCATGAGCTGGGTTAGGCA | |
| 7 | GGCACCCGGCAAAGGTCTGGAGTGGGTGTCTGGTATCAATTCTGGAGGCGATGACACA | |
| | TTTTACACAGATTCAGTCAAGGGCCGCTTCACTATCTCCAGGGATAACGCTAAGAACA | |
| | CTCTGTACCTCCAAATGAACTCCCTGAAGACGGAAGACACCGCTATGTACTATTGCGC | |
| | GATGGGCGCTTCCGGTATGATCCCGCGCGGACAAGGCACCCAGGTGACTGTAAGTTCT | |
| hIL28R_ | CAGGTACAGCTCCAGGAAAGTGGCGGTGGCTCCGTCCAGGCGGGCGGAAGCCTGCGGC | 253. |
| VHH | TGTCCTGCGCCGTGTCCCGCTATACAATTAGCCGGTCAGATTGTATGGGCTGGTTCCG | |
| 8 | TCAAGCCCCAGGGAAGGAGAGGGAGGGTGTCGCCCGTATCGGCAGCGACGGCACGACA | |
| | AGCTATGCGGACTCCGTCAAGGAGCGTTTTACCATCTCTAAGGACAACGCAAAGAACA | |
| | TCCTGTACCTCCAGATGAACAGTCTCAAGCCCGAGGACACTGCCCGTTATTACTGTGC | |
| | TGCCACCGCCCTGCTTCTGGGAAGAGGCTCAGCGTGTCACAAAGAGGTGTCAGTGTTC | |
| | TCTTGGTGGGGCCAGGGCACCCAGGTGACTGTGTCTTCC | |
| hIL28R_ | CAGGTCCAACTCCAGGAGTCTGGCGGGGGCCTGGTCCAGCCAGGAGGCTCCCTCCGTC | 254. |
| VHH | TCTCCTGCGCCGCTTCCGGCTTCACCTTCAGTAATTACGGCATGAGTTGGGTTCGCCA | |
| 9 | GGCTCCCGGCAAGGGCCTGGAGTGGGTCTCAGGTATCAATTCTGGGGGCGACGATACA | |
| | TTCTATACAGACTCCGTTAAGGGCCGCTTTACGATTAGCCGCGATAACGTGAAGAATA | |
| | CCCTTTATCTCCAGATGAACTCCCTGAAGACCGAAGATACTGCGATGTATTACTGCGC | |
| | AATGGGGGCCTCCGGTATGATTCCTAGAGGCCAGGGCACCCAAGTGACCGTCAGCAGT | |
| hIL28R_ | CAGGTGCAGCTCCAGGAAAGTGGAGGGGGCTCTGTCCAGGCAGGCGGTAGTCTGCGCC | 255. |
| VHH | TCTCCTGCGCCTCTAGCGGTTACATTTCCTCTCGCTCTACCTACTGTATGGGATGGTT | |
| 10 | CCGCCAGGCTCCGGGCAAGGAACGCGAGGTGGCGGCAGTTGTGACCGGGGACTCTCGT | |
| | ACCTACTATGGTGACTCAGTGAAGGGCCGCTTTGCGATTAGTCGCGACAATGCGAAGA | |
| | ACACCCTGTACCTCCAGATGAACTCTCTGAAGCCTGAGGACACTGCCATGTACTATTG | |
| | CGCGGCTGGACCCCCTCCCTGCATTACCACTATGCCCGCTGGGGGTGACTACGGGTAT | |
| | CGGTATTGGGGTCAAGGCACCCAGGTGACAGTTAGCAGC | |
| hIL28R_ | CAGGTGCAGTTGCAGGAGTCTGGCGGAGGCTCCGTGCAGTCCGGCGGGAGCTTGCGCC | 256. |
| VHH | TCTCTTGCGCTGCCTCTGGATTCACGTACTCAAGCTACTGTATGGGCTGGTTTCGCCA | |
| 11 | AGCGCCAGGTAAGGAACGCGAAGGCGTGGCAGCCATTGATTCCGACGGTTCCACTTCT | |
| | TATGCTGACAGCGTCAAGGGTAGATTCACTATCTCTAAGGACAACGCTAAGAACACCC | |
| | TGTACCTCCAGATGAACTCCCTGAGACCTGAAGATACCGCTATGTATTACTGTGCGGC | |
| | AGACGGCGAGTATAACGATTACGTTTGCTGGAGCACTGGTCTTCGGTATCGGGGACAG | |
| | GGTACACAGGTGACCGTGAGCAGT | |
| hIL28R_ | CAGGTCCAGTTGCAGGAGTCAGGAGGTGGCTCCGTGCAAGCCGGGGGCTCCCTTCGCC | 257. |
| VHH | TGTCTTGCGCTAGTAGCGGATACATCAGTTCCCGCTCCACATATTGTATGGGCTGGTT | |
| 12 | CCGTCAAGCGCCCGGCAAAGAGCGCGAGGTGGCGGCAATCGTGACGGGTGATTCCAGG | |
| | ACCTACTATGGTGACAGCGTGCGCGGTCGTTTTGCCATCAGCCGGGATAACGCGAAGA | |
| | ATACACTTTACCTCCAGATGAATAGCCTGAAGCCAGAGGATACCGCCATGTATTACTG | |
| | TGCCGCTGGGCCTCCCCCTTGTATCACATCAATGCCTGCTGGGGGCGATTACGGCTAC | |
| | AGATACTGGGGTCAGGGGACCCAGGTGACCGTGTCTTCA | |
| hIL28R_ | CAAGTGCAGCTCCAGGAGTCCGGTGGCGGGCTGGTGCAGCCTGGCAGTTCCCTGCGCC | 258. |
| VHH | TGTCCTGCGCGGCCAGTGGATTCACCTTCTCCAACGCTACTATGTCTTGGGTCCGCCA | |
| 13 | AGCTCCTGGGAAGGAGATCGAATGGGTGTCTGCAATCTCTAATAGCAGGGGAACCAAG | |
| | TACTATGCGGCTTTCGTGAAGGGGCGTTTCACCATCTCTCGTGACAACGCCAAAAACA | |
| | CCTTGTACCTGCAACTGAACAATCTGAAAACCGAAGATACCGCCATGTATTACTGCAC | |
| | TAAAGATTGGAAAACGTCCTACTCCGATTACGATCTGAGTGATGGCCAGGGAACTCAA | |
| | GTGACCGTCTCTAGC | |
| hIL28R_ | CAGGTGCAGCTCCAGGAAAGTGGAGGCGGGAGCGTGCAGGCAGGCGGGTCACTCAGAC | 259. |
| VHH | TGTCTTGTGCGTCTAGCGGGTATATCTCTCGTAGCTCCTATTGCATGGGATGGTTTCG | |
| 14 | CCAGGCTCCAGGAAAGGAACGTGAAGTTGCCGCTATCGTGACAGGTGACGGACGTACC | |
| | TATTACGGCGACTCTGTCAAGGGCCGCTTCGCGATCAGCCGTGATAATGCCAAGAACA | |
| | CCCTTTATTTGCAGATGAACAGTCTGAAGCCCGAGGATACTGCTATGTACTATTGTGT | |
| | GGCTGGACCCCCACCTTGCATCACCACTATGCCAGCCGGTGGCGACTATGGATACAGG | |
| | TACTGGGGACGCGGCACCCAGGTCACAGTCTCTAGC | |

1. An IFNlambda receptor 1 (IFNlambdaR1) binding molecule that specifically binds to IL10Rb and IL28RA,
wherein the binding molecule causes the multimerization of IL10Rb and IL28RA when bound to IL10Rb and IL28RA, and
wherein the binding molecule comprises a single-domain antibody (sdAb) that specifically binds to IL10Rb (an anti-IL10Rb sdAb) and a sdAb that specifically binds to IL28RA (an anti-IL28RA sdAb).

2. The IFNlambdaR1 binding molecule of claim 1, wherein the anti-IL10Rb sdAb is a V_{H}H antibody and/or the anti-IL28RA sdAb is a VHH antibody.

3. The IFNlambdaR1 binding molecule of claim 1, wherein the anti-IL10Rb sdAb and the anti-IL28RA sdAb are joined by a peptide linker.

4. The IFNlamdaR binding molecule of claim 3, wherein the peptide linker comprises between 1 and 50 amino acids.

5. The IFNlamdaR binding molecule of claim 4, wherein the peptide linker comprises a sequence of GGGS (SEQ ID NO: 13).

6. The IFNlamdaR1 binding molecule of claim 2, wherein the anti-IL10Rb sdAb comprises one or more CDRs in a row of Table 2 or Table 3, wherein each CDR independently comprises 0, 1, 2, or 3 amino acid changes relative to the sequence of Table 2 or Table 3.

7. The IFNlamdaR1 binding molecule of claim 2, wherein the anti-IL28RA sdAb comprises one or more CDRs in a row of Table 4 wherein each CDR independently comprises 0, 1, 2, or 3 amino acid changes relative to the sequence in a row of Table 4.

8. The IFNlamdaR1 binding molecule of claim 2, wherein the IFNlamdaR binding molecule comprises
an anti-IL10Rb sdAb comprising a CDR1, a CDR2, and a CDR3 in a row of Table 2 or Table 3 and
an anti-IL28RA sdAb a CDR1, a CDR2, and a CDR3 in a row of Table 4.

9. The IFNlamdaR1 binding molecule of claim 1, wherein the binding molecule comprises an anti-IL10Rb sdAb linked to the N-terminus of a linker and an anti-IL28RA sdAb linked to the C-terminus of the linker.

10. The IFNlamdaR1 binding molecule of claim 1, wherein the binding molecule comprises an anti-IL28RA sdAb linked to the N-terminus of a linker and an anti-IL10Rb sdAb linked to the C-terminus of the linker.

11. The IFNlamdaR1 binding molecule of claim 9, wherein the anti-IL10Rb sdAb comprises a sequence having at least 90% sequence identity to a sequence of Table 5 or Table 6.

12. The IFNlamdaR1 binding molecule of claim 9, wherein the anti-IL10Rb sdAb comprises a sequence of Table 5 or Table 6.

13. The IFNlamdaR1 binding molecule of claim 9, wherein the anti-IL28RA sdAb comprises a sequence having at least 90% sequence identity to a sequence of Table 7.

14. The IFNlamdaR1 binding molecule of claim 9, wherein the anti-IL28RA sdAb comprises a sequence of Table 7.

15. The IFNlamdaR1 binding molecule of claim 9, wherein each of the anti-IL10Rb sdAb comprises a sequence having at least 90% sequence identity to a sequence of Table 5 or Table 6 and the anti-IL28RA sdAb comprises a sequence having at least 90% sequence identity to a sequence of Table 7.

16. The IFNlamdaR1 binding molecule of claim 9, wherein each of the anti-IL10Rb sdAb comprises a sequence having at least 90% sequence identity to a sequence of Table 5 and the anti-IL28RA sdAb comprises a sequence having at least 90% sequence identity to a sequence of Table 7.

17. The IFNlamdaR1 binding molecule of claim 9, wherein each of the anti-IL10Rb sdAb comprises a sequence of Table 5 or Table 6 and the anti-IL28RA sdAb comprises a sequence of Table 7.

18. An isolated nucleic acid encoding the IFNlamdaR1 binding molecule of claim 1.

19. The isolated nucleic acid of claim 18, wherein the isolated nucleic acid comprises a sequence having at least 90% sequence identity to a sequence of Table 8 or Table 9 and the anti-IL28RA sdAb comprises a sequence having at least 90% sequence identity to a sequence of Table 10.

20. An expression vector comprising the nucleic acid of claim 18.

21. An isolated host cell comprising the vector of claim 20.

22. A pharmaceutical composition comprising the IFNlamdaR1 binding molecule of claim 1 and a pharmaceutically acceptable carrier.

23. A method of treating an autoimmune or inflammatory disease, disorder, or condition or a viral infection in a subject in need thereof, comprising administering to the subject a therapeutically effective amount of an IFNlamdaR1 binding molecule of claim 1.

24. (canceled)

25. (canceled)

26. (canceled)
